(19) United States
(12) Patent Application Publication
(10) Pub. No.: US 2007/0213283 A1
(43) Pub. Date: Sep. 13, 2007

(54) Macrolides substituted at the 4"-position

(76) Inventors: Sulejman Alihodzic, Zagreb (HR); Andrea Berdik, Zagreb (HR); John Michael Berge, Essex (GB); Richard Lewis Jarvest, Hertfordshire (GB); Stjepan Mutak, Zagreb (HR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/556,381

(22) PCT Filed: May 11, 2004
(86) PCT No.: PCT/EP04/05081

§ 371 Date: Dec. 13, 2006

(30) Foreign Application Priority Data
May 13, 2003 (GB) 0310984.0

(51) Int. Cl. C07H 17/08; A61K 31/7052

(52) U.S. Cl. 514/28; 536/7.4

(57) ABSTRACT

The present invention relates to 14- or 15-membered macrolides substituted at the 4″ position of formula (I) 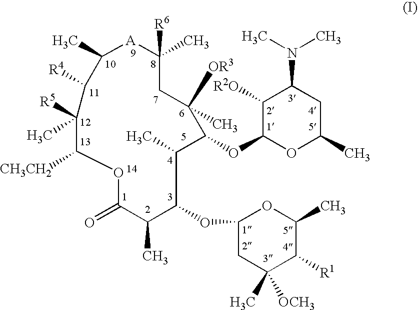 and pharmaceutically acceptable derivatives thereof, to processes for their preparation and their use in therapy or prophylaxis of systemic or topical microbial infections in a human or animal body.

The present invention relates to novel semi-synthetic macrolides having antimicrobial activity, in particular antibacterial activity. More particularly, the invention relates to 14- and 15-membered macrolides substituted at the 41 position, to processes for their preparation, to compositions containing them and to their use in medicine.

Macrolide antibacterial agents are known to be useful in the treatment or prevention of bacterial infections. However, the emergence of macrolide-resistant bacterial strains has resulted in the need to develop new macrolide compounds. For example, EP 0 895 999 describes derivatives modified at the 4′ position of the macrolide ring having antibacterial activity.

According to the present invention, we have now found novel 14- and 15-membered macrolides substituted at the 4″ position which also have antimicrobial activity.

Thus, the present invention provides compounds of general formula (I) 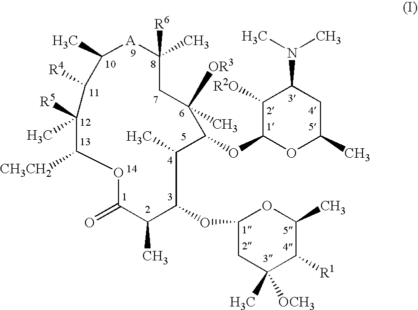
wherein
A is a bivalent radical selected from —C(O)—, —C(O)NH—, —NHC(O)—, —N(R⁷)—CH₂—, —CH₂—N(R⁷)—, —CH(NR⁸R⁹)— and —C(═NR¹⁰)—;
R¹ is —OC(O)(CH₂)_{d}XR¹¹;
R² is hydrogen or a hydroxyl protecting group;
R³ is hydrogen, C₁₋₄alkyl, or C₃₋₆alkenyl optionally substituted by 9 to 10 membered fused bicyclic heteroaryl;
   R⁴ is hydroxy, C₃₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R¹²,
   R⁵ is hydroxy, or
   R⁴ and R⁵ taken together with the intervening atoms form a cyclic group having the following structure: 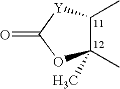
   wherein Y is a bivalent radical selected from —CH₂—, —CH(CN), —O—, —N(R¹⁻³ and —CH(SR¹³)—;
   R⁶ is hydrogen or fluorine;
   R⁷ is hydrogen or C₁₋₆alkyl;
   R⁸ and R⁹ are each independently hydrogen, C₁₋₆alkyl, —C(═NR¹⁰)NR¹⁴R¹⁵ or —C(O)R¹⁴, or
   R⁸ and R⁹ together form ═CH(CR¹⁴R¹⁵)_{f}aryl, ═CH(CR¹⁴R¹⁵)_{f}heterocycyl, ═CR¹⁴R¹⁵ or ═C(R¹⁴)C(O)OR¹⁴, wherein the alkyl, aryl and heterocyclyl groups are optionally substituted by up to three groups independently selected from R¹⁶;
   R¹⁰ is —OR¹⁷, C₁₋₆alkyl, —(CH₂)_{g}aryl, —(CH₂)_{g}heterocyclyl or —(CH₂)ₕO(CH₂)ᵢOR⁷, wherein each R¹⁰ group is optionally substituted by up to three groups independently selected from R¹⁶;
   R¹¹ is a heterocyclic group having the following structure: 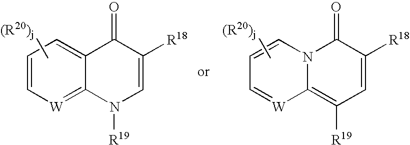
   R¹² is hydrogen or C₁₋₆alkyl;
   R¹³ is hydrogen or C₁₋₄alkyl optionally substituted by a group selected from optionally substituted phenyl, optionally substituted 5 or 6 membered heteroaryl and optionally substituted 9 to 10 membered fused bicyclic heteroaryl;
   R¹⁴ and R¹⁵ are each independently hydrogen or C₁₋₆alkyl;
   R¹⁶ is halogen, cyano, nitro, trifluoromethyl, azido, —C(O)R²¹, —C(O)OR²¹, —OC(O)R²¹, —OC(O)OR²¹, —NR²²C(O)R²³, —C(O)NR²²R²³, —NR²²R²³, hydroxy, C₁₋₆alkyl, —S(O)ₖC₁₋₆alkyl C₁₋₆alkoxy, —(CH₂)ₘaryl or —(CH₂)ₘheteroaryl, wherein the alkoxy group is optionally substituted by up to three groups independently selected from —NR¹⁴R¹⁵, halogen and —OR¹⁴; and the aryl and heteroaryl groups are optionally substituted by up to five groups independently selected from halogen, cyano, nitro, trifluoromethyl, azido, —C(O)R²⁴, —C(O)OR²⁴, —OC(O)OR²⁴, —NR²⁵C(O)R²⁶, —C(O)NR²⁵R²⁶, —NR²⁵R²⁶ hydroxy, C₁₋₆alkyl and C₁₋₆alkoxy;
   R¹⁷ is hydrogen, C₁₋₆alkyl, C₃₋₇cycloalkyl, C₃₋₆alkenyl or a 5 or 6 membered heterocyclic group, wherein the alkyl, cycloalkyl, alkenyl and heterocyclic groups are optionally substituted by up to three substituents independently selected from optionally substituted 5 or 6 membered heterocyclic group, optionally substituted 5 or 6 membered heteroaryl, —OR²⁷, —S(O)ₙR²⁷, —NR²⁷R²⁸, —CONR²⁷R²⁸, halogen and cyano;
   R¹⁸ is hydrogen, —C(O)OR²⁹, —C(O)NHR²⁹, —C(O)CH₂NO₂ or —C(O)CH₂SO₂R⁷;
   R¹⁹ is hydrogen, C₁₋₄alkyl optionally substituted by hydroxy or C₁₋₄alkoxy, C₃₋₇cycloalkyl, or optionally substituted phenyl or benzyl;
   R²⁰ is halogen, C₁₋₄alkyl, C₁₋₄thioalkyl, C₁₋₄alkoxy, —NH₂, —NH(C₁₋₄alkyl) or —N(C₁₋₄alkyl)₂;
   R²¹ is hydrogen, C₁₋₁₀alkyl, —(CH₂)ₚaryl or —(CH₂)ₚheteroaryl;
   R²² and R²³ are each independently hydrogen, —OR¹⁴, C₁₋₆alkyl, —(CH₂)_{q}aryl or —(CH₂)_{q}heterocyclyl;
   R²⁴ is hydrogen, C₁₋₁₀alkyl, —(CH₂)ᵣaryl or —(CH₂)ᵣheteroaryl;
   R²⁵ and R²⁶ are each independently hydrogen, —OR¹⁴, C₁₋₆alkyl, —(CH₂)ₛaryl or —(CH₂)ₛheterocyclyl;
   R²⁷ and R²⁸ are each independently hydrogen, C₁₋₄alkyl or C₁₋₄alkoxyC₁₋₄alkyl;
   R²⁹ is hydrogen,
   C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆-alkyl, —OC(O)OC₁₋₆alkyl, —C(O)NR³²R³³, —NR³²R³³ and phenyl optionally substituted by nitro or —C(O)OC₁₋₆alkyl,
   —(CH₂)_{w}C₃₋₇cycloalkyl,
   —(CH₂)_{w}heterocycyl,
   —(CH₂)_{w}heteroaryl,
   —(CH₂)_{w}aryl,
   C₃₋₆alkenyl, or
   C₃₋₆alkynyl;
      R³⁰ is hydrogen, C₁₋₄alkyl, C₃₋₇cycloalkyl, optionally substituted phenyl or benzyl, acetyl or benzoyl;
      R³¹ is hydrogen or R²⁰, or R³¹ and R¹⁹ are linked to form the bivalent radical —O(CH₂)₂— or —(CH₂)ₜ—;
      R³² and R³³ are each independently hydrogen or C₁₋₆alkyl optionally substituted by phenyl or —C(O)OC₁₋₆alkyl, or
      R³² and R³³, together with the nitrogen atom to which they are bound, form a 5 or 6 membered heterocyclic group optionally containing one additional heteroatom selected from oxygen, nitrogen and sulfur;
      X is —U(CH₂)ᵥB—;
      U is —N(R³⁰)— and B is —O— or —S(O)_{z}, or
      U is —O— and B is —N(R³⁰)— or —O—;
      W is —C(R³¹) or a nitrogen atom;
      d is 0 or an integer from 1 to 5;
      e is an integer from 2 to 4;
      f, g, h, m, p, q, r and s are each independently integers from 0 to 4;
      i is an integer from 1 to 6;
      j, k, n and z are each independently integers from 0 to 2;
      t is 2 or 3;
      v is an integer from 1 to 8;
      and pharmaceutically acceptable derivatives thereof.

According to another embodiment the present invention provides compounds of general formula (IA): 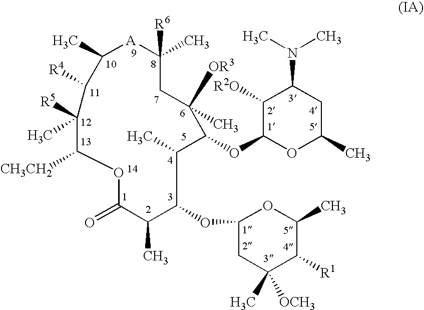
wherein
A is a bivalent radical selected from —C(O), —C(O)NH—, —NHC(O)—, —N(R⁷)—CH₂—, —CH₂—N(R⁷)—, —CH(NR⁸R⁹)— and —C(═NR¹⁰—;
R¹ is —OC(O)(CH₂)_{d}XR¹¹;
R² is hydrogen or a hydroxyl protecting group;
R³ is hydrogen, C₁₋₄alkyl, or C₃₋₆alkenyl optionally substituted by 9 to 10 membered fused bicyclic heteroaryl;
R⁴ is hydroxy, C₃₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R¹²,
R⁵ is hydroxy, or
R⁴ and R⁵ taken together with the intervening atoms form a cyclic group having the following structure: 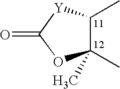
wherein Y is a bivalent radical selected from —CH₂, —CH(CN), —O—, —N(R¹³)— and —CH(SR¹³)—;
R⁶ is hydrogen or fluorine;
R⁷ is hydrogen or C₁₋₆alkyl:
R⁸ and R⁹ are each independently hydrogen, C₁₋₆alkyl, —C(═NR¹⁰)NR¹⁴R¹⁵ or —C(O)R¹⁴, or
R⁸ and R⁹ together form ═CH(CR¹⁴R¹⁵)aryl, ═CH(CR¹⁴R¹⁵)_{f}heterocyclyl, ═CR¹⁴R¹⁵ or ═C(R¹⁴)C(O)OR¹⁴, wherein the alkyl, aryl and heterocyclyl groups are optionally substituted by up to three groups independently selected from R¹⁶;
R¹⁰ is OR¹⁷, C₁₋₆alkyl, —(CH₂)_{g}aryl, —(CH₂)_{g}heterocyclyl or —(CH₂)ₕO(CH₂)ᵢOR⁷, wherein each R¹⁰ group is optionally substituted by up to three groups independently selected from R¹⁶;
R¹¹ is a heterocyclic group having the following structure: 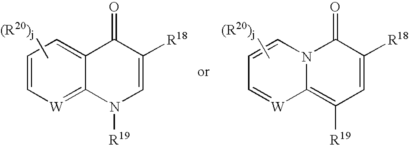
R¹² is hydrogen or C₁₋₆alkyl;
R¹³ is hydrogen or C₁₋₄alkyl substituted by a group selected from optionally substituted phenyl, optionally substituted 5 or 6 membered heteroaryl and optionally substituted 9 to 10 membered fused bicyclic heteroaryl;
R¹⁴ and R¹⁵ are each independently hydrogen or C₁₋₆alkyl;
R¹⁶ is halogen, cyano, nitro, trifluoromethyl, azido, —C(O)R²¹, —C(O)OR²¹, —OC(O)R²¹, —OC(O)OR²¹, —NR²²C(O)R²³, —C(O)NR²²R²³, —NR²²R²³, hydroxy, C₁₋₆alkyl, —S(O)ₖC₁₋₆alkyl, C₁₋₆alkoxy, —(CH₂)ₘaryl or —(CH₂)ₘheteroaryl, wherein the alkoxy group is optionally substituted by up to three groups independently selected from —NR¹⁴R¹⁵, halogen and CR¹⁴, and the aryl and heteroaryl groups are optionally substituted by up to five groups independently selected from halogen, cyano, nitro, trifluoromethyl, azido, —C(O)R²⁴, —C(O)OR²⁴, OC(O)OR²⁴, —NR²⁵C(O)R²⁶, —C(O)NR²⁵R²⁶, —NR²⁵R²⁶, hydroxy, C₁₋₆alkyl and C₁₋₆alkoxy;
R¹⁷ is hydrogen, C₁₋₆alkyl, C₃₋₇cycloalkyl, C₃₋₆alkenyl or a 5 or 6 membered heterocyclic group, wherein the alkyl, cycloalkyl, alkenyl and heterocyclic groups are optionally substituted by up to three substituents independently selected from optionally substituted 5 or 6 membered heterocyclic group, optionally substituted 5 or 6 membered heteroaryl, —OR²⁷, S(O)ₙR²⁷, —NR²⁷R²⁸, —CONR²⁷R²⁸ halogen and cyano;
R¹⁸ is hydrogen, —C(O)OR²⁹, —C(O)NHR²⁹ or —C(O)CH₂NO₂;
R¹⁹ is hydrogen, C₁₋₄alkyl optionally substituted by hydroxy or C₁₋₄alkoxy, C₃₋₇cycloalkyl, or optionally substituted phenyl or benzyl;
R²⁰ is halogen, C₁₋₄alkyl, C₁₋₄thioalkyl, C₁₋₄alkoxy, —NH₂, —NH(C₁₋₄alkyl) or —N(C₁₋₄alkyl)₂;
R²¹ is hydrogen, C₁₋₁₀alkyl, —(CH₂)ₚaryl or —(CH₂)ₚheteroaryl;
R²² and R²³ are each independently hydrogen, —OR¹⁴, C₁₋₆alkyl, —(CH₂)_{q}aryl or —(CH₂)_{q}heterocyclyl;
R²⁴ is hydrogen, C₁₋₁₀alkyl, —(CH₂)ᵣaryl or —(CH₂)ᵣheteroaryl;
R²⁵ and R²⁶ are each independently hydrogen, —OR¹⁴, C₁₋₄alkyl, —(CH₂)ₛaryl or —(CH₂)ₛheterocyclyl;
R²⁷ and R²⁸ are each independently hydrogen, C₁₋₄alkyl or C₁₋₄alkoxyC₁₋₄alkyl;
R²⁹ is hydrogen or C₁₋₆alkyl optionally substituted by up to three groups independently selected from halogen, C₁₋₄alkoxy, —OC(O)C₁₋₆alkyl and —OC(O)OC₁₋₆alkyl;
R³⁰ is hydrogen, C₁₋₄alkyl, C₃₋₇cycloalkyl, optionally substituted phenyl or benzyl, acetyl or benzoyl;
R³¹ is hydrogen or R²⁰, or R³¹ and R¹⁹ are linked to form the bivalent radical —O(CH₂)₂— or —(CH₂)ₜ—;
X is —U(CH₂)ᵥB—;
U is —N(R³⁰) and B is —O— or —S(O)_{z}, or
U is —O— and B is —N(R³⁰)— or —O—;
W is —C(R³¹)— or a nitrogen atom;
d is 0 or an integer from 1 to 5;
e is an integer from 2 to 4;
f, g, h, m, p, q, r and s are each independently integers from 0 to 4;
i is an integer from 1 to 6;
j, k, n and z are each independently integers from 0 to 2;
t is 2 or 3;
v is an integer from 2 to 8;
and pharmaceutically acceptable derivatives thereof.

The term “pharmaceutically acceptable” as used herein means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters, in particular salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula (I) may form solvates (e.g. hydrates) and the invention also includes all such solvates.

The term “prodrug” as used herein means a compound which is converted within the body, e.g. by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series, Edward B. Roche, ed., “Bioreversible Carriers in Drug Design”, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of structure (I) have more than one asymmetric carbon atom. In the general formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. A stereoisomeric mixture of the agent may also be prepared from a corresponding optically pure intermediate or by resolution, such as H.P.L.C., of the corresponding mixture using a suitable chiral support or by fractional crystallisation of the diastereoisomeric salts formed by reaction of the corresponding mixture with a suitable optically active acid or base, as appropriate.

The compounds of structure (I) may be in crystalline or amorphous form. Furthermore, some of the crystalline forms of the compounds of structure (I) may exist as polymorphs, which are included in the present invention.

Compounds wherein R² represents a hydroxyl protecting group are in general intermediates for the preparation of other compounds of formula (I).

When the group OR² is a protected hydroxyl group this is conveniently an ether or an acyloxy group. Examples of particularly suitable ether groups include those in which R² is a trialkylsilyl (i.e. trimethylsilyl). When the group OR² represents an acyloxy group, then examples of suitable groups R² include acetyl or benzoyl.

R⁶ is hydrogen or fluorine. However, it will be appreciated that when A is —C(O)NH— or —CH₂N(R⁷)—, R⁶ is hydrogen.

When R¹¹ is a heterocyclic group having the following structure: 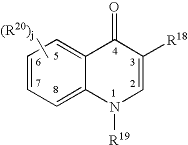
said heterocyclic is linked in the 6 or 7 position to the X group as above defined. When present, the R²⁰ group or groups may be attached at any position on the ring. In one embodiment, an R²⁰ group is attached at the 6 or 7 position.

When R¹¹ is a heterocyclic group having the following structure: 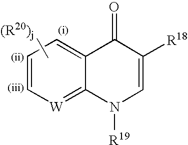
wherein W is —C(R³¹)— where R³¹ is R²⁰ or R³¹ and R¹⁹ are linked to form the bivalent radical —(CH₂)₂— or —(CH₂)ₜ—, said heterocyclic is linked in the (ii) or (iii) position to the X group as above defined.

When R¹¹ is a heterocyclic group having the following structure: 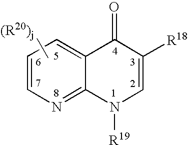
said heterocyclic is linked in the 6 or 7 position to the X group as defined above.

When R¹¹ is a heterocyclic group having the following structure: 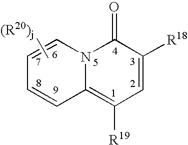
said heterocyclic is linked in the 7 or 8 position to the X group as above defined.

When R¹¹ is a heterocyclic group having the following structure: 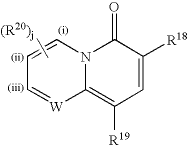
wherein W is —C(R³¹)— where R³¹ is R²⁰ or R³¹ and R¹⁹ are linked to form the bivalent radical —O(CH₂)₂ or —(CH₂)ₜ—, said heterocyclic is linked in the (ii) or (iii) position to the X group as above defined.

When R¹¹ is a heterocyclic group having the following structure: 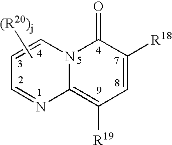
said heterocyclic is linked in the 2 or 3 position to the X group as above defined.

The term “alkyl” as used herein as a group or a part of a group refers to a straight or branched hydrocarbon chain containing the specified number of carbon atoms. For example, C₁₋₁₀alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄alkyl group is preferred, for example methyl, ethyl, n-propyl, isopropyl, n-butyl, isobutyl or t-butyl.

The term “C₃₋₇cycloalkyl” group as used herein refers to a non-aromatic monocyclic hydrocarbon ring of 3 to 7 carbon atoms such as, for example, cyclopropyl, cyclobutyl, cyclopentyl, cyclohexyl or cycloheptyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Similarly, the term “C₃₋₆alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-phenyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₃₋₆alkenyl” means a straight or branched alkynyl containing at least 3, and at most 6, carbon atoms containing at least one triple bond. Examples of “alkynyl” as used herein include, but are not limited to, propynyl, 1-butynyl, 2-butynyl, 1-pentynyl and 3-methyl-1-butynyl.

The term “aryl” as used herein refers to an aromatic carbocyclic moiety such as phenyl, biphenyl or naphthyl.

The term “heteroaryl” as used herein, unless otherwise defined, refers to an aromatic heterocycle of 5 to 10 members, having at least one heteroatom selected from nitrogen, oxygen and sulfur, and containing at least 1 carbon atom, including both mono and bicyclic ring systems. Examples of heteroaryl rings include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, thiadiazolyl, pyridyl, pyridazinyl, pyrazinyl, pyrimidinyl, triazinyl, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furyipyridine, oxazolopyridyl and benzothiophenyl.

The term “5 or 6 membered heteroaryl” as used herein as a group or a part of a group refers to a monocyclic 5 or 6 membered aromatic heterocycle containing at least one heteroatom independently selected from oxygen, nitrogen and sulfur. Examples include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, pyridyl, pyridazinyl, pyrazinyl, pyrimidinyl and triazinyl.

The term “9 to 10 membered fused bicyclic heteroaryl” as used herein as a group or a part of a group refers to quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl or benzothiophenyl.

The term “heterocycyl” as used herein, unless otherwise defined, refers to a monocyclic or bicyclic three- to ten-membered saturated or non-aromatic, unsaturated hydrocarbon ring containing at least one heteroatom selected from oxygen, nitrogen and sulfur. Preferably, the heterocyclyl ring has five or six ring atoms. Examples of heterocycyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “5 or 6 membered heterocyclic group” as used herein as a group or part of a group refers to a monocyclic 5 or 6 membered saturated hydrocarbon ring containing at least one heteroatom independently selected from oxygen, nitrogen and sulfur. Examples of such heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “halogen” refers to a fluorine, chlorine, bromine or iodine atom.

The terms “optionally substituted phenyl”, “optionally substituted phenyl or benzyl”, “optionally substituted 5 or 6 membered heteroaryl”, “optionally substituted 9 to 10 membered fused bicyclic heteroaryl” or “optionally substituted 5 or 6 membered heterocyclic group” as used herein refer to a group which is substituted by 1 to 3 groups selected from halogen, C₁₋₄alkyl, C₁₋₄alkoxy, hydroxy, nitro, cyano, amino, C₁₋₄alkylamino or diC₁₋₄alkylamino, phenyl and 5 or 6 membered heteroaryl.

In one embodiment, A is —C(O)—, —C(O)NH—, —NHC(O)—, —N(R⁷)—CH₂—, —CH₂—N(R⁷)— or —CH(NR⁸R⁹)—. In another embodiment, A is —C(O)—, —C(O)NH—, —NHC(O)—, —CH₂—N(R⁷y, —CH(NR⁸R⁹)— or —C(═NR¹⁰)—. In another embodiment, A is —C(O)—, —C(O)NH—, —NHC(O)—, —CH₂NR⁷— or —CH(NR⁸R⁹). In a further embodiment, A is —C(O)—, —N(R⁷)—CH₂ or —C(═NR¹⁰). Representative examples of A include —C(O)— and —N(R⁷)—CH₂—. A further representative example of A is —C(═NR¹⁰).

In one embodiment, R² is hydrogen or propionyl. A representative example of R² is hydrogen. A further representative example of R² is propionyl.

Representative examples of R³ include hydrogen and C₁₋₄alkyl, in particular hydrogen and methyl.

In one embodiment, R⁴ is hydroxy or C₁₋₆alkoxy, in particular hydroxy or methoxy. Preferably, R⁴ is hydroxy. In another embodiment, R⁵ is hydroxy. Alternatively, R⁴ and R⁵ taken together with the intervening atoms form a cyclic group having the following structure: 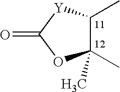
wherein Y is a bivalent radical selected from —O— and —N(R¹³)—.

A representative example of R⁶ is hydrogen.

A representative example of R⁷ is C₁₋₆alkyl, for example C₁₋₄alkyl, in particular methyl.

A representative example of R¹⁰ is —OR¹⁷.

Representative examples of R¹¹ include heterocyclic groups having the following structures: 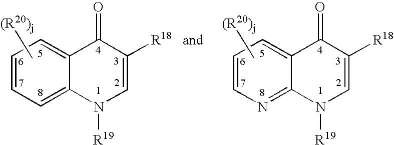
wherein the heterocyclic is linked in the 6 or 7 position to the X group as above defined, and heterocyclic groups having the following structure: 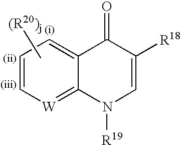
wherein W is —C(R³¹)— and R³¹ and R¹⁹ are linked to form the bivalent radical —(CH₂)ₜ—, and the heterocylic is linked in the (ii) or (iii) position to the X group as above defined.

In one embodiment, R¹³ is hydrogen or C₁₋₄alkyl substituted by a group selected from optionally substituted phenyl, optionally substituted 5 or 6 membered heteroaryl and optionally substituted 9 to 10 membered fused bicyclic heteroaryl. A representative example of R¹³ is hydrogen.

A representative example of R¹⁷ is C₁₋₄alkyl, in particular ethyl and isopropyl.

In one embodiment, R¹⁸ is hydrogen, —C(O)OR²⁹, C(O)NHR²⁹ or —C(O)CH₂NO₂. In a further embodiment, R¹⁸ is —C(O)OR²⁹, —C(O)NHR²⁹ or —C(O)CH₂NO₂. A representative example of R¹⁸ is —C(O)OR²⁹.

Representative examples of R¹⁹ include C₁₋₄alkyl, in particular ethyl, and C₃₋₇cycloalkyl, in particular cyclopropyl.

In one embodiment, R²⁰ is halogen or C₁₋₄alkoxy. A representative example of R²⁰ is halogen, in particular chlorine or fluorine. A further representative example of R²⁰ is C₁₋₄alkoxy, in particular methoxy.

In one embodiment, R²⁹ is hydrogen or C₁₋₆alkyl optionally substituted by up to three groups independently selected from halogen, C₁₋₄alkoxy, —OC(O)C₁₋₆alkyl and —OC(O)OC₁₋₆alkyl. In a further embodiment, R²⁹ is hydrogen; C₁₋₆alkyl optionally substituted by up to three groups, for example one group, independently selected from cyano, C₁₋₄alkoxy optionally substituted by phenyl or C₁₋₄alkoxy, —C(O)C₁₋₄alkyl, —C(O)OC₁₋₄alkyl, —OC(O)C₁₋₄alkyl, —OC(O)OC₁ 4alkyl, —C(O)NR³²R³³, —NR³²R³³ and phenyl optionally substituted by nitro or —C(O)OC₁₋₄alkyl; —(CH₂)_{w}C₃₋₇cycloalkyl; or C₃₋₆alkenyl. Representative examples of R²⁹ include hydrogen or C₁₋₆-alkyl optionally substituted by OC(O)C₁₋₆alkyl, in particular hydrogen or C₁₋₄alkyl optionally substituted by OC(O)C₁₋₄alkyl, such as hydrogen, methyl optionally substituted by —OC(O)ₜ— butyl, or i-propyl. In particular, R²⁹ is hydrogen.

A representative example of R³⁰ is hydrogen.

A representative example of R³¹ is hydrogen, or R³¹ and R¹⁹ are linked to form the bivalent radical —(CH₂)ₜ—.

In one embodiment, U is —O— and B is —N(R³⁰)— or —O—.

Representative examples of Y include the bivalent radicals —O— and —N(R¹³)—.

A representative example of d is 1 to 3, for example 2.

In one embodiment, v is an integer from 2 to 8. A representative example of v is 2 to 4, for example 2.

Representative examples of j include 0 and 1. A further representative example of j is 2.

A representative example of t is 3.

Representative examples of w include 1 and 2.

A representative example of z is 0.

It is to be understood that the present invention covers all combinations of particular and preferred groups described hereinabove. It is also to be understood that the present invention encompasses compounds of formula (I) in which a particular group or parameter, for example R⁷, R¹⁴, R¹⁵, R¹⁶, R²⁰, R²¹, R²², R²³, R²⁴, R²⁵, R²⁶, R²⁷, R²⁸, R³², R³³, k, m, n, p, q, r, s and z may occur more than once. In such compounds it will be appreciated that each group or parameter is independently selected from the values listed.

Particularly preferred compounds of the invention are:
4″-O-{3-[2-(3-carboxy-1-ethyl-4-oxo-1,4-dihydro-6-quinolinylsulfanyl)ethylamino]propionyl}-6-O-methyl-erythromycin A;
4″-O-{3-[2-(3 carboxy-1-ethyl-4-oxo-1,4-dihydro-6-quinolinylsulfanyl)ethylamino]propionyl}-6-O-methyl-11-desoxy-11-(R)-amino-erythromycin A11,12-carbamate;
4″O-{3-[2-(3-carboxy-1-ethyl-4-oxo-1,4-dihydro-6-quinolinysulfanyl)ethylamino]propionyl}-azithromycin11,12-carbonate;
4″-O-{3-[2-(6-carboxy-7-oxo-2,3-dihydro-1H,7H-pyrido[3,2,1-ij]quinolin-9-yloxy)ethylamino]propionyl}-6-O-methyl-erythromycin A;
4″-O-{3-[2-(3-carboxy-1-ethyl-4-oxo-1,4-dihydro-7-quinolinyloxy)ethylamino]propionyl}-6-O-methyl-erythromycin A;
4″-O-{3-[2-(3-carboxy-7-chloro-1-cyclopropyl-4-oxo-1,4-dihydro-6-quinolinylamino)ethoxy]propionyl}azithromycin;
4″-O-{3-[2-(3-carboxy-1-cyclopropyl-4-oxo-1,4-dihydro-6-quinolinylamino)ethoxy]propionyl}azithromycin;
4″-O-{3-[2-(3-carboxy-7-chloro-1-cyclopropyl-4-oxo-1,4-dihydro-6-quinolinylamino)ethoxy]propionyl}-11-O-methyl-azithromycin;
4″-O-{3-[2-(3 carboxy-7-chloro-1-cyclopropyl-4-oxo-1,4-dihydro-quinolin-6-yloxy)-ethoxy]-propionyl}-azithromycin;
4″-O-{3-[2-(3-carboxy-1-cyclopropyl-4-oxo-1,4-dihydro-quinolin-6-yloxy)ethoxy]-propionyl}-azithromycin;
   and pharmaceutically acceptable derivatives thereof.

Further particularly preferred compounds of the invention are:
4″-O-{3-[2-(3-carboxy-7-chloro-1-cyclopropyl-4-oxo-1,4-dihydro-6-quinolinylamino)ethoxy]propionyl}-azithromycin11,12-cyclic carbonate;
4″-O-{3-[2-(3-carboxy-1-cyclopropyl-4-oxo-1,4-dihydro-6-quinolinylamino)ethoxy]propionyl}-11-O-methyl-azithromycin;
4″-O-{3-[2-(3-carboxy-7-chloro-1-cyclopropyl-4-oxo-1,4-dihydro-quinolin-6-yloxy)ethoxy]-propionyl}-azithromycin11,12-carbonate;
4″-O-{3-[2-(3-carboxy-7-chloro-1-cyclopropyl-4-oxo-1,4-dihydro-6-quinolinylamino)ethoxy]propionyl}-6-O-methyl-11-desoxy-11-(R)-amino-erythromycin A11,12-carbamate;
4″-O-{3-[2-(3-carboxy-1-cyclopropyl-4-oxo-1,4-dihydro-quinolin-6-yloxy)ethoxy]-propionyl}-11-O-methyl-azithromycin;
4″-O-{3-[2-(3-carboxy-7-chloro-1-cyclopropyl-4-oxo-1,4-dihydro-quinolin-6-yloxy)ethoxy]propionyl}-6-O-methyl-erythromycin A;
4″-O-{3-[2-(3 carboxy-1-cyclopropyl-6-fluoro-8-methoxy-4-oxo-1,4-dihydro-quinolin-7-ylamino)ethoxy]propionyl}-azithromycin;
   and pharmaceutically acceptable derivatives thereof.

Compounds according to the invention also exhibit a broad spectrum of antimicrobial activity, in particular antibacterial activity, against a wide range of clinical pathogenic microorganisms. Using a standard microtiter broth serial dilution test, compounds of the invention have been found to exhibit useful levels of activity against a wide range of pathogenic microorganisims. In particular, the compounds of the invention may be active against strains of Staphylococcus aureus, Streptococcus pneumoniae, Moraxella catarrhalis, Streptococcus pyogenes, Haemophilus influenzae, Enterococcus faecalis, Chlamydia pneumoniae, Mycoplasma pneumoniae and Legionella pneumophila. The compounds of the invention may also be active against resistant strains, for example erythromycin resistant strains. In particular, the compounds of the invention may be active against erythromycin resistant strains of Streptococcus pneumoniae, Streptococcus pyogenes and Staphylococcus aureus.

The compounds of the invention may therefore be used for treating a variety of diseases caused by pathogenic microorganisms, in particular bacteria, in human beings and animals. It will be appreciated that reference to treatment includes acute treatment or prophylaxis as well as the alleviation of established symptoms.

Thus, according to another aspect of the present invention we provide a compound of formula (I) or a pharmaceutically acceptable derivative thereof for use in therapy.

According to a further aspect of the invention we provide a compound of formula (I) or a pharmaceutically acceptable derivative thereof for use in the therapy or prophylaxis of systemic or topical microbial infections in a human or animal subject.

According to a further aspect of the invention we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention we provide a method of treatment of the human or non-human animal body to combat microbial infections comprising administration to a body in need of such treatment of an effective amount of a compound of formula (I) or a pharmaceutically acceptable derivative thereof.

While it is possible that, for use in therapy, a compound of the invention may be administered as the raw chemical it is preferable to present the active ingredient as a pharmaceutical formulation eg when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising at least one compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another aspect, the invention provides a pharmaceutical composition comprising, as active ingredient, at least one compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof.

There is further provided by the present invention a process of preparing a pharmaceutical composition, which process comprises mixing at least one compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or carriers. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transmit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

It is to be understood that not all of the compounds need be administered by the same route. Likewise, if the composition comprises more than one active component, then those components may be administered by different routes.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, e.g. sterile, pyrogen-free water, before use.

The compounds of the invention can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories e.g. containing conventional suppository bases for use in human or veterinary medicine or as pessaries e.g. containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g. eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insuffator may be formulated to contain a powder mix of the compound and a suitable powder base such as lactose or starch.

For topical administration by inhalation the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may for example be used for topical administration with other active ingredients such as corticosteroids or antifungals as appropriate.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1 g of active ingredient. The duration of treatment will be dictated by the rate of response rather than by arbitrary numbers of days.

Compounds of general formula (I) and salts thereof may be prepared by the general methods outlined hereinafter, said methods constituting a further aspect of the invention. In the following description, the groups R¹ to R³³, A, B, X, Y, U, W, d, e, f, g, h, i, j, k, m, n, p, q, r, s, t, v, w and z have the meaning defined for the compounds of formula (I) unless otherwise stated.

The group X^{a}R^{11a} is XR¹¹ as defined for formula (I) or a group convertible to XR¹¹. Conversion of a group X^{a}R^{11a} to a XR¹¹ group typically arises if a protecting group is needed during the reactions described below. A comprehensive discussion of the ways in which such groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference. Examples of suitable amino protecting groups include acyl type protecting groups (e.g. formyl, trifluoroacetyl and acetyl), aromatic urethane type protecting groups (e.g. benzyloxycarbonyl (Cbz) and substituted Cbz, and 9-fluorenylmethoxycarbonyl (Fmoc)), aliphatic urethane protecting groups (e.g. t-butyloxycarbonyl (Boc), isopropyloxycarbonyl and cyclohexyloxycarbonyl) and alkyl type protecting groups (e.g. benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

Compounds of formula (I) wherein d is an integer from 1 to 5, may be prepared by reaction of a 4″ hydroxy compound of formula (II) wherein R² is a hydroxy protecting group with a suitable activated and protected derivative of the carboxylic acid (III), followed where necessary by subsequent removal of the hydroxyl protecting group R² and conversion of the X^{a}R^{11a} group to XR¹¹.

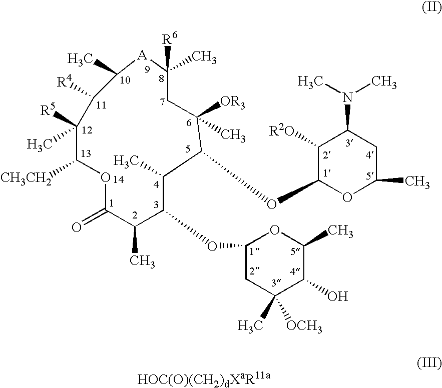

Suitable activated derivatives of the carboxyl group include the corresponding acyl halide, mixed anhydride or activated ester such as a thioester. The reaction is preferably carried out in a suitable aprotic solvent such as a halohydrocarbon (e.g. dichloromethane) or N,N-dimethylformamide optionally in the presence of a tertiary organic base such as dimethylaminopyridine or triethylamine or in the presence of inorganic base (eg sodium hydroxide) and at a temperature within the range of 0° to 120° C. The compounds of formula (II) and (III) may also be reacted in the presence of a carbodiimide such as dicyclohexylcarbodiimide (DCC).

Compounds of formula (I) wherein d is 0 and U is a group selected from —N(R³⁰)— and —O—, may be prepared by reaction of compounds of formula (II), in which the 4″ hydroxy is suitably activated, with a compound of formula X^{a}R^{11a} (IV) followed where necessary by subsequent removal of the hydroxyl protecting group R² and conversion of the X^{a}R^{11a} group to XR¹¹. Suitable activated derivatives of the 4″ hydroxy group include for example carbonyl imidazolide. The reaction is preferably carried out in a suitable aprotic solvent such as a halohydrocarbon (e.g. dichloromethane) or N,N-dimethylformamide optionally in the presence of a tertiary base such as 1,8-diazabicyclo[5.4.0]undec-7-ene (DBU), dimethylaminopyridine or triethylamine and at a temperature within the range of 0° to 120° C.

In a further embodiment of the invention, compounds of formula (I) wherein d is an integer from 1 to 5 and U is —N(R³⁰)—, may be prepared by reaction of compounds of formula (V), 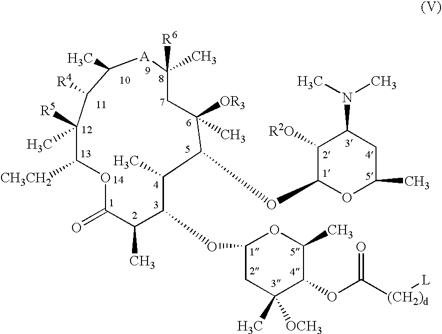
wherein d is an integer from 1 to 5 and L is a suitable leaving group, with X^{a}R^{11a} (IV) in which U is —N(R³⁰)—. The reaction is preferably carried out in a solvent such as a halohydrocarbon (e.g. dichloromethane), an ether (e.g. tetrahydrofuran or dimethoxyethane), acetonitrile or ethyl acetate and the like, dimethylsulfoxide, N,N-dimethylformamide or 1-methyl-pyrrolidone and in the presence of a base, followed, if desired, by removal of the hydroxyl protecting group R² and conversion of the X^{a}R^{11a} group to XR¹¹. Examples of the bases which may be used include organic bases such as diisopropylethylamine, triethylamine and 1,8-diazabicyclo[5.4.0]undec-7-ene, and inorganic bases such as potassium hydroxide, cesium hydroxide, tetraalkylammonium hydroxide, sodium hydride, potassium hydride and the like. Suitable leaving groups for this reaction include halide (e.g. chloride, bromide or iodide) or a sulfonyloxy group (e.g. tosyloxy or methanesulfonyloxy).

Compounds of formula (V) may be prepared by reaction of a compound of formula (II), wherein R² is a hydroxyl protecting group, with a suitable activated derivative of the carboxylic acid HOC(O)(CH₂)_{d}L (VI), wherein L is a suitable leaving group as above defined. Suitable activated derivatives of the carboxyl group are those defined above for carboxylic acid (III). The reaction is carried out using the conditions described above for the reaction of a compound of formula (II) with carboxylic acid (III).

In a preferred embodiment of the invention, compounds of formula (I) wherein d is 2 and U is —N(R³⁰)—, may be prepared by Michael reaction of a compound of formula (VII) wherein R² is optionally a hydroxy protecting group 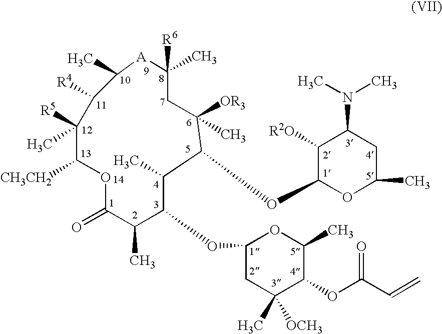
with a compound of formula X^{a}R^{11a} (IV). The reaction is suitably carried out in a solvent such as dimethylsulfoxide, N,N-dimethylformamide, 1-methyl-pyrrolidone, a halohydrocarbon (e.g. dichloromethane), an ether (e.g. tetrahydrofuran or dimethoxyethane), acetonitrile or alcohol (e.g. methanol or isopropanol) and the like, and in the presence of a base, followed, if desired, by removal of hydroxyl protecting group R² and conversion of the X^{a}R^{11a} group to XR¹¹.

Compounds of formula (I) may be converted into other compounds of formula (I). Thus compounds of formula (I) wherein B is —S(O)_{z}— and z is 1 or 2 may be prepared by oxidation of the corresponding compound of formula (I) wherein z is 0. The oxidation is preferably carried out using a peracid, e.g. peroxybenzoic acid, followed by treatment with a phosphine, such as triphenylphosphine. The reaction is suitably carried out in an organic solvent such as methylene chloride. Compounds of formula (I) wherein U or 8 is —N(R³⁰)—and R³⁰ is C₁₋₄alkyl can be prepared from compounds wherein R³⁰ is hydrogen by reductive alkylation.

Compounds of formula (II) wherein A is —C(O)NH— or —NHC(O)—, R⁴ or R⁵ are hydroxy, R³ is hydrogen and R⁶ is hydrogen are known compounds or they may be prepared by analogous methods to those known in the art. Thus they can be prepared according to the procedures described in EP 507595 and EP 503932.

Compounds of formula (II), wherein A is —C(O)NH— or —NHC(O)—, R⁴ or R⁵ are hydroxy and R³ is C₁₋₄alkyl or C₃₋₆alkenyl optionally substituted by 9 to 10 membered fused bicyclic heteroaryl and R⁶ is hydrogen are known compounds or they may be prepared by analogous methods to those known in the art. Thus they can be prepared according to the procedures described in WO 9951616 and WO 0063223.

Compounds of formula (II), wherein A is —C(O)NH—, R⁴ and R⁵ taken together with the intervening atoms form a cyclic group having the following structure:

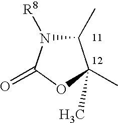

R³ is C₁₋₄alkyl, or C₃₋₆alkenyl optionally substituted by 9 to 10 membered fused bicyclic heteroaryl and R⁶ is hydrogen are known compounds or they may be prepared by analogous methods to those known in the art. Thus they can be prepared according to the procedures described in U.S. Pat. No. 6,262,030.

Compounds of formula (II), wherein A is —C(O)—, —C(O)NH—, —NHC(O)—, —N(R⁷)—CH₂—, —CH₂—N(R⁷)— or —CH(NR⁸R⁹)—, R⁴ or R⁵ are hydroxy or R⁴ and R⁵ taken together with the intervening atoms form a cyclic group having the following structure: 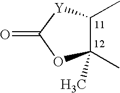
wherein Y is a bivalent radical selected from —O— and —N(R¹³)—, and R³ is C₁₋₄alkyl, or C₃₋₆alkenyl optionally substituted by 9 to 10 membered fused bicyclic heteroaryl are known compounds or they may be prepared by analogous methods to those known in the art. Thus they can be prepared according to the procedures described in EP 307177, EP 248279, WO 0078773, WO 9742204.

Compounds of formula (II), wherein A is —C(O)NH—, —NHC(O)—, —N(CH₃)—CH₂— or —CH₂—N(CH₃)—, R⁴ or R⁵ are hydroxy or R⁴ and R⁵ taken together with the intervening atoms form a cyclic group having the following structure: 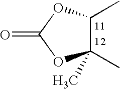
and R⁶ is hydrogen are known compounds or they may be prepared by analogous methods to those known in the art. Thus they can be prepared according to the procedures described in EP 508699 and J. Chem. Res. Synop (1988 pages 152-153), U.S. Pat. No. 6,262,030.

Compounds of formula (II), wherein A is —C(═NR¹⁰)—, R⁴ or R⁵ are hydroxy or R⁴ and R⁵ taken together with the intervening atoms form a cyclic group having the following structure: 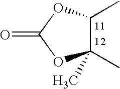
and R⁶ is hydrogen, are known compounds or they may be prepared by analogous methods to those known in the art. Thus they can be prepared according to the procedures described in EP 284203.

Compounds of formula (II), wherein A is —C(O)—, R⁴ and R⁵ taken together with the intervening atoms form a cyclic group having the following structure:

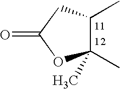

R⁶ is hydrogen and R³ is C₁₋₄ alkyl may be prepared by decarboxylation of a compound of formula (VIII), wherein R³⁴ is hydroxy protecting group followed, if required, by removal of the protecting group R² or R³⁴.

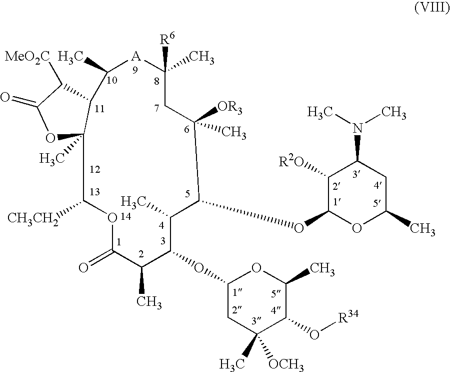

The decarboxylation may be carried out in the presence of a lithium salt such as lithium chloride, preferably in an organic solvent such as dimethylsulfoxide.

Compounds of formula (II), wherein A is —C(O)—, R⁴ and R⁵ taken together with the intervening atoms form a cyclic group having the following structure: 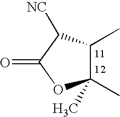
and R³ is C₁₋₄ alkyl may be prepared according to the procedures described in WO 02/50091 and WO 02/50092.

Compounds of formula (III) wherein X is —U(CH₂)ᵥN(R³⁰)—, in which U is —O—, may be prepared by reaction of X^{a}R^{11a} (IV), wherein X has the meaning defined above with R³⁵OC(O)(CH₂)_{d}L (IX) wherein R³⁵ is carboxyl protecting group and L is a suitable leaving group, followed by removal of R³⁵. Suitable R³⁵ carboxyl protecting group include t-butyl, allyl or benzyl.

Compounds of formula (IV) wherein X is —U(CH₂)ᵥB— in which B is —N(R³⁰)—, —O— or —S—, may be prepared by reaction of a compound of formula R^{11a}L (X), wherein L is a suitable leaving group such as chlorine, fluorine or bromine, with a compound of formula —U(CH₂)ᵥB (XI) in which B is —N(R³⁰)—, —O— or —S—, or with piperazine or with 1H-pyrrolo[3,4-b]pyridine, octahydro.

In order that the invention may be more fully understood the following examples are given by way of illustration only.

The following abbreviations are used in the text: BOC for t-butoxycarbonyl, BTEAC for benzyltriethylammonium chloride, DBU for 1,8-diazabicyclo[5.4.0]undec-7-ene, DCM for dichloromethane, DMAP for 4-dimethylaminopyridine, DMF for N,N-dimethylformamide, DMSO for dimethyl sulfoxide, EDC.HCl for 1-(3-dimethylaminopropyl)-3-ethylcarbodiimide hydrochloride, EtOAc for ethyl acetate, EtOH for ethanol, Fmoc for 9-fluorenylmethoxycarbonyl, HOBt for 1-hydroxybenzotriazole hydrate, i-PrOH for isopropanol, KO^{t}Bu for potassium tert-butoxide, MEM-chloride for methoxyethoxymethyl chloride, MeOH for methanol, TEA for triethylamine and THF for tetrahydrofuran.

### EXAMPLES

2′-O-Acetyl-6-O-methyl-erythromycin A (2′-O-acetyl-clarithromycin) may be prepared by the procedure described by W. R. Baker et al. in J. Org. Chem. 1988, 53, 2340, 2′-O-acetyl-azithromycin and 2′-O-acetyl-azithromycin-11,12-carbonate may be prepared by the procedures described by S. Djokic et al. in J. Chem. Res. (S) 1988, 152, 11-O-methyl-azithromycin may be prepared by the procedure described by G. Kobrehel et al. in J. Antibiotics, 45, 1992, 527-532 and 9(E)-O-(2-propyl)oximino erythromycin A may be prepared by the procedure described in EP 1 167 375.

### Intermediate 1: 7-(2-Amino-ethylsulfanyl)-1-ethyl-4-oxo-1,4-dihydro-quinoline-3-carboxylic acid trifluoroacetate salt

### a) Benzyl-2-(4-bromo-2-fluorobenzoyl)acetate

To mono benzyl malonate (9.7 g, 50 mmol) in THF (50 mL) was added magnesium ethoxide (2.85 g, 25 mmol). The mixture was sonicated until a uniform yellow suspension was formed. The solvent was removed by evaporation under reduced pressure and to the residue added a solution of 1-(4-bromo-2-fluorobenzoyl)imidazole prepared by treating 4-bromo-2-fluorobenzoic acid (10.95 g, 50 mmol) in THF (50 mL) with carbonyl diimidazole (8.1 g, 50 mmol) at 20° C. for 1 h and 50° C. for 1 h. After stirring at 20° C. for 20 h, the brown mixture was diluted with ethyl acetate and washed with water, 2M HCl and brine. The organic layer was dried and the solvent was removed under reduced pressure. To the residue was added dichloromethane/petrol (100 mL, 1:1), the mixture filtered, and the soluble material purified by chromatography (silica gel) eluting with 0-10% ethyl acetate/hexane to give the title product (9.85 g, 56%); ESMS m/z 351, 353 [M+H]⁺ (100%).

### b) Benzyl 7-bromo-1 ethyl-4-oxo-1,4-dihydro-quinoline-3-carboxylate

To a solution of Intermediate 1a (9.82 g, 28 mmol) in THF (50 mL) was added dimethyl formamide dimethyl acetal (3.9 mL, 29 mmol). After 16 h at 20° C. the solvent was removed by evaporation under reduced pressure, the residue was taken up in THF (20 mL), and cooled in an ice bath. Ethylamine in THF (2M, 14 mL) was added and the mixture stirred at 0° C. After 45 min the mixture was evaporated to dryness and the residue taken up in DMF (40 mL). Potassium carbonate (5.28 g, 38 mmol) was added and the mixture stirred and heated to 100° C. under argon. After 45 min the mixture was cooled and diluted with ice water to precipitate a yellow solid. The solid was dried and refluxed with ethyl acetate (100 mL) then the mixture filtered. This was repeated with further portions of ethyl acetate (100, 50 mL) and the combined filtrates diluted with hexane to give the title product. Further material was obtained by chromatography of the mother liquors and the ethyl acetate insoluble material on silica gel eluting with 0-20% In dichloromethane gave a total of the title product (2.65 g, 24%); ¹H NMR δ (CDCl₃/MeOD) 1.54 (3H, t), 4.35 (2H, q), 5.36 (2H, s), 7.3-7.5 (5H, m), 7.63 (1H, dd), 7.9 (1H, d), 8.33 (1H, d), 8.70 (1H, s).

### c) Benzyl 7-(2-tert-Butoxycarbonylaminoethylsulfanyl)-1-ethyl-4-oxo-1,4-dihydro-quinoline-3-carboxylate

A solution of Intermediate 1b (0.386 g, 1 mmol) and N-Boc-cysteinamine (0.354 g, 2 mmol) in DMSO (3 mL) was treated with potassium carbonate (0.278 g, 2 mmol) and stirred at 60° C. under argon for 3 h. The cooled mixture was diluted with ethyl acetate and washed with water. The crude product was purified by chromatography on silica gel eluting with 0-20% ethyl acetate in dichloromethane. The product containing fractions were precipitated from dichloromethane solution with hexane to give the title product (0.346 g, 72%); ¹H NMR δ (CDCl₃) 1.45 (9H, s), 1.52 (3H, t), 3.18 (2H, t), 3.39 (2H, m), 4.35 (2H, q), 4.95 (1H, bt), 5.4 (2H, s), 7.2-7.4 (4H, m), 7.52 (3H, m), 8.42 (1H, d), 8.47 (1H, s).

### d) 7-(2-Amino-ethylsulfanyl)-1-ethyl-4-oxo-1,4-dihydro-quinoline-3-carboxylic acid trifluoroacetate salt

Intermediate 1c (0.2 g, 0.415 mmol) was dissolved in THF, and aqueous sodium hydroxide solution (2M, 0.25 mL) added. The reaction was heated to 65° C. under argon. After 3 h further sodium hydroxide solution (2M, 0.25 mL) was added, and after a further 30 min methanol (1 drop) was added. After a total reaction time of 4.5 h the reaction was cooled to 20° C. The organic solvents were removed and a small piece of solid carbon dioxide added. The aqueous mixture was evaporated to dryness under reduced pressure and triturated with ethanol. The insoluble material (0.12 g) was treated with anisole (0.5 mL) and trifluoroacetic acid (2 mL) for 1 h. Toluene (10 mL) was added and the solution evaporated to low volume. Diethyl ether (10 mL) was added to give a precipitate of the title compound (0.12 g) which was used without further purification; ESMS m/z 293 [M+H]⁺ (100%).

### Intermediate 2: 6-(2-Amino-ethylsulfanyl)-1-ethyl-4-oxo-1,4-dihydro-[1,8]naphthyridine-3-carboxylic acid trifluoroacetate salt

### a) 1-Ethyl-6-fluoro-4-oxo-1,4-dihydro-[1,8]naphthyridine-3-carboxylic acid ethyl ester

7-Chloro-1-ethyl-6-fluoro-4-oxo-1,4-dihydro-[1,8]naphthyridine-3-carboxylic acid ethyl ester (0.245 g, 0.82 mmol) and triethylamine (0.228 mL, 1.6 mmol) in ethanol (15 mL) was hydrogenated at 1 atm and 20° C. over 5% palladium on carbon (200 mg) for 16 h. After filtration and removal of the solvent by evaporation under reduced pressure the residue was purified by chromatography on silica gel, eluting with 25-100% ethyl acetate in hexane, to give the title compound (0.16 g. 75%); ESMS m/z 265 [M+H]⁺ (100%).

### b) 6-(2-tert-Butoxycarbonylaminoethylsulfanyl)-1-ethyl-4-oxo-1,4-dihydro-[1,8]naphthyridine-3-carboxylic acid ethyl ester

A solution of Intermediate 2a (0.158 g, 0.6 mmol) in DMSO (5 mL) was stirred with N-Boc-cysteinamine (0.106 g, 0.6 mmol) and potassium carbonate (0.166 g, 1.2 mmol) at 50° C. under argon. A further portion of N-Boc-cysteinamine was added after 17 h (0.027 g, 0.15 mmol) and again after 24 h (0.05 g, 0.30 mmol). After 40 h reaction the mixture was cooled and diluted with water. Extraction with ethyl acetate gave a crude product which was purified by chromatography on silica gel, eluting with 20-100% ethyl acetate in hexane, to give the title compound (0.234 g, 93%); ESMS m/z 422 [M+H]⁺ (85%), 366 (100%).

### c) 6-(2-tert-Butoxycarbonylaminoethylsulfanyl)-1-ethyl-4-oxo-1,4-dihydro-[1,8]naphthyridine-3-carboxylic acid

Intermediate 2b (0.234 g, 0.56 mmol) in 1,4-dioxane (10 mL) was stirred with NaOH (2M, 0.28 mL, 0.56 mmol) at 20″C for 16 h, then 80° C. for 28 h. The reaction mixture was cooled, citric acid (5%) added and the solvent removed in vacuo to give a residue which was triturated with ethyl acetate and water. The resulting white material was removed by filtration and dried under vacuum to give the title compound as a cream solid (0.134 g, 61%); ESMS m/z 392 (M−H⁻, 100%).

### d) 6-(2-Amino-ethylsulfanyl)-1-ethyl-4-oxo-1,4-dihydro-[1,8]naphthyridine-3-carboxylic acid, trifluoroacetate salt

A suspension of Intermediate 2c (0.134 g, 0.34 mmol) in dichloromethane (10 mL) was treated with trifluoroacetic acid (5 mL). After 25 min the solvent was removed in vacuo. To the residue methanol was added and the resulting precipitate was removed by filtration and dried under vacuum to give title compound as a white solid (0.060 g, 43%); ESMS m/z 294 [M+H]⁺ (100%).

### Intermediate 3: 9-(2-Amino-ethoxy)-1-oxo-6,7-dihydro-1H,5H-pyrido[3,2,1-ij]quinoline-2-carboxylic acid hydrochloride

### a) 9-(2-Dibenzylamino-ethoxy)-1-oxo-6,7-dihydro-1H,5H-pyrido[3,2,1-ij]quinoline-2-carboxylic acid 2-dibenzylamino-ethyl ester

9-Hydroxy-1-oxo-6,7-dihydro-1H,5H-pyrido[3,2,1-ij]quinoline-2-carboxylic acid (GB1417129) (0.905 g, 3.69 mmol) was suspended in dry DMF (50 mL). To this was added potassium carbonate (3.06 g, 22 mmol) and dibenzyl-(2-chloroethyl)amine hydrochloride (2.37 g, 8 mmol). The mixture was heated at 60° C. for 16 h, then more potassium carbonate (0.55 g) and dibenzyl-(2-chloroethyl)amine hydrochloride (1.18 g, 4 mmol) were added. After a further 25 h at 75° C. the mixture was evaporated. The residue was diluted with water and extracted with ethyl acetate (×3). The combined organic extracts were washed with brine, dried and evaporated under reduced pressure. The crude product (4.0 g) was purified by chromatography on silica gel (100 g), eluting with 0-4% methanol in dichloromethane, to give the title compound (2.25 g, 89%); ESMS m/z 692 [M+H]⁺ (100%).

### b) 9-(2-Dibenzylamino-ethoxy)-1-oxo-6,7-dihydro-1H,5H-pyrido[3,2,1-ij]quinoline-2-carboxylic acid sodium salt

Intermediate 3a (2.22 g, 3.21 mmol) was dissolved in methanol (30 mL) and 1,4-dioxane (20 mL), and treated with aqueous sodium hydroxide (0.4N, 8.03 mL, 3.21 mmol). The mixture was stirred for 88 h at 20° C. Solid carbon dioxide was then added and the mixture evaporated to dryness under reduced pressure. The residue was triturated with diethyl ether to give the title compound as a white powder (1.6 g, 100%); ESMS m/z 469 [M+H]⁺ for free acid (100%).

### c) 9-(2-Amino-ethoxy)-1-oxo-6,7-dihydro-1H,5H-pyrido[3,2,1-ij]quinoline-2-carboxylic acid hydrochloride

Intermediate 3b (0.8 g, 1.63 mmol) was dissolved in 1,4-dioxane (100 mL), water (15 mL) and hydrochloric acid (2N, 1.6 mL, 3.2 mmol). This solution was hydrogenated over 20% palladium(II) hydroxide on carbon (0.4 g) at 50 psi for 42 h. The mixture was diluted with water and filtered through kieselguhr, washing well with water. The filtrate was then evaporated to dryness under reduced pressure to give the title compound as an off-white solid (0.54 g, 87%) (containing one equivalent of sodium chloride); ESMS m/z 289 [M+H]⁺ for free acid (100%).

### Intermediate 4: 6-(2-Amino-ethoxy)-1-ethyl-4-oxo-1,4-dihydro-quinoline-3-carboxylic acid hydrochloride

### a) 6-(2-Dibenzylamino-ethoxy)-1-ethyl-4-oxo-1,4-dihydro-quinoline-3-carboxylic acid 2-dibenzylamino-ethyl ester

1-Ethyl-6-hydroxy-4-oxo-1,4-dihydro-quinoline-3-carboxylic acid (GB 1433774) (1.4 g, 6 mmol) was dissolved in dry DMF (80 mL). To this was added potassium carbonate (5 g, 36 mmol) and dibenzyl-(2-chloroethyl)amine hydrochloride (4.37 g, 14.8 mmol). The mixture was heated at 65° C. with stirring for 72 h, then allowed to cool overnight. The mixture was evaporated to a small volume, diluted with water and extracted with ethyl acetate (×2). The combined organic extracts were washed with brine, dried and evaporated under reduced pressure to give a dark viscous oil (4.9 g). This residue was purified by chromatography on silica gel (100 g), eluting with 0.2-3.8% methanol in dichloromethane, to give the title compound as a brown solid (2.46 g, 60%); ESMS m/z 680 [M+H]⁺ (100%).

### b) 6-(2-Dibenzylamino-ethoxy)-1,4-ethyl-4-oxo-1,4-dihydro-quinoline-3-carboxylic acid sodium salt

Intermediate 4a (2.44 g, 3.59 mmol) was dissolved in methanol (25 mL) and 1,4-dioxane (25 mL), then aqueous sodium hydroxide (0.4N, 8.75 mL, 3.5 mmol) was added. Stirred for 40 h then a little more sodium hydroxide was added and stirring continued for a further 72 h. Excess solid carbon dioxide was then added and the mixture evaporated to dryness under reduced pressure. Trituration with diethyl ether gave the title compound as a pale brown powder (1.382 g, 84%); ESMS m/z 457 [M+H]+for the free acid (100%).

### c) 6-(2-Amino-ethoxy)-1 ethyl-4-oxo-1,4-dihydro-quinoline-3-carboxylic acid

Intermediate 4b (1.38 g, 2.89 mmol) was dissolved in 1,4-dioxane (80 mL), water (40 mL) and hydrochloric acid (2N, 2.9 mL, 5.8 mmol). This solution was hydrogenated over 20% palladium(II) hydroxide on carbon (0.6 g) at 50 psi for 18 h. The mixture was filtered through kieselguhr, washing well with water. The filtrate was then evaporated to dryness under reduced pressure to give the title compound as a pale yellow solid (1 g, 94%) (containing one equivalent of sodium chloride); ESMS m/z 277 [M+H]⁺ for free acid (100%).

### Intermediate 5: 7-(2-Amino-ethoxy)-1-ethyl-4-oxo-1,4-dihydro-quinoline-3-carboxylic acid sodium salt

### a) 7-Benzyloxy-1-ethyl-4-oxo-1,4-dihydroquinoline-3-carboxylic acid ethyl ester

A mixture of 7-benzyloxy-4-oxo-1,4-dihydro-quinoline-3-carboxylic acid ethyl ester (0.97 g, 3 mmol) and potassium carbonate (0.56 g, 4 mmol) in DMF was stirred for 1 h at 50° C. under argon followed by addition of iodoethane (0.9 g, 12 mmol). After stirring for a further 14 h the mixture was cooled and the DMF evaporated. The residue was treated with water and cooled in ice. The resultant crystalline product was filtered and dried under vacuum overnight to yield the title compound as a white powder; ¹H NMR δ (CDCl₃) 1.42 (3H, t, J=7.2 Hz), 1.45 (3H, t, J=7.2 Hz), 4.14 (2H, q, J=7.2 Hz), 4.39 (2H, q, J=7.1 Hz), 5.20 (2H, s), 6.86 (1H, d, J=2.2 Hz), 7.11 (1H, dd, J=9.0 & 2.2 Hz), 7.3-7.5 (5H, m), 8.42(1H, s), 8.47 (1H, d, J=9.0 Hz).

### b) 1-Ethyl-7-hydroxy-4-oxo-1,4-dihydro-quinoline-3-carboxylic acid ethyl ester

A solution of Intermediate 5a (1.0 g, 2.8 mmol) in methanol (10 mL) was hydrogenated in the presence of 10% palladium on charcoal (50 mg) at 1 atmosphere and room temperature. After 14 h another 50 mg of catalyst was added. After a further 24 h the mixture was filtered and the methanol evaporated to yield the title compound as a pale yellow solid; ¹H NMR δ [(CD₃)₂SO] 1.28 (3H, t, J=7.1 Hz), 1.36 (3H, t, J=7.1 Hz), 4.20 (2H, q, J=7.1 Hz), 4.28 (2H, q, J=7.1 Hz), 6.92 (1H, dd, J=8.8 & 2.1 Hz), 6.97 (1H, d, J=2.1 Hz), 8.08 (1H, d, J=8.8 Hz), 8.57 (1H, s); 10.52 (1H, br. s).

### c) 7-(2-Dibenzylamino-ethoxy)-1-ethyl-4-oxo-1,4-dihydro-quinoline-3-carboxylic acid ethyl ester

Intermediate 5b (0.371 g, 1.42 mmol) was dissolved in dry DMF (10 mL) and to this was added potassium carbonate (0.588 g, 4.26 mmol) and dibenzyl-(2-chloroethyl)amine hydrochloride (0.462 g, 1.56 mmol). The mixture was heated at 70° C. for 5 h, evaporated to a small volume, diluted with water and extracted with ethyl acetate (×2). The combined organic extracts were washed with brine, dried and evaporated under reduced pressure to give a yellow oil (0.76 g). This residue was purified by chromatography on silica gel (40 g), eluting with 0-4% methanol in dichloromethane, to give the title compound as a cream solid (0.709 g, 100%); ESMS m/z 485 [M+H]⁺ (100%).

### d) 7-(2-Amino-ethoxy)-1-ethyl-4-oxo-1,4-dihydro-quinoline-3-carboxylic acid ethyl ester

A solution of Intermediate 5c (0.7 g, 1.42 mmol) in ethanol (70 mL) was hydrogenated over 20% palladium(II) hydroxide on carbon (0.26 g) at 50 psi for 31 h. More catalyst (0.2 g) was then added and hydrogenation continued for a further 22 h. The mixture was then filtered through kieselguhr, washing well with ethanol, and the filtrate evaporated to dryness under reduced pressure. The residue was purified by chromatography on silica gel (20 g), eluting with 0-8% methanolic ammonia (2M) in dichloromethane, to give the title compound as an off-white solid (0.239 g, 55%); ESMS m/z 305 [M+H]⁺ (100%).

### e) 7-(2-Amino-ethoxy)-1 ethyl-4-oxo-1,4-dihydro-quinoline-3-carboxylic acid sodium salt

Intermediate 5d (0.225 g, 0.74 mmol) was dissolved in THF (10 mL) and 1,4-dioxane (10 mL), then aqueous sodium hydroxide (0.2N, 3.7 mL, 0.74 mmol) was added, and the mixture stirred for 18 h. Solid carbon dioxide was then added and the solution evaporated to dryness under reduced pressure to give the title compound as a pale yellow solid (0.212 g, 96%); ESMS m/z 277 [M+H]⁺ for free acid (100%).

### Intermediate 6: 6-(2-Amino-ethylsulfanyl)-1-ethyl-4-oxo-1,4-dihydro-quinoline-3-carboxylic acid trifluoroacetate salt

### a) 6-Bromo-1-ethyl-4-oxo-1,4-hydroquinolin-3-carboxylic acid ethyl ester

A mixture of potassium carbonate (2.95 g, 21.2 mmol) and 6-bromoquinolone-3-carboxylic acid in dimethylformamide (25 mL) was heated to 40° C. under argon for 10 minutes and iodoethane (3.4 mL, 42.4 mmol) was added. After 14 h the mixture was cooled and the DMF evaporated. The residue was treated with water (40 mL), cooled to 5° C. and filtered under vacuum. The resultant cream coloured solid was dried under vacuum to yield the title compound; ¹H NMR δ [(CD₃)₂SO] 1.41 (3H, t, J=7.1 Hz), 1.54 (3H, J=7.2 Hz), 4.24 (2H, q, J=7.2 Hz), 4.40 (2H, q, J=7.1 Hz), 7.34 (1H, d, J=9 Hz), 7.76 (1H, dd, J=2.4 & 9 Hz), 8.65 (1H, d, J=2.4 Hz), 8.49 (1H, s).

### b) 6-(2-t-Butoxycarbonylaminoethylsulfanyl)-1-ethyl-4-oxo-1,4-dihydro-quinoline-3-carboxylic acid ethyl ester

A mixture of N-Boc-cysteinamine (0.35 g, 2 mmol), Intermediate 6a (0.32 g, 1 mmol) and potassium carbonate (0.28 g, 2 mmol) was heated in DMSO (10 mL) for 16 h at 90° C. After chromatography over silica gel eluting with dichloromethane containing an increasing concentration of methanol/ammonium hydroxide the title compound was obtained as a white solid; ESMS m/z 421 [M+H]⁺ (100%).

### c) 6-(2-t-Butoxycarbonylaminoethylsulfanyl)-1-ethyl-4-oxo-1,4-dihydro-quinoline-3-carboxylic acid sodium salt

To a solution of Intermediate 6b (0.11 g, 0.27 mmol) in THF (2 mL) was added 2M sodium hydroxide (0.13 mL, 0.27 mmol). After stirring for 16 h at room temperature the mixture was saturated with carbon dioxide and the solvent evaporated. The residue was treated with methanol (10 mL), filtered and the solvent evaporated to yield the title compound as a pale yellow solid; ESMS m/z 393 [M+H]⁺ (25%).

### d) 6-(2-Amino-ethylsulfanyl)-1-ethyl-4-oxo-1,4-dihydro-quinoline-3-carboxylic acid trifluoroacetate salt

To Intermediate 6c (0.068 g, 0.17 mmol) was added trifluoroacetic acid (1 mL). After 1 h the solvent was evaporated to yield a green gum; ¹H NMR δ [(CD₃)₂SO] 1.54 (3H, t, J=7.2 Hz), 3.20 (2H, q, J=6.8 Hz), 3.38 (2H, t, J=6.8 Hz), 4.56 (2H, q, J=7.2 Hz), 7.98-7.90 (2H, m), 8.40 (1H, d, J=2.0 Hz), 8.94 (1H, s).

### Intermediate 7: 6-(2-Aminoethyl-sulfanyl)-1-ethyl-4-oxo-1,4-dihydro-quinoline-3-carboxylic acid isopropyl ester

### a) 6-(2-t-Butoxycarbonylaminoethylsulfanyl)-1-ethyl-4-oxo-1,4-dihydro-quinoline-3-carboxylic acid isopropyl ester

A mixture of Intermediate 6c (0.414 g, 1.0 mmol), potassium carbonate (0.35 g, 2.5 mmol) and 2-iodopropane (0.1 mL, 1.0 mmol) in DMF was heated at 80° C. After 5 h the reaction mixture was cooled and diethyl ether added. The resultant biphasic mixture was washed with water and the organic layer separated and washed with 2M aqueous sodium hydroxide. The organic phase was dried and evaporated to yield the crude compound. Chromatography over silica gel eluting with dichloromethane containing an increasing concentration of ammonium hydroxide gave the title compound as a white solid; ¹H NMR δ (CDCl₃) 1.39(6H, d, J=6.2 Hz), 1.43(9H, s), 1.53(3H, t, J=7.2 Hz), 3.13 (2H, t, J=6.3 Hz), 3.34 (2H, t, J=6.3 Hz), 4.24 (2H, q, J=7.2 Hz), 5.0 (1H, br. s), 5.2-5.3 (1H, m), 7.40 (1H, d, J=8.9 Hz), 7.62 (1H, dd, J=8.9 & 2.3 Hz), 8.40 (1H, d, J=2.3 Hz), 8.41 (1H, s).

### b) 6-(2-Aminoethyl-sulfanyl)-1-ethyl-4-oxo-1,4-dihydro-quinoline-3-carboxylic acid isopropyl ester

Using an identical procedure to that described for the preparation of Intermediate 6d, Intermediate 7a (0.14 g, 0.32 mmol) and trifluoroacetic acid gave, after chromatography over silica gel eluting with dichloromethane containing an increasing concentration of methanol/ammonium hydroxide, the title compound; ¹H NMR δ (CDCl₃+D₂O) 1.39 (6H, d, J=6.3 Hz), 1.54 (3H, t, J=7.2 Hz), 2.94 (2H, t, J=7.2 Hz), 3.13 (2H, t, J=7.2 Hz), 4.24 (2H, q, J=7.2 Hz), 5.2-5.3 (1H, m), 7.37 (1H, d, J=8.9 Hz), 7.62 (1H, dd, J=8.9 & 2.4 Hz), 8.42 (1H, d, J=2.3 Hz), 8.43 (1H, s).

### Intermediate 8: 2′-O-Acetyl-4″-O-propenoyl-azithromycin11,12-carbonate

A solution of 2′-O-acetyl-azithromycin11,12-carbonate (10.9 g) in toluene (300 mL) was stirred at room temperature under argon atmosphere. To this solution TEA (12.66 mL) and 3-chloropropionyl chloride (1.94 mL) were added in two portions over a period of 10 minutes. After 20 minutes the solution was diluted with a saturated aqueous solution of NaHCO₃ (300 mL) and extracted with toluene (4×80 mL). The collected organic phase was dried, filtered and concentrated under reduced pressure affording the title compound (11.0 g); MS; m/z (ES): 872 [MH]⁺.

### Intermediate 9: 4″-O-Propenoyl-azithromycin11,12-carbonate

A solution of Intermediate 8 (11.0 g) in MeOH (200 mL) was stirred at room temperature for 48 h. The solvent was evaporated under reduced pressure affording the title compound (9.81 g); MS; m/z (ES): 829.1 [MH]⁺; ¹H-NMR (500 MHz,) δ: 6.45 (d, 1H), 6.17 (dd, 1H), 5.87 (d, 1H), 5.11 (d, 1H), 4.88 (dd, 1H), 4.77 (d, 1H), 4.53 (d, 1H), 4.47-4.40 (m, 3H), 3.72 (m, 1H), 3.60 (d, 1H), 3.33 (s, 3H), 3.25 (dd, 1H), 2.87-2.85 (m, 2H), 2.58 (m, 1H), 2.44-2.38 (m, 2H), 2.32 (s, 6H), 2.21 (s, 3H), 2.06 (m, 1H), 2.00 (m, 1H), 1.92 (m, 1H), 1.84 (m, 1H), 170-1.56 (m, 4H), 1.45 (s, 3H), 1.40 (dd, 1H), 1.29 (s, 3H), 1.25 (m, 1H), 1.22 (d, 3H), 1.18 (d, 6H), 1.12 (s, 3H), 108-1.06 (2d, 6H), 0.93 (m, 6H).

### Intermediate 10: 4″-O-Propenoyl-azithromycin

To a solution of Intermediate 9 (1.3 g) in acetonitrile (50 mL), a saturated aqueous solution of potassium carbonate (30 mL) was added at room temperature. The resulting mixture was heated to 70° C. for 8 h. The mixture was then diluted with water (100 mL), extracted with EtOAc (4×30 mL). The collected organic phase was dried, filtered and concentrated under reduced pressure. The crude product was purified by flash chromatography (eluent: DCM/MeOH/NH₃ 90/9/0.5) affording the title compound (530 mg); MS; m/z (ES): 804 [MH]⁺.

### Intermediate 11: 2′-O-Acetyl-4″-O-propenoyl-6-O-methyl-erythromycin A

To a solution of 2′-O-acetyl-6-O-methyl-erythromycin A (1.1 g) in DCM (20 mL) pyridine (1.7 mL) and acryloylchloride (1.1 mL) were added at 0° C. After 2 h a further addition of pyridine (1.7 mL) and of acryloylchloride (1.1 mL) was performed. The reaction mixture was quenched with a saturated solution of NH₄Cl (10 mL) and extracted with DCM (3×20 mL). The organic phase washed with a saturated solution of NaHCO₃ (10 mL), water (10 mL), dried over Na₂SO₄, filtered and evaporated under reduced pressure. The crude product was purified by flash-chromatography (DCM/MeOH/NH₃95/5/0.5) affording the title compound (470 mg); ESMS m/z 844 [M+H]⁺.

### Intermediate 12: 4″-O-Propenoyl-6-O-methyl-erythromycin A

Intermediate 11 (1.82 g. mmol) was dissolved in MeOH (100 mL) and stirred at 60° C. for 4 h, then at room temperature for 16 h. The solvent was evaporated under reduced pressure and the crude product was purified by flash chromatography (eluent: MeOH/DCM/NH₄OH 5/90/0) affording the title compound (1.4 g); MS; m/z (ES): 802 [MH]⁺; ¹H-NMR (500 MHz) δ: 6.44 (d, 1H), 6.13 (dd, 1H), 5.89 (d, 1H), 5.07 (d, 1H), 5.00 (d 1H), 4.75 (d, 1H), 4.60 (d, 1H), 4.38 (m, 1H), 3.97 (s, 1H), 3.80-3.73 (m, 2H), 3.66 (d, 1H), 3.46 (s, 1H), 3.32 (s, 3H), 3.21-3.18 (m, 2H), 3.04 (s, 3H), 3.00 (m, 1H), 2.92 (m, 1H). 2.56 (m, 2H), 2.43 (d, 1H), 2.31 (s, 6H); ¹³C-NMR (75 MHz) δ: 221.0; 175.7; 165.8; 131.5; 128.0; 102.1; 96.0; 80.5, 78.8, 78.3; 78.0; 76.6; 74.3, 72.7; 71.1; 69.1; 67.8; 65.3; 63.2: 50.7; 49.5; 45.3; 44.9; 40.3; 39.2; 38.8; 37.2; 35.2; 28.9; 21.7, 21.1; 19.7, 18.3, 18.0, 15.9; 12.3; 10.6; 9.1.

### Intermediate 13: 2′-O-Acetyl-6-O-methyl-11-desoxy-11(R)-amino-erythromycin A11,12-carbamate

To a solution of 6-O-methyl-11-desoxy-11-(R)-amino-erythromycin A11,12 carbamate (W. R. Baker et al., J. Org. Chem., 1988, 53(10), 2340-5) in DCM (50 mL) was added NaHCO₃ (478 mg) at room temperature. To this solution Ac₂O (0.153 mL) was added and stirred overnight. To this mixture brine (50 mL) and water (20 mL) were added. The organic layer was separated, washed with brine (20 mL), dried, filtered and evaporated under reduced pressure, affording the title compound (1.2 g); MS; m/z (ES): 816.2 [MH]⁺.

### Intermediate 14: 2′-O-Acetyl-4″-O-propenoyl-6-O-methyl-11-desoxy-11-(R)-amino-erythromycin A11,12-carbamate

Intermediate 13 was dissolved in toluene (50 mL) and the solvent was evaporated. This was performed 2 times. After that the residue was again dissolved in toluene (45 mL) and stirred under argon. To this solution TEA (1.8 mL) and 3-chloropropionylchloride (0.40 mL) (in 3 portions in a period of 20 minutes) were added. 20 min later a saturated aqueous solution of NaHCO₃ (50 mL) was added. The aqueous solution was extracted with toluene (3×50 mL), the combined organic solution dried over K₂CO₃ and the solvent removed under reduced pressure affording the title compound (1.04 g); MS; m/z (ES): 870.1 [MH]⁺.

### Intermediate 15

### 7-chloro-1-cyclopropyl-6-(2-hydroxy-ethylamino)-4-oxo-1,4-dihydro-quinoline-3-carboxylic acid (A) and

### 1-Cyclopropyl-6-fluoro-7-(2-hydroxy-ethylamino)-4-oxo-1,4-dihydro-quinoline-3-carboxylic acid (B)

To a solution of ethanolamine (55.5 mL) in N-methylpyrrolidinone (500 mL) at 95° C., 7-chloro-1-cyclopropyl-6-fluoro-4-oxo-1,4-dihydro-quinoline-3-carboxylic acid (50.0 g) was slowly added under vigorous stirring. The temperature was increased to 105° C. and the reaction mixture was stirred at this temperature for 22 hours. The reaction mixture was cooled to about 60° C. and poured into MeOH (800 mL). This mixture was stirred in an ice bath and the precipitate was filtered off and dried affording a mixture of Intermediate 15A and Intermediate 15B (49 g) in a 1:1 ratio.

Intermediate 15A: MS; m/z (ES): 322.99 [MH]⁺

Intermediate 15B: MS; m/z (ES): 307.02 [MH]⁺

### Intermediate 16

### 7-chloro-6-[2-(2-cyano-ethoxy)-ethylamino]-1-cyclopropyl-4-oxo-1,4-dihydro-quinoline-3-carboxylic acid (A) and

### 7-[2-(2-cyano-ethoxy)-ethylamino]-1-cyclopropyl-6-fluoro-4-oxo-1,4-dihydro-quinoline-3-carboxylic acid (B)

A solution of a mixture of Intermediate 15A and Intermediate 15B (14 g) in acrylonitrile (140 mL) and DBU (14 mL) was stirred at 70° C. for 16 hours. The solvent was evaporated and the residue dissolved in i-PrOH (50 mL). Water (50 mL) was added and the pH value adjusted to 4. The precipitate was filtered and then triturated with methanol. After filtration, 5.35 g of pure Intermediate 16A was obtained. The mother liquor was left overnight at 4° C. and 4.4 g of Intermediate 16B precipitated.

Intermediate 16A: ¹H-NMR (500 MHz, DMSO-d6) δ: 8.56 (s, 1H), 8.23 (s, 1H), 7.40 (s, 1H), 5.93 (t, NH), 3.83 (qv, 1H), 3.72 (t, 2H), 3.67 (t, 2H), 3.46 (q, 2H), 2.79 (t, 2H), 1.30 (q, 2H), 1.18 (q, 2H). ¹³C-NMR (75 MHz, DMSO-d6) δ: 176.52, 166.09, 145.72, 142.72, 132.17, 126.37, 125.38, 119.15, 118.99, 106.14, 102.76, 67.93, 65.05, 42.40, 35.77, 18.01, 7.32. MS; m/z (ES): 376.02 [MH]⁺

Intermediate 16B: ¹H-NMR (500 MHz, DMSO-d6) δ: 8.55 (s, 1H), 7.76 (d, 1H), 7.22 (d, 1H), 3.74 (t, 2H+1H), 3.67 (t, 2H), 3.52 (q, 2H), 2.78 (t, 2H), 1.31 (m, 2H), 1.18 (m, 2H). ¹³C-NMR (75 MHz, DMSO-d6) δ: 175.80, 166.20, 148.12, 146.89, 142.55, 140.30, 119.22, 108.79, 106.10, 96.68, 68.29, 65.17, 42.06, 35.70, 17.99, 7.48. MS; m/z (ES): 360.04 [MH]⁺

### Intermediate 17

### 6-[2-(2-Carboxy-ethoxy)-ethylamino]-7-chloro-1-cyclopropyl-4-oxo-1,4-dihydro-quinoline-3-carboxylic acid

A solution of Intermediate 16A (4.7 g) in 60 mL conc. H₂SO₄ and 60 mL H₂O was stirred for 20 hours at 75° C. The reaction mixture was poured into water (150 mL) and the pH value was adjusted to 2. Filtration of the precipitate obtained yielded pure Intermediate 17 (3.07 g); ¹H-NMR (500 MHz, DMSO-d6) δ: 8.56 (s, 1H), 8.23 (s, 1H), 7.39 (s, 1H), 3.82 (m, 1H), 3.66 (q, 2H+2H), 3.42 (t, 2H), 2.49 (t, 2H), 1.30 (q. 2H), 1.17 (m, 2H); ¹³C-NMR (75 MHz, DMSO-d6) δ: 178.70, 174.73, 168.28, 147.89, 144.93, 134.34, 128.55, 127.56, 121.15, 118.99, 108.32, 104.90, 69.98, 68.16, 44.59, 37.95, 36.74, 9.50; MS; m/z (ES): 395.05 [MH]⁺.

### Intermediate 18

### 7-chloro-1-cyclopropyl-6-(2-hydroxy-ethoxy)-4-oxo-1,4-dihydro-quinoline-3-carboxylic acid (A) and

### 1-cyclopropyl-6-fluoro-7-(2-hydroxyethoxy)-4-oxo-1,4-dihydro-quinoline-3-carboxylic acid (B)

To a mixture of DMSO (5 mL) and ethyleneglycol (6 mL), KO^{t}Bu (1.6 g, 14.23 mmol) was added portionwise over 10 min, and then heated to 90° C. To the mixture, 7-chloro-1-cyclopropyl-6-fluoro-4-oxo-1,4-dihydro-quinoline-3-carboxylic acid (1.0 g) was added portionwise over 20 min, the temperature was increased to 105° C. and the mixture was stirred for 6 h. Water (30 mL) was added to the reaction solution and the pH of the solution was adjusted to pH=5. The resulting solution was left in the refrigerator overnight. The precipitate obtained was filtered, washed with cold water, and dried affording a 2:1 mixture of Intermediate 18A and Intermediate 18B (1.0 g).

Part of the crude product (700 mg) was dissolved in EtOH (15 mL) by heating to the reflux. The resulting solution was cooled to 30° C. and a first precipitation occurred. The precipitate was filtered, washed with cold EtOH and dried under reduced pressure. Intermediate 18A (204 mg) was obtained as a white solid; ¹H-NMR (500 MHz, DMSO-d6) δ: 15.06 (s, 1H), 8.71 (s, 1H), 8.40 (s, 1H), 7.86 (s, 1H), 4.97 (t, 1H), 4.25 (t, 2H), 3.87 (m, 1H), 3.82 (q, 2H), 1.32 (m, 2H), 1.20 (m, 2H); ¹³C-NMR (75 MHz, DMSO-d6) δ: 176.61, 165.67, 152.47, 147.54, 135.34, 129.48, 124.95, 120.02, 106.90, 106.66, 71.22, 59.15, 35.99, 7.46; MS; m/z (ES): [MH]⁺.

### Intermediate 19: 7-chloro-6-[2-(2-cyano-ethoxy)-ethoxy]-1-cyclopropyl-4-oxo-1,4-dihydro-quinoline-3-carboxylic acid

To a suspension of Intermediate 18A (2 g) in acrylonitrile (40 mL) was added DBU (2.3 mL). The reaction mixture was stirred at 80° C. for 24 h. The acrylonitrile was evaporated under reduced pressure. Isopropanol (30 mL) was added to the residue and the pH of the solution was adjusted to pH=5 by adding 2M HCl, during which the product precipitated. The precipitate was filtered, washed with water, and dried affording Intermediate 19 (1.7 g) as a white solid; ¹H-NMR (500 MHz, DMSO-6) δ: 8.68 (s, 1H), 8.38 (s, 1H), 7.84 (s, 1H), 4.38 (t, 2H), 3.91 (t, 2H), 3.86 (m, 1H), 3.75 (t, 2H), 2.79 (t, 2H), 1.32 (m, 2H), 1.20 (m, 2H); ¹³C-NMR (75 MHz, DMSO-d6) δ: 176.63, 165.65, 152.18, 147.61, 135.50, 129.44, 124.97, 120.04, 119.11, 106.96, 106.80, 69.02, 68.30, 65.49, 35.99, 18.06, 7.46; MS; m/z (ES): 377.03 [MH]⁺.

### Intermediate 20: 6-[2-(2-Carboxy-ethoxy)-ethoxy]-7-chloro-1-cyclopropyl-4-oxo-1,4-dihydro-quinoline-3-carboxylic acid

A solution of Intermediate 19 (1.10 g) in a mixture of conc. H₂SO₄ (10 mL) and H₂O (20 mL) was stirred at 75° C. for 24 h. The pH of the reaction mixture was adjusted to 0.2 with 40% NaOH, during which the product precipitated. The precipitate was filtered, washed with water, and dried affording Intermediate 20 (0.8 g) as a white solid; ¹H-NMR (300 MHz, DMSO-d6) δ: 15.0 (s, 1H), 11.8 (s, 1H), 8.69 (s, 1H), 8.38 (s, 1H), 7.85 (s, 1H), 4.35 (m, 2H), 3.91-3.82 (m, 3H), 3.74 (dt, 2H), 2.49 (m, 2H), 1.31 (m, 2H), 1.19 (m, 2H); MS; m/z (ES): 396.02 [MH]⁺.

### Intermediate 21: 7-[2-(2-Carboxy-ethoxy)-ethylamino]-1-cyclopropyl-6-fluoro-4-oxo-1,4-dihydro-quinoline-3-carboxylic acid

A solution of Intermediate 16B (4.5 g) in 75 mL conc. H₂SO₄ and 74 mL H₂O was stirred for 18 hours. The reaction was poured into water and the pH value was adjusted to 3. Filtration yielded the title compound (2.3 g) as an white solid; ¹H-NMR (500 MHz, DMSO-d6) δ: 8.56 (s, 1H), 7.76 (d, 1H), 7.21 (d, 1H), 3.75 (m, 1H), 3.67 (q, 2H+2H). 3.47 (q, 2H), 2.47 (t, 2H), 1.31 (q, 2H), 1.16 (m, 2H); ¹³C-NMR (75 MHz, DMSO-d6) δ: 175.83, 172.57, 166.22, 148.11, 146.91, 142.58, 140.30, 113.71, 108.63, 106.10, 96.72, 68.27, 66.13, 42.13, 35.69, 34.58, 7.48. MS; m/z (ES): 379.00 [MH]⁺.

### Intermediate 22: 2′-O-Acetyl-11-O-methyl-azithromycin

To a solution of 11-O-methyl-azithromycin (0.517 g) in EtOAc (15 mL) at room temperature was added acetic anhydride (100 μL) and the mixture was stirred for 12 hours at r.t. Saturated aqueous NaHCO₃ (30 mL) was added to the reaction mixture and the layers were separated. The water layer was extracted with EtOAc (2×20 mL). The combined organic layers were dried over Na₂SO₄ and evaporated yielding the crude product (0.460 g).

### Intermediate 23: 1-Cyclopropyl-6-fluoro-7-(2-hydroxy-ethylamino)-8-methoxy-4-oxo-dihydro-quinoline-3-carboxylic acid

To a suspension of 1-cyclopropyl-6,7-difluoro-8-methoxy-4-oxo-1,4-dihydroquinoline-3-carboxylic acid (10 g) in DMSO (80 mL), ethanolamine (20.68 mL) was added. The reaction mixture was stirred at 90° C. for 1.5 hours. The pH of mixture was then adjusted to 4.5 and the product was precipitated. The precipitate was filtered off yielding 10.45 g of the title compound (according to LC-MS 100% pure compound). MS (ES+)M/Z: [MH]⁺=337.32.

### Intermediate 24: 7-[2-(2-Cyano-ethoxy)-ethylamino]-1-cyclopropyl-6-fluoro-8-methoxy-4-oxo-1,4-dihydro-quinoline-3-carboxylic acid

To a suspension of Intermediate 23 (4 g) in acrylonitrile (50 mL), DBU (4 mL) was added and the reaction mixture was stirred at 80° C. under N₂ for 5 h. The acrylonitrile was evaporated under reduced pressure. The residue was dissolved in acetone, the pH was adjusted to pH=2 and the solution was then cooled in the fridge. The product was precipitated and filtrated off yielding 3.45 g of crude title compound (according to LC-MS 94.4% pure compound). MS (ES+)m/z: [MH]⁺=390.38.

### Intermediate 25: 7-[2-(2-Carboxy-ethoxy)ethylamino]-1-cyclopropyl-6-fluoro-8-methoxy-4-oxo-1,4-dihydro-quinoline-3-carboxylic acid

A suspension of Intermediate 24 (1.5 g) in H₂O:H₂SO₄ (1:1) (3 mL) was stirred for 24 h at 75° C. The pH of the mixture was adjusted to 4.5 and the mixture was extracted with 3×20 mL DCM. The organic layers were washed with brine, dried over Na₂SO₄, filtered and the DCM was evaporated under reduced pressure affording 1.4 g of product. The product was precipitated from EtOAc:diisopropyl-ether yielding 1.1 g of the title compound. MS (ES+)m/z: [MH]⁺=408.

### Intermediate 26: 9E-Methoximino erythromycin A

To a suspension of sodium acetate (500 mg, 6.09 mmol) in dry methanol (40 mL) heated to 50° C., was added erythromycin A (4 g, 5.4 mmol) and methoxylamine hydrochloride (500 mg, 5.98 mmol). The solution was then stirred at room temperature. After 4 hours another 500 mg portion of methoxylamine hydrochloride was added and after another 14 hours one more portion of 500 mg methoxylamine hydrochloride was added to the mixture. The reaction was monitored by TLC (E: DCM-MeOH—NH₃/90-9-1.5). The solvent was removed in vacuo. The residue was partitioned between dichloromethane (100 mL) and saturated aqueous sodium carbonate. The organic layer washed with brine, dried and DCM evaporated. The residue was dissolved in acetone and cooled in a refrigerator overnight. After 24 h, colorless crystals formed. The crystals were filtered off giving 730 mg of the title compound. ESMS m/z 763 [MH⁺].

### Intermediate 27: 2′-O-Acetyl-(9E)-O-methoxilmino erythromycin A

To a solution of Intermediate 26 (700 mg) in CH₂Cl₂ (15 mL) was added acetic anhydride (130 μL, 1.5 eq) and NaHCO₃ (269 mg, 3.5 eq). After stirring at room temperature for 2 h, 15 mL of water was added and the layers were separated. The organic layer washed with brine, dried and evaporated to yield the title compound (710 mg) as a white solid. ESMS m/z 805 [MH⁺].

### Intermediate 28: 2′-O-Acetyl-(9E)-O-(2-propyl)oximino erythromycin A

To a solution of 9(E)-O-(2-propyl)oximino erythromycin A (800 mg, 1.01 mmol) in CH₂Cl₂ (15 mL) was added acetic anhydride (143 μL, 1.5 mmol) and NaHCO₃ (297 mg, 3.5 mmol). After stirring at room temperature for 2 h, 15 mL of water was added and the layers were separated. The organic layer washed with brine, dried and evaporated to yield the title product (758 mg) as a white solid. ESMS m/z 834 [MH⁺].

### Intermediate 29: 4″-O-Glycyl-2′-O-acetyl-azithromycin-11,12-cyclic carbonate

Method A (EDC Mediated Coupling)

At 25° C., a solution of N-t-F-mocglycine (87.5 mg, 0.5 mmoL), EDC.HCl (127.8 mg, 0.67 mmoL) and 4-dimethylaminopyridine (20.4 mg, 0.17 mmoL) in 600 μL of methylene chloride was treated with 2′-O-acetyl-azithromycin-11,12-carbonate (91.2 mg, 0.1 mmol). The mixture was stirred for 3 days. It was then filtered, and the filtrate was evaporated under reduced pressure to furnish a crude product. LC/MS analysis of the crude reaction mixture at 50% conversion of starting material showed the major product along with unreacted starting material. The crude material was subjected to methanolysis at room temperature and then chromatographed on silica gel (gradient elution, 2% MeOH-0.25% NH₄OH in methylene chloride to 4% MeOH-0.5 NH₄OH in methylene chloride) to afford 16.6 mg (20%) of deprotected product. MS (m/z) 832 (M⁺+1).

Method B (DCC—N-hydroxysuccinimide Procedure)

A solution of N-t-Fmocglycine (87.5 mg, 0.5 mmoL) in anhydrous methylene chloride (5.0 mL) was treated with N-hydroxysuccinimide (61 mg, 0.53 mmoL) and dicyclohexylcarbodiimide (DCC) (109 mg, 0.53 mmoL), and the mixture was stirred for 2 h under nitrogen. 2′-O-Acetyl-azithromycin-11,12-carbonate was added (91.2 mg, 0.1 mmoL), and the reaction mixture was stirred for an additional 12 h. The reaction mixture was suction-filtered, and the filtrate was concentrated. The residue was purified by silica gel chromatography eluting with E1 system, yielding the title compound (25 mg, 30%) as a white solid.

Method C (HOAt—EDC Coupling)

A mixture of N-Fmocglycine (87.5 mg, 0.5 mmoL) and 2′-O-acetyl-azithromycin-11,12-carbonate (91.2 mg, 0.1 mmoL) were azeotroped with benzene (3×3 mL) and dried further under vacuum for 2 h prior to reaction. The mixture was cooled at 0° C. under nitrogen and dry methylene chloride was added. 1-Hydroxy-7-azabenzotriazole (1 mmoL) was then added, followed by EDC (0.95 mmoL). The mixture was stirred at 0° C. for 5 h, and 12 h at room temperature, before it was diluted with EtOAc (5 mL) and washed with sat. aq. NaHCO₃ solution (3 mL), water (3 mL) and brine (3 mL). The organic layer was dried (MgSO₄) and concentrated in vacuo to give the crude product, which was purified by silica-gel chromatography (E1 system) to afford the title compound (20.8 mg, 25%) as a white solid.

Method D (HOBt—EDC Coupling)

N-Fmocglycine (553.6 mg, 3.16 mmoL) was dissolved in methylene chloride at room temperature. HOBt (513.0 mg, 3.8 mmoL) was added in one portion followed by EDC (729.2 mg, 3.80 mmoL). The mixture was stirred at room temperature, and the reaction progress was monitored by HPLC until all of the acid was converted to the activated ester/amide mixture. The resulting mixture was then slowly added to a solution of 2′-O-acetyl-azithromycin-11,12-carbonate (970 mg, 1.58 mmoL) in methylene chloride (3 mL) while the temperature was maintained at 0-10° C. The reaction was usually complete upon overnight stirring. Water (20 mL) was added. The aqueous mixture was extracted with EtOAc followed by a carbonate wash of the organic layer to remove HOBt. Removal of the solvents under reduced pressure yielded the crude reaction mixture, which was, purified by silica-gel chromatography (E1 system) to afford the title compound (23.3 mg, 28%) as a white solid.

### Intermediate 30: 6-O-Methyl-(9E)-O-ethyloximino erythromycin A

Powdered potassium hydroxide (0.5 g, 1 eq) was added to a mixture of 6-O-methyl-erythromycin A 9(E)-oxime (0.5 g, 1 eq), tetrabutylammonium iodide (0.125 g, 0.05 eq), sodium iodide (0.15 g, 0.15 eq) and 1.5 eq of 1-bromoethane (0.75 mL) in 50 mL of THF at room temperature with stirring for 7 hours. The solvent was evaporated under reduced pressure and to the residue was added saturated aqueous sodium hydrogen carbonate solution (50 mL). The mixture was extracted with DCM. The organic layers were collected and washed with water and saturated brine, dried over K₂CO₃, filtered, and evaporated to afford 3.666 g of the title compound as a white solid. ESMS m/z 791.47 [MH⁺].

### Intermediate 31: 2′-O-Acetyl-6-O-methyl-9(E)-O-ethyloxlmino erythromycin A

To a solution of Intermediate 30 (3.11 g; 3.9 mmol) in CH₂Cl₂ (90 mL), was added acetic anhydride (0.583 mL, 1.5 eq) and NaHCO₃ (1.17 g, 3.5 eq). After stirring at room temperature for 5 hours, 90 mL of water was added to the reaction mixture, the pH was adjusted to 9 with 1N NaOH and the layers were separated. The organic layer was washed with brine and H₂O, dried and evaporated to yield the title product as a white solid (2.7 g). ESMS m/z 833.6 [MH⁺].

### Example 1

### 4″-O-{3-[2-(3-Carboxy-1-ethyl-4-oxo-1,4-dihydro-7-quinolinylsulfanyl)ethylamino]propionyl}-azithromycin

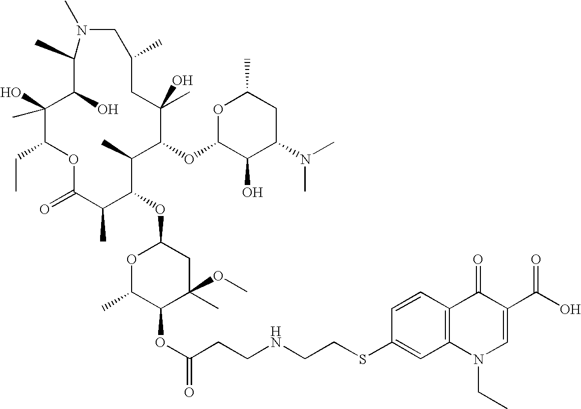

Intermediate 1 (0.031 g) and Intermediate 10 (0.063 g, 0.075 mmol) were mixed in DMSO (0.5 mL) containing water (1 drop) and triethylamine (0.021 mL). The container was flushed with argon, sealed, and heated with stirring at 80° C. for 24 h. The reaction mixture was diluted with methanol (0.5 mL) and injected onto a preparative reverse phase HPLC. The product was desalted by chromatography on silica gel (0.5 g), eluting with 5-15% methanolic ammonia (2M) in dichloromethane, to give the title compound as a white solid (0.045 g, 55%); ESMS m/z 1093 [M−H]⁻ (100%).

### Example 2

### 4″-O-{3-[2-(3-Carboxy-1-ethyl-4-oxo-1,4-dihydro-7-quinolinylsulfanyl)ethylamino]propionyl}-azithromycin-11,12-carbonate

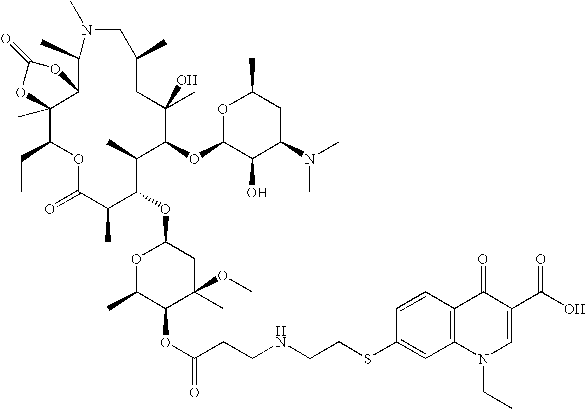

Intermediate 1 (0.031 g) and Intermediate 9 (0.065 g, 0.075 mmol) were mixed in DMSO (0.5 mL) containing water (1 drop) and triethylamine (0.021 mL). The container was flushed with argon, sealed, and heated with stirring at 80° C. for 24 h. Further triethylamine (0.01 mL) was added and the reaction heated at 80° C. for a further 28 h. The reaction mixture was diluted with methanol (0.5 mL) and injected onto a preparative reverse phase HPLC. The product was desalted by chromatography on silica gel (0.5 g), eluting with 5-15% methanolic ammonia (2M) in dichloromethane to give the title product as a white solid (0.012 g, 14%); ESMS m/z 1119 [M−H]⁻ (80%), 1165 [M+HCO₂]⁻ (100%).

### Example 3

### 4″-O-{3-[2-(3-Carboxy-1-ethyl-4-oxo-1,4-dihydro-7-quinolinylsulfanyl)ethylamino]propionyl}-6-O-methyl-erythromycin A

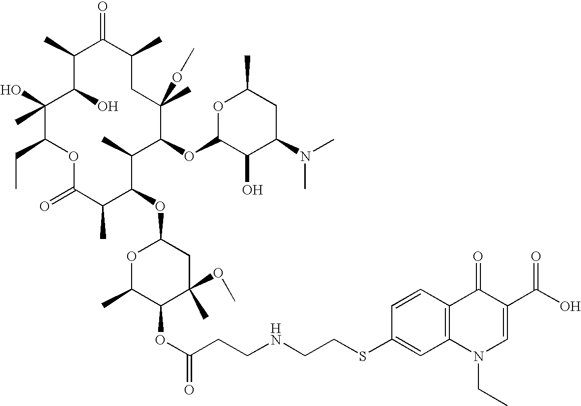

### a) 2′-O-Acetyl-4″-O-{3-[2-(3-Carboxyl-ethyl-4-oxo-1,4-dihydro-7-quinolinylsulfanyl)ethylamino]propionyl}-6-O-methyl-erythromycin A

Intermediate 1 (0.031 g) and Intermediate 11 (0.063 g, 0.075 mmol) were mixed in DMSO (0.5 mL) containing water (1 drop) and triethylamine (0.021 mL). The container was flushed with argon, sealed, and heated with stirring at 80° C. for 24 h. Further triethylamine (0.01 mL) was added and the reaction heated at 80° C. for a further 28 h. The reaction mixture was diluted with methanol (0.5 mL) and injected onto a preparative reverse phase HPLC. The product was desalted by chromatography on silica gel (0.5 g), eluting with 5-15% methanolic ammonia (2M) in dichloromethane obtained as a white solid (0.030 g, 36%); ESMS m/z 1134 [M−H]⁻ (60%), 1180 [M+HCO₂]⁻ (100%).

### b) 4″-O-{3-[2-(3-Carboxy-1-ethyl-4-oxo-1,4-dihydro-7-quinolinylsulfanyl)ethylamino]propionyl}-6-O-methyl-erythromycin A

Example 3a (0.03 g) in methanol (10 mL) was heated under argon at 50° C. for 48 h. The solvent was removed by evaporation under reduced pressure to give the title product as a white solid (0.017 g, 59%); ESMS m/z 1092 [M−H]⁻ (50%), 1138 [M+HCO₂]⁻ (100%).

### Example 4

### 4″-O-{3-[2-(3-Carboxy-1-ethyl-4-oxo-1,4-dihydro-[1,8]naphthyridin-6-ylsulfanyl)ethylamino]propionyl}-azithromycin tris trifluoroacetate salt

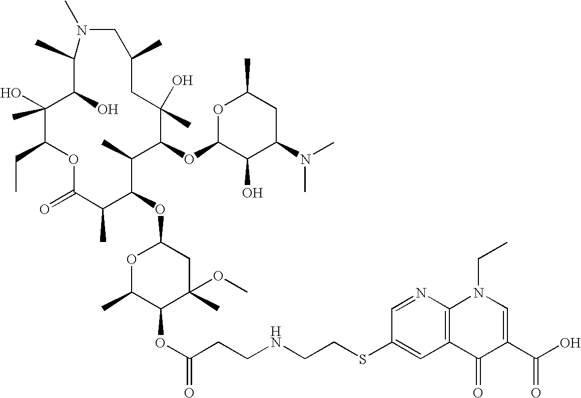

Intermediate 2 (0.041 g, 0.1 mmol) and Intermediate 10 (0.08 g, 0.1 mmol) were mixed in DMSO (0.5 mL) containing water (1 drop) and triethylamine (0.042 mL, 0.3 mmol). The container was flushed with argon and heated with stirring at 80° C. for 87 h. The reaction mixture was diluted with methanol (0.5 mL) and injected onto a preparative reverse phase HPLC. The product was obtained as a white solid (0.045 g, 31%); ESMS m/z 1096 [M+H]⁺ (10%), 548 [M+2H]²⁺ (100%).

### Example 5

### 4″-O-{3-[2-(3-Carboxy-1-ethyl-4-oxo-1,4-dihydro-[1,8]naphthyridin-6-ylsulfanyl)ethylamino]propionyl}-6-O-methyl-erythromycin A monoformate salt

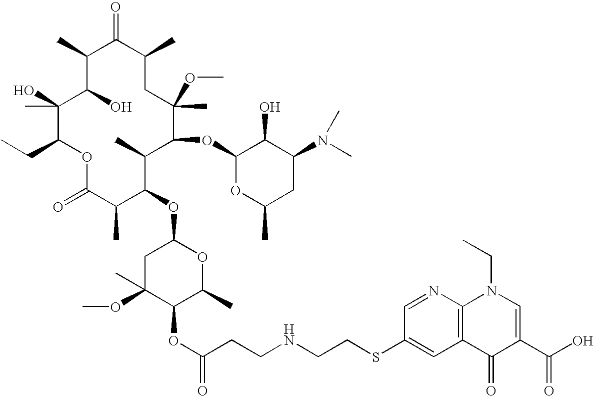

Intermediate 2 (0.041 g, 0.1 mmol) and Intermediate 12 (0.073 g, 0.091 mmol) were mixed in DMSO (0.5 mL) containing water (1 drop) and triethylamine (0.038 mL, 0.273 mmol). The container was flushed with argon and heated with stirring at 80° C. Further Intermediate 2 (2×0.01 g, 0.025 mmol) was added after 3 and 5 days, along with more triethylamine (2×0.038 mL). After 17 days the reaction mixture was filtered, diluted with acetonitrile (0.5 mL), and then injected onto a preparative reverse phase HPLC. The product was obtained as an off-white solid (0.0095 g, 9%); ESMS m/z 1095 [M+H]⁺ (100%).

### Example 6

### 4″-O-{3-[2-(3-Carboxy-1-ethyl-4-oxo-1,4-dihydro-6-quinolinylsulfanyl)ethylamino]propionyl}-6-O-methyl-erythromycin A

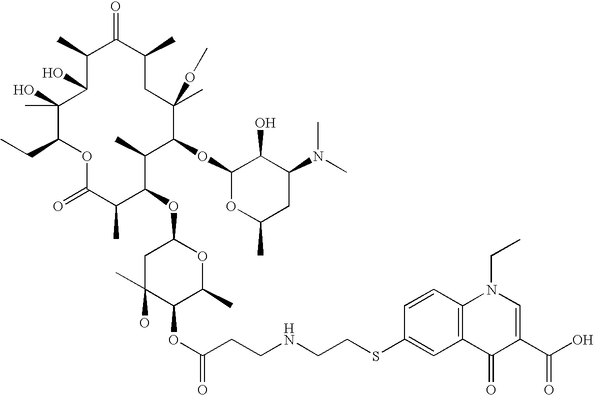

### a) 2′-O-Acetyl-4″-O-{3-[2-(3-carboxy-1-ethyl-4-oxo-1,4-dihydro-6-quinolinylsulfanyl)ethylamino]propionyl}-6-O-methyl-erythromycin A

Using the procedure of Example 3a, Intermediate 11 and Intermediate 6 (0.078 g, 0.16 mmol) gave the title compound, ESMS m/z 1135 [MH]⁻ (100%).

### b) 4″-O{3-[2-(3-Carboxy-1-ethyl-4-oxo-1,4-dihydro-6-quinolinylsulfanyl)ethylamino]propionyl}-6-O-methyl-erythromycin A

The compound obtained from Example 6a was dissolved in methanol (3 mL) and heated at 50° C. for 7 h, then allowed to stir at 25° C. overnight, followed by a further 5 h at 50° C. The mixture was evaporated to yield the desired compound as a white solid, ESMS m/z 1093 [M−H]⁻ (100%).

### Example 7

### 4″-O-{3-[2-(3-Carboxyl-1-ethyl-4-oxo-1,4-dihydro-6-quinolinylsulfanyl)ethylamino]propionyl}-6-O-methyl-erythromycin A methyl ester

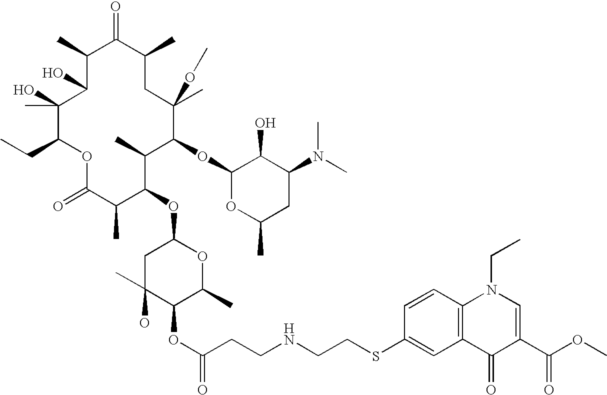

### a) 2′-O-Acetyl-4″-O-{3-[2-(3-carboxy-1-ethyl-4-oxo-1,4-dihydro-6-quinolinylsulfanyl)ethylamino]propionyl}-6-O-methyl-erythromycin A methyl ester

To a solution of Example 6a (0.078 g, 0.068 mmol) in methanol (1 mL) at room temperature was added a 0.5 M solution of trimethylsilyl diazomethane in hexanes (0.3 mL). After 2 h the reaction mixture was quenched with acetic acid (0.1 mL) and the solvent evaporated. The residue was chromatographed over silica gel eluting with dichloromethane containing an increasing concentration of methanol/ammonium hydroxide to yield the title compound, ESMS m/z 1150 [M+H]⁺ (80%).

### b) 4″-O-{3-[2-(3-Carboxy-1-ethyl-4-oxo-1,4-dihydro-6-quinolinylsulfanyl)ethylamino]propionyl}-6-O-methyl-erythromycin A methyl ester

A solution of Example 7a (0.013 g, 0.011 mmol) in methanol (1 mL) was heated at 50° C. After 12 h the mixture was cooled and the solvent evaporated to yield the title compound as a white solid, ESMS m/z 1108 [M+H]⁺ (100%).

### Example 8

### 4″-O-{3-[2-(3-Carboxy-1-ethyl-4-oxo-1,4-dihydro-6-quinolinylsulfanyl)ethylamino]propionyl}-6-O-methyl-erythromycin A isopropyl ester

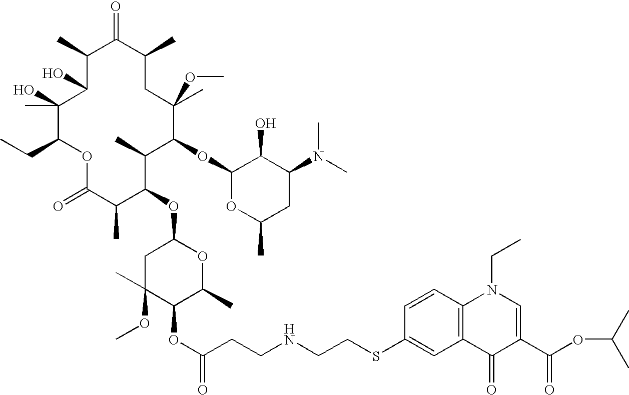

### a) 2′-O-Acetyl-4″-O-{3-[2-(3-carboxy-1-ethyl-4-oxo-1,4-dihydro-6-quinolinylsulfanyl)ethylamino]propionyl}-6-O-methyl-erythromycin A isopropyl ester

Using the procedure described in Example 3a, Intermediate 11 (0.21 g, 0.25 mmol) and Intermediate 7 (0.084 g, 0.25 mmol) gave the title compound, ESMS m/z 1179 [M+H]⁺ (100%).

### b) 4″-O-{3-[2-(3-Carboxy-1-ethyl-4-oxo-1,4-dihydro-6-quinolinylsulfanyl)ethylamino]propionyl}-6-O-methyl-erythromycin A isopropyl ester

The compound (0.13 g, 0.11 mmol) obtained from Example 8a was dissolved in methanol (10 mL) and heated at 50° C. for 7 h, then allowed to stir at 25° C. overnight, followed by a further 7 h at 50° C. The mixture was evaporated to yield the desired compound as a yellow powder, ESMS m/z 1137 [M+H]⁺ (100%).

### Example 9

### 4″-O-{3-[2-(3-Carboxy-1-ethyl-4-oxo-1,4-dihydro-quinolinylsulfanyl)ethylamino]propionyl}-6-O-methyl-11-desoxy-11-(R)amino-erythromycin A11,12-carbamate bis trifluoroacetate salt

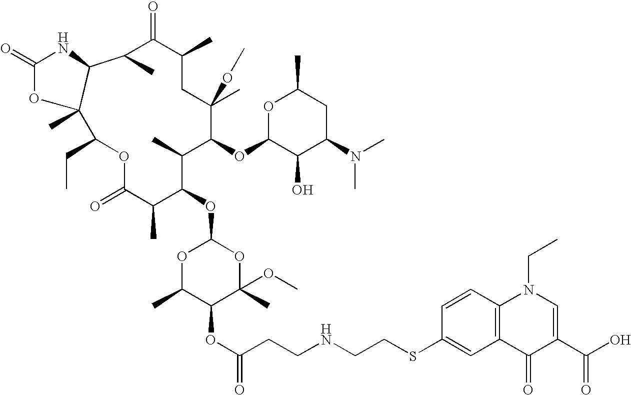

### a) 2′-O-Acetyl-4″-O-{3-[2-(3-carboxyl-1-ethyl-4-oxo-1,4-dihydro-6-quinolinylsulfanyl)ethylamino]propionyl}-6-O-methyl-11-desoxy-11(R)-amino-erythromycin A11,12-carbamate bis trifluoroacetate salt

Intermediate 14 (0.063 g, 0.072 mmol) and Intermediate 6 (0.044 g, 108 mmol) were mixed in DMSO (3 mL) containing water (5 drops) and triethylamine (0.06 mL) and heated under argon with stirring at 80° C. for 96 h. After removal of two thirds of the solvent by evaporation under reduced pressure methanol (1 mL) was added and the solution injected onto a preparative reverse phase HPLC to give the title compound (0.053 g, 53%); ESMS m/z 1161 [M+H]⁺.

### b) 4″-O-{3-[2-(3-Carboxy-1-ethyl-4-oxo-1,4-dihydro-4-quinolinylsulfanyl)ethylamino]propionyl})-6-O-methyl-11-desoxy-11-(R)-amino-erythromycin A11,12-carbamate bis trifluoroacetate salt

Example 9a (0.053 g) was dissolved in methanol (30 mL) and left at 20° C. for 70 h, 40° C. for 24 h, 50° C. for 17 h, 60° C. for 25 h and 70° C. for 96 h. After removal of the solvent by evaporation under reduced pressure the residue was purified by preparative reverse phase HPLC to give the title product as a yellow solid (0.028 g, 55%); ESMS m/z 1119 [M+H]⁺.

### Example 10

### 4″-O-{3-[2-(3-Carboxy-1-ethyl-4-oxo-1,4-dihydro-6-quinolinysulfanyl)ethylamino]propionyl}-azithromycin

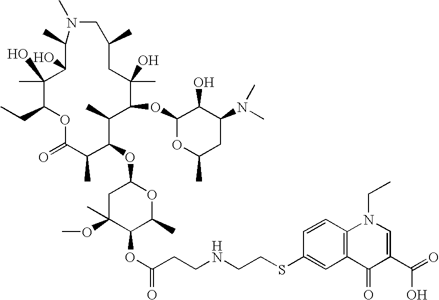

Using the procedure described in Example 2, Intermediate 10 (0.07 g, 0.087 mmol) and Intermediate 6 (0.14 g, 0.22 mmol) gave, after chromatography, the title compound, ESMS m/z 1093 [M−H]⁻ (50%).

### Example 11

### 4″-O-{3-[2-(3-Carboxy-1-ethyl-4-oxo-1,4-dihydro-6-quinolinysulfanyl)ethylamino]propionyl}-azithromycin11,12-carbonate

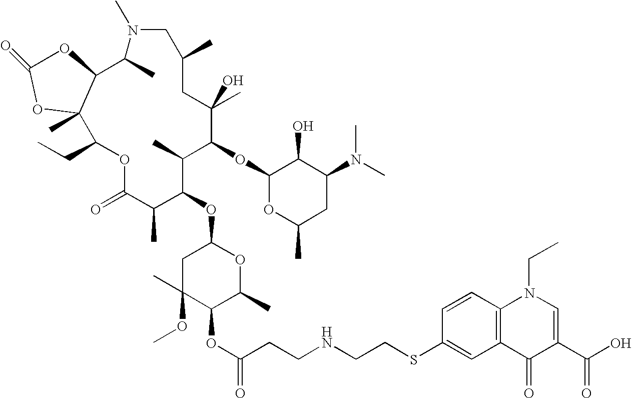

Using the procedure described in Example 2, Intermediate 9 (0.08 g, 0.1 mmol) and Intermediate 6 (0.107 g, 0.16 mmol) gave, after chromatography, the title compound, ESMS m/z 1119 [M−H]⁻ (100%).

### Example 12

### 4″-O-{3-[2-(6-Carboxy-7-oxo-2,3-dihydro-1H,7H-pyrido[3,2,1-ij]quinolin-9-yloxy)ethylamino]propionyl}-azithromycin tris trifluoroacetate salt

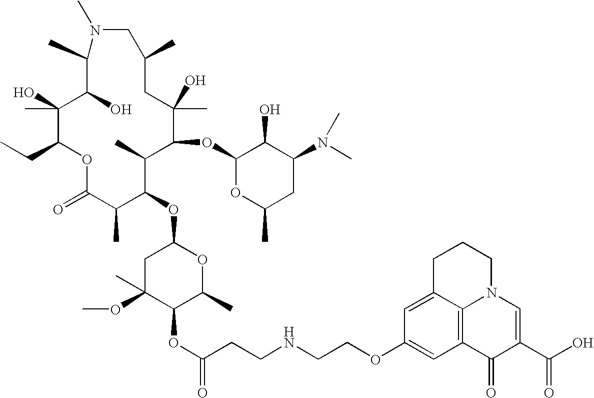

Intermediate 3 (0.049 g, 0.12 mmol) and Intermediate 10 (0.096 g, 0.12 mmol) were mixed in DMSO (0.5 mL) containing water (1 drop) and triethylamine (0.034 mL. 0.24 mmol). The container was flushed with argon and heated with stirring at 80° C. for 16 h. The reaction mixture was diluted with methanol and injected onto a preparative reverse phase HPLC. The product was obtained as an off-white solid (0.076 g, 44%); ESMS m/z 1091 [M+H]⁺ (10%), 546 [M+2H]²⁺ (100%).

### Example 13

### 4′-O-{3-[2-(6-Carboxy-7-oxo-2,3-dihydro-1H,7H-pyrido[3,2,1-ij]quinolin-9-yloxy)ethylamino]propionyl}-6-O-methyl-erythromycin A bis trifluoroacetate salt

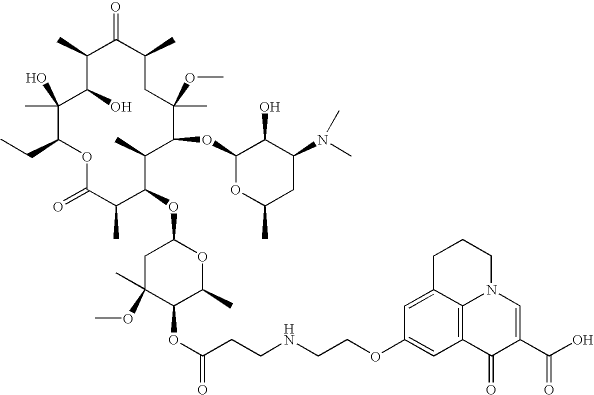

### a) 2′-O-Acetyl-4″-O-{3-[2-(6-carboxy-7-oxo-2,3-dihydro-1H,7H-pyrido[3,2,1-ij]quinolin-9-yloxy)ethylamino]propionyl}-6-O-methyl-erythromycin A

Intermediate 3 (0.049 g, 0.12 mmol) and Intermediate 11 (0.101 g, 0.12 mmol) were mixed in DMSO (0.5 mL) containing water (1 drop) and triethylamine (0.034 mL, 0.24 mmol). The container was flushed with argon and heated with stirring at 80° C. for 46 h. The reaction mixture was diluted with methanol and injected onto a preparative reverse phase HPLC. The impure product was obtained as a yellow solid (0.070 g). This material was further purified by chromatography on silica gel (1 g), eluting with 0-24% methanolic ammonia (2M) in dichloromethane, to give the title compound as a white solid (0.057 g, 42%); ESMS m/z 1132 [M+H]⁺ (100%).

### b) 4″-O-{3-[2-(6-Carboxy-7-oxo-2,3-dihydro-1H,7H-pyrido[3,2,1-ij]quinolin-9-yloxy)ethylamino]propionyl}-6-O-methyl-erythromycin A bis trifluoroacetate salt

Example 13a (0.057 g, 0.05 mmol) in methanol (4 mL) was heated under argon at 60° C. for 2 h, 30° C. for 15 h, 60° C. for 10 h, and 40° C. for 15 h. The solution was then evaporated to dryness and the crude product purified by column chromatography on silica (0.8 g), eluting with 030% methanolic ammonia (2M) in dichloromethane, to give the impure product as a white solid (0.048 g). This was further purified by preparative reverse phase HPLC to give the title compound as a white solid (0.023 g, 35%); ESMS m/z 1090 [M+H]⁺ (100%).

### Example 14

### 4″-O-{3-[2-(3-Carboxy-1-ethyl-4-oxo-1,4-dihydro-6-quinolinyloxy)ethylamino]propionyl}-azithromycin tris trifluoroacetate salt

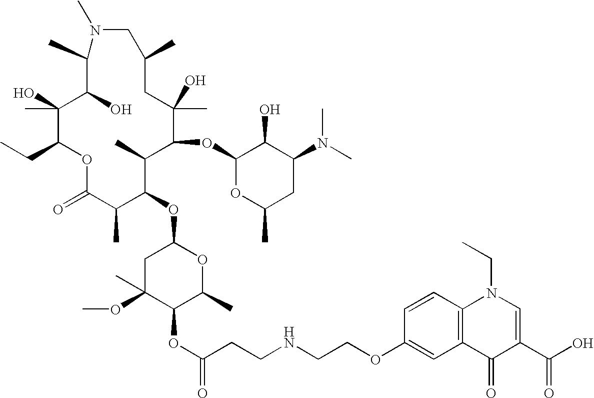

Intermediate 4 (0.045 g, 0.12 mmol) and Intermediate 10 (0.096 g, 0.12 mmol) were mixed in DMSO (0.5 mL) containing water (1 drop) and triethylamine (0.034 mL). The container was flushed with argon and heated with stirring at 80° C. for 22 h. The reaction mixture was diluted with methanol and injected onto a preparative reverse phase HPLC. The product was obtained as an off-white powder (0.116 g, 68%); ESMS m/z 1079 [M+H]⁺ (30%), 540 [M+2H]²⁺ (100%).

### Example 15

### 4″-O-{3-[2-(3-Carboxy-1-ethyl-4-oxo-1,4-dihydro-6-quinolinyloxy)ethylamino]propionyl}-6-O-methyl-erythromycin A monoformate salt

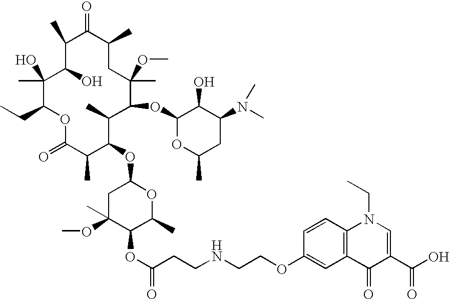

Intermediate 4 (0.045 g, 0.12 mmol) and Intermediate 12 (0.096 g, 0.12 mmol) were mixed in DMSO (0.5 mL) containing water (1 drop) and triethylamine (0.034 mL). The container was flushed with argon and heated with stirring at 80° C. for 27 h. The reaction mixture was diluted with methanol and injected onto a preparative reverse phase HPLC. The impure product was obtained as an off-white foam (0.101 g). This was further purified by preparative HPLC (MDAP) to give the product as an off-white powder (0.024 g, 18%); ESMS m/z 1078 [M+H]⁺ (100%).

### Example 16

### 4″-O-{3-[2-(3-Carboxy-1-ethyl-4-oxo-1,4-dihydro-7-quinolinyloxy)ethylamino]propionyl}azithromycin

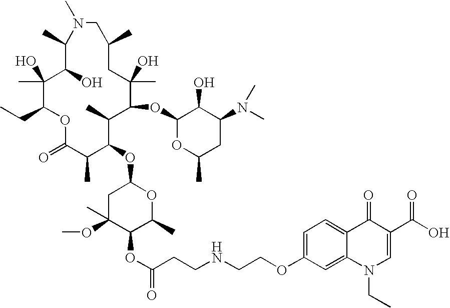

Intermediate 5 (0.030 g, 0.1 mmol) and Intermediate 10 (0.08 g, 0.1 mmol) were mixed in DMSO (0.5 mL) containing water (1 drop) and triethylamine (0.014 mL, 0.1 mmol). The container was flushed with argon and heated with stirring at 80° C. for 65 h. The reaction mixture was diluted with methanol and injected onto a preparative reverse phase HPLC. The impure product was obtained as an off-white powder (0.039 g). This material was further purified by column chromatography on silica gel (0.6 g), eluting with 5-25% methanolic ammonia (2M) in dichloromethane, to give the pure product as a white solid (0.014 g, 13%); ESMS m/z 1079 [M+H]⁺ (10%), 540 [M+2H]²⁺ (100%).

### Example 17

### 4″-O-{3-[2-(3-Carboxy-1-ethyl-4-oxo-1,4-dihydro-7-quinolinyloxy)ethylamino]propionyl}-6-O-methyl-erythromycin A

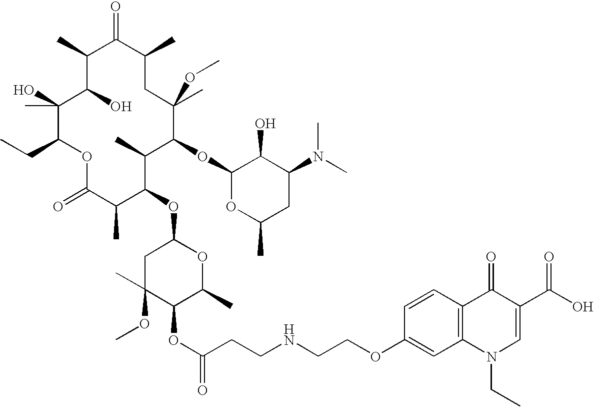

Using the procedure described in Example 3, Intermediate 11 (0.1 g, 0.12 mmol) and Intermediate 5 (0.036 g, 0.12 mmol) gave the title compound, ¹H NMR 8 (CD₃OD) inter alia 5.14 (1H, d, J=8.8 Hz), 6.96 (1H, d, J=1.7 Hz), 7.15 (1H, dd, J=9.4 & 2.0 Hz), 8.47 (1H, d, J=9.0 Hz), 8.71 (1H, s).

### Example 18

### 4″-O-{3-[2-(3-Carboxy-7-chloro-1-cyclopropyl-4-oxo-1,4-dihydro-4-quinolinylamino)ethoxy]propionyl}-azithromycin

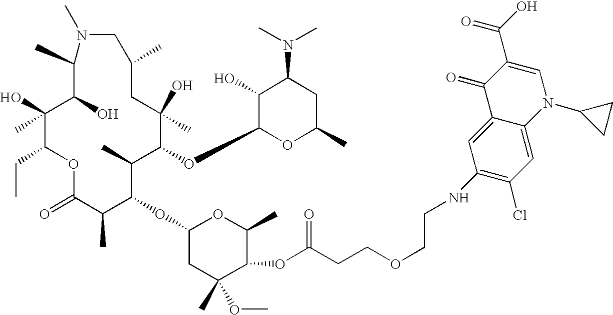

a) To a solution of Intermediate 17 (0.9 g, 2.28 mmol) in DMF (12 mL), a solution of 2′-1-acetyl-azithromycin (0.5 g, 0.63 mmol) in DCM (12 mL) was added and the mixture was cooled to 0° C. To the reaction mixture EDC.HCl (0.6 g, 3.13 mmol) and DMAP (0.16 g, 1.31 mmol) was added and the reaction mixture was stirred at 0° C. to room temperature for 20 hours. Additional amounts of EDC.HCl (0.6 g, 3.13 mmol) and DMAP (0.16 g, 1.31 mmol) were added and the reaction mixture were stirred at room temperature for an additional 4 hours. The DCM was evaporated. To the residue, water and EtOAc were added and the layers were separated. The water layer was extracted twice with EtOAc. The combined organic layers were dried over K₂CO₃ and evaporated yielding crude 2′-protected product in a mixture with starting compounds (incomplete conversion). The product obtained was dissolved in MeOH (100 mL) and the solution was stirred for 16 hours at room temperature and for 8 hours at 60° C. The methanol was evaporated under reduced pressure and the residue was dissolved in DCM and washed with brine (5×). Evaporation of organic layer yielded product which was precipitated twice from EtOAc:n-hexane yielding the title compound (0.17 g).

b) To a solution of Intermediate 17 (0.50 g, 1.266 mmol) in DCM (10 mL), TEA (0.312 mL, 2.24 mmol) was added and the mixture was cooled to 0° C. under N₂ atmosphere. To this mixture, pivaloyl chloride (0.276 mL, 2.24 mmol) was added and the mixture was stirred for 1 hour at 0° C. 2′-O-acetyl-azithromycin (0.5 g, 0.633 mmol) and DMAP (0.464 g, 3.80 mmol) were then added and the reaction mixture was stirred at 0° C. to room temperature for 16 hours. Water was added to the reaction mixture and the layers were separated. The water layer was extracted with DCM (2×) and the combined organic layers were washed with brine, dried over K₂CO₃ and then evaporated under reduced pressure. Methanol (70 mL) was added to the residue and the reaction mixture was stirred for 18 hours at 65° C. Evaporation of methanol yielded 660 mg of crude Example 18 which was precipitated twice from EtOAc/n-hexane yielding 400 mg of pure title product.

¹H-NMR (500 MHz, DMSO-d6) δ: 8.73 (s, 1H), 8.06 (s, 1H), 7.53 (s, 1H), 5.20 (d, 1H), 5.03 (t, 1NH), 4.72 (d, 1H), 4.70 (t, 1H), 4.56 (d, 1H), 4.41 (m, 1H), 4.25 (dd, 1H), 3.80 (m, 2H), 3.79 (m, 1H), 3.78 (m, 2H), 3.68 (d, 1H), 3.59 (m, 1H), 3.58 (m, 1H), 3.47 (m, 2H), 3.31 (s, 3H), 3.29 (m, 1H), 2.75 (m, 1H), 2.68 (m, 2H), 2.65 (m, 1H), 2.55 (d, 1H), 2.40 (m, 1H), 2.39 (s, 6H), 2.32 (m, 1H), 2.31 (s, 3H), 2.07 (m, 1H), 1.99 (m, 1H), 1.90 (m, 1H), 1.75 (d, 1H), 1.64 (dd, 1H), 1.46 (m, 1H), 1.40 (m, 2H), 1.27 (s, 3H), 1.25 (m, 1H), 1.21 (m, 2H), 1.20 (d, 3H), 1.19 (m, 3H), 1.13 (d, 3H), 1.11 (s, 3H), 1.10 (m, 3H), 1.09 (s, 3H), 1.04 (d, 3H), 0.91 (m, 3H), 0.90 (t, 3H); ¹³C-NMR (125 MHz, DMSO-d6) δ: 178.87, 177.55, 171.18, 167.32, 145.93, 142.95, 132.72, 127.65, 126.28, 118.14, 107.66, 104.52, 102.19, 94.68, 83.22, 79.02, 77.72, 77.49, 74.28, 73.67, 73.61, 72.96, 70.97, 70.06, 68.75, 67.74, 66.32, 65.61, 62.98, 62.54, 49.48, 45.17, 43.30, 42.22, 42.08, 40.43, 36.30, 35.38, 35.99, 27.50, 26.78, 21.99, 21.79, 21.31, 21.24, 17.77, 16.21, 14.59, 11.29, 9.15, 8.14, 8.10, 7.45; MS; m/z (ES): 1125.40 [MH]⁺.

### Example 19

### 4″-O-{3-[2-(3-Carboxy-1-cyclopropyl-4-oxo-1,4-dihydro-6-quinolinylamino)ethoxy]propionyl}-azithromycin

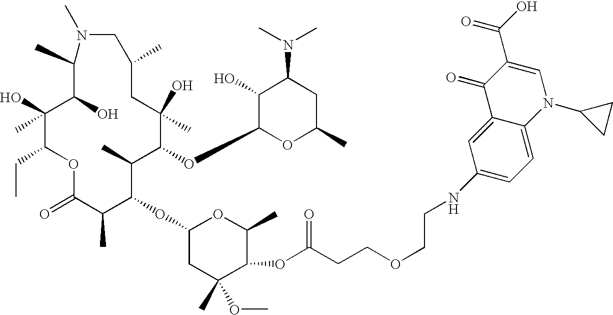

To solution of Example 18 (2 g) in MeOH (50 mL), 10% Pd/C (1 g) was added and the reaction mixture was shaken in Parr apparatus at 5 bar for 21 hours. The catalyst was filtered off and the solvent evaporated under reduced pressure. DCM and water were added to the residue and the pH value was adjusted to 9.5. The layers were separated and the water layer extracted with DCM (2×). Combined organic layers were washed with brine, dried over K₂CO₃ and evaporated yielding 1.35 g of crude title product. After purification on column using eluent DCM:MeOH:NH₃=90:3:0.3, of pure title compound (800 mg); ¹H-NMR (500 MHz, DMSO-d6) δ: 8.72 (s, 1H), 7.92 (d, 1H), 7.46 (d, 1H), 7.17 (dd, 1H), 5.17 (d, 1H), 4.75 (d, 1H), 4.73 (m, 1H), 4.56 (d, 1H), 4.24 (m, 1H), 4.27 (dd, 1H), 3.80 (m, 2H), 3.79 (m, 1H), 3.75 (t, 2H), 3.68 (d, 1H), 3.61 (m, 1H), 3.60 (m, 11H), 3.39 (q, 2H), 3.32 (s, 3H), 3.26 (m, 1H), 2.78 (m, 1H), 2.70 (m, 11H), 2.64 (m, 2H), 2.60 (m, 1H), 2.52 (m, 1H), 2.41 (d, 1H), 2.36 (s, 6H), 2.32 (s, 3H), 2.00 (m, 1H+1H), 1.90 (m, 1H), 1.76 (d, 1H), 1.65 (dd, 1H), 1.48 (m, 1H), 1.37 (m, 2H), 1.27 (s, 3H), 1.21 (m, 3H), 1.20 (m, 3H), 1.19 (m, 2H), 1.16 (d, 3H), 1.12 (s, 3H), 1.10 (m, 3H), 1.09 (s, 3H), 1.05 (d, 3H), 0.90 (m, 3H), 0.89 (t, 3H); ¹³C-NMR (125 MHz, DMSO-d6) δ: 178.77, 177.93, 171.67, 167.73, 145.93, 145.17, 133.40, 127.62, 122.54, 118.44, 107.43, 104.05, 102.22, 94.85, 83.28, 79.17, 77.99, 77.52, 74.33, 74.04, 73.59, 72.93, 71.00, 70.07, 68.89, 67.78, 66.16, 65.56, 62.95, 62.37, 49.50, 45.08, 43.24, 42.26, 41.72, 40.39, 36.41, 35.40, 35.09, 35.00, 27.43, 26.77, 21.98, 21.80, 21.26, 21.24, 17.90, 16.20, 14.82, 11.27, 9.24, 8.10, 8.08, 7.58; MS; m/z (ES): 1092.15 [MH]⁺

### Example 20

### 4″-O-{3-[2-(3-Carboxy-7-chloro-1-cyclopropyl-4-oxo-1,4-dihydro-6-quinolinylamino)ethoxy]propionyl}azithromycin11,12-cyclic carbonate

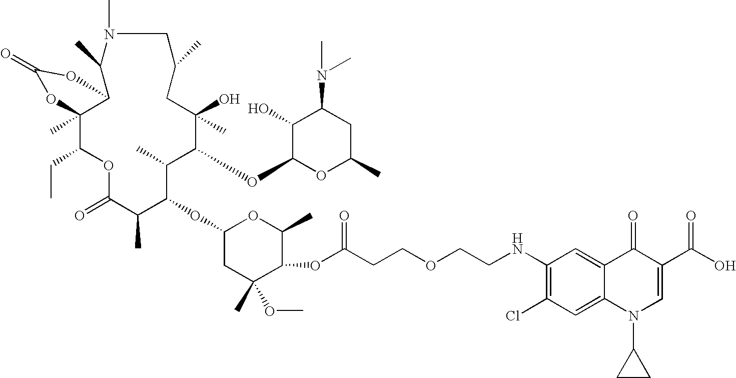

A DCM (10 mL) and TEA (0.210 mL, 1.51 mmol) solution of Intermediate 17 (0.64 g, 1.62 mmol) was cooled to 0° C. To this mixture, EDC.HCl (0.6 g, 3.13 mmol) was added and the reaction mixture was stirred at 0° C. to room temperature for 1 hour. 2′-O-Acetyl-azithromycin 11,12-carbonate (0.62 g, 0.76 mmol) and DMAP (0.185 g, 1.51 mmol) were then added and the reaction mixture was stirred at 0° C. to room temperature for 23 hours. Because the reaction was not completed additional amounts of EDC.HCl (0.6 g, 3.13 mmol), DMAP (0.185 g, 1.51 mmol) and TEA (0.210 mL, 1.51 mmol) were added and the mixture stirred for 19 hours at room temperature. Water and EtOAc were added and the layers were separated. The water layer was extracted twice with EtOAc. The combined organic layers were dried over K₂CO₃ and evaporated. The product obtained was dissolved in MeOH (70 mL) and the solution was stirred for 21 hours at 60° C. The methanol was evaporated under reduced pressure and the residue was precipitated from EtOAc:n-hexane yielding 0.39 g of crude product which was purified by column chromatography (DCM:MeOH:NH₃=90:4:0.5) yielding two products. Both products were precipitated form EtOAc:n-hexane yielding 60 mg of pure title compound; ¹H NMR (500 MHz, DMSO-d6) δ: 8.73 (s, 1H), 8.05 (d, 1H), 7.53 (d, 1H), 5.09 (d 1H), 5.03 (t, 1H), 4.86 (dd, 1H), 4.73 (d, 1H), 4.49 (d, 1H), 4.39 (m, 1H), 4.37 (m, 1H), 4.36 (m, 1H), 3.84 (m, 2H), 3.80 (m, 1H), 3.78 (m, 2H), 3.58 (m, 1H), 3.56 (d, 1H), 3.46 (q, 2H), 3.32 (m, 1H), 3.30 (s, 3H), 2.86 (m, 1H), 2.85 (m, 1H), 2.77 (m, 1H), 2.66 (m, 2H), 2.47 (s, 6H), 2.43 (m, 1H), 2.36 (d, 1H), 2.20 (s, 3H), 2.04 (m, 1H), 1.99 (m, 1H), 1.90 (m, 1H), 1.82 (m, 1H), 1.64 (dd, 1H), 1.55 (m, 1H), 1.44 (s, 3H), 1.40 (m, 1H), 1.31 (m, 1H), 1.24 (s, 3H), 1.22 (m, 2H), 1.20 (d, 3H), 1.19 (d, 3H), 1.12 (d, 3H), 1.11 (s, 3H), 1.06 (d, 3H), 1.02 (d, 3H), 0.91 (t, 3H), 0.90 (m, 3H); ¹³C-NMR (75 MHz, DMSO-d6) δ: 177.56, 177.20, 171.25, 167.40, 153.34, 145.92, 142.98, 132.71, 127.68, 126.24, 118.18, 107.60, 104.48, 102.49, 95.23, 85.95, 85.07, 78.93, 77.79, 76.44, 73.33, 73.08, 70.74, 68.72, 68.03, 67.58, 66.35, 65.41, 62.98, 61.30, 49.59, 45.21, 43.29, 43.11, 41.92, 40.51, 35.41, 35.19, 35.02, 34.35, 30.00, 26.81, 26.25, 22.15, 22.03, 21.49, 21.20, 17.68, 14.77, 14.15, 10.90, 10.46, 8.11, 5.54; MS; m/z (ES): 1150.7 [MH]⁺.

### Example 21

### 4″-O-{3-[2-(3-Carboxy-1-cyclopropyl-4-oxo-1,4-dihydro-6-quinolinylamino)ethoxy]propionyl}-azithromycin11,12-cyclic carbonate

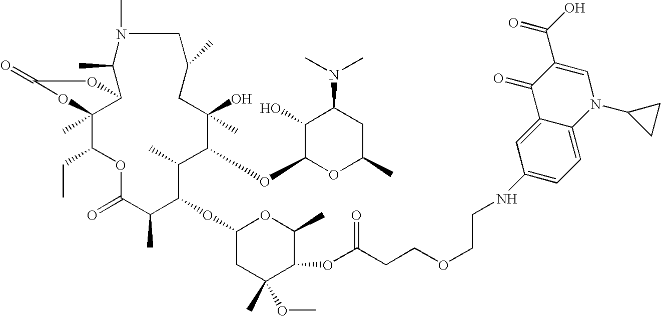

To a solution of Example 20 (0.044 g) in MeOH (10 mL), 10% Pd/C (0.020 g) was added and the reaction mixture was shaken at 5 bar for 22 hours. The catalyst was filtered off and the solvent evaporated under reduced pressure. DCM and water were added to the residue and the pH value was adjusted to 9.5. The layers were separated and the water layer extracted with DCM (2×). The combined organic layers were washed with brine, dried over K₂CO₃ and evaporated yielding 30 mg of crude title product. Precipitation from EtOAc/n-hexane yielded pure title compound (22 mg); MS; m/z (ES): 1117.5 [MH]⁺.

### Example 22

### 4″-O-{3-[2-(3-(2,2-Dimethyl-proplonyloxymethoxycarbonyl)-1-cyclopropyl-4-oxo-1,4-dihydro-6-quinolinylamino]ethoxy}propionyl)-azithromycin

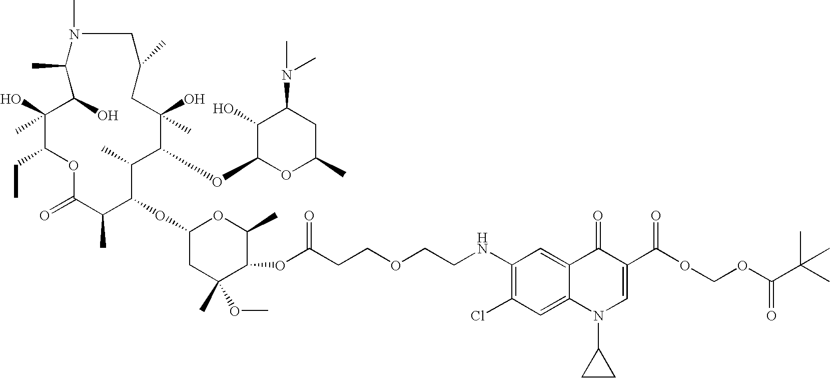

To a solution of Example 18 (0.09 g, 0.08 mmol) in DMF (10 mL), K₂CO₃ (0.0137 g, 0.0993 mmol) was added and the mixture was stirred at room temperature for 1 hour. Pivaloyloxymethyl chloride (0.015 mL, 0.104 mmol) was added to the reaction mixture and the mixture was stirred at room temperature for 23 hour. The reaction was not complete so additional amount of pivaloyloxymethyl chloride (0.005 mL, 0.035 mmol) was added. The reaction mixture was stirred for additional 24 hours at room temperature but conversion was again not complete. Thus, K₂CO₃ (0.011 g, 0.0797 mmol) was added and after 1 hour pivaloylmethylchloride (0.01 mL, 0.07 mmol) was added. The reaction mixture was stirred for an additional 24 hours at room temperature and then extracted with EtOAc. The combined organic layer washed with brine, dried over anhydrous K₂CO₃, filtered and evaporated under reduced pressure yielding 100 mg of crude product. Purification by column chromatography (SPE-column, gradient polarity: 100% DCM to DCM:MeOH:NH₃=90:9:0.5) yielded 80 mg of product which was precipitated from EtOAc:n-hexane yielding 49 mg of pure title product; MS; m/z (ES): 1240.20 [MH]⁺.

### Example 23

### 4″-O-{3-[2-(3-Carboxy-7-chloro-1-cyclopropyl-4-oxo-1,4-dihydro-6-quinolinylamino)ethoxy]propionyl}-11-O-methyl-azithromycin

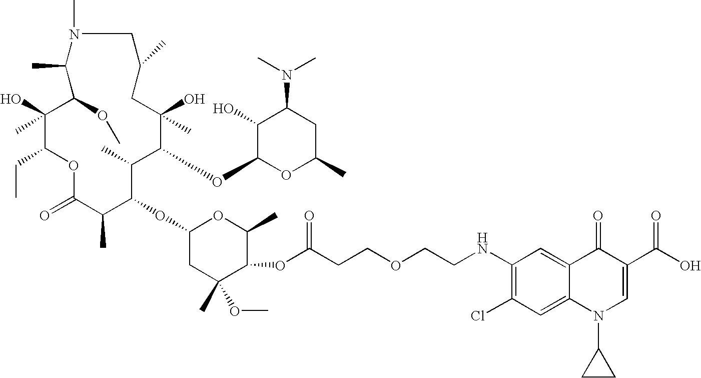

To a DCM (60 mL) solution of Intermediate 17 (0.98 g, 2,484 mmol), TEA (0.5 mL, 4,968 mmol) was added and the mixture was cooled to 0° C. under N₂ atmosphere. Pivaloyl chloride (0.6 mL, 4968 mmol) was added to this mixture and the mixture was stirred for 1 hour at 0° C. Intermediate 22 (1.0 g, 1.242 mmol) and DMAP (0.91 g, 7.452 mmol) were then added and the reaction mixture was stirred at 0° C. to room temperature for 48 hours. The reaction mixture was cooled to 0° C., Intermediate 17 (0.49 g), TEA (0.35 mL) and pivaloyl chloride (0.31 mL) were added and the reaction mixture was stirred at 0° C. to room temperature for 48 hours. Water was added to the reaction mixture and the layers were separated. The water layer was extracted with DCM (2×) and the combined organic layers were washed with brine, dried over K₂CO₃ and evaporated under reduced pressure To the residue methanol (70 mL) was added and the reaction mixture was stirred for 18 hours at 60° C. Evaporation of methanol gave crude product which was precipitated twice from EtOAc/n-hexane yielding 0.358 g of pure title product; MS: m/z (ES): 1140.8 [MH]⁺.

### Example 24

### 4″-O-{3-[2-(3-Carboxy-1-cyclopropyl-4-oxo-1,4-dihydro-6-quinolinylamino)ethoxy]propionyl}-11-O-methyl-azithromycin

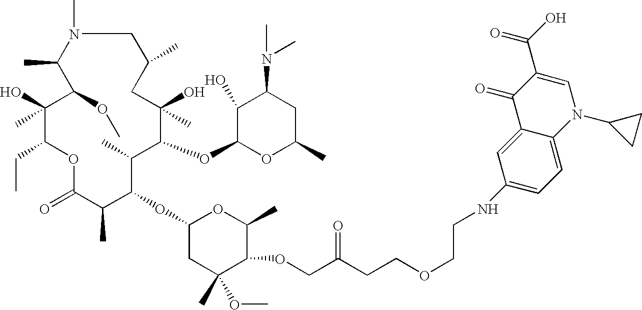

To a MeOH (50 mL) solution of Example 23 (1.1 g), 10% Pd/C (0.5 g) was added and the reaction mixture was shaken at 5 bar for 24 hours. The catalyst was filtered off and the solvent evaporated under reduced pressure. DCM and water were added to the residue and the pH value was adjusted to 9.5. The layers were separated and the water layer extracted with DCM (2×). The combined organic layers were washed with brine, dried over K₂CO₃ and evaporated yielding 0.861 g of title product; MS: m/z (ES): 1106.4 [MH]⁺.

### Example 25

### 4″-O-{3-[2-(3-Carboxy-7-chloro-1-cyclopropyl-4-oxo-1,4-dihydro-quinolin-6-yloxy)-ethoxy]-propionyl}-azithromycin11,12-carbonate

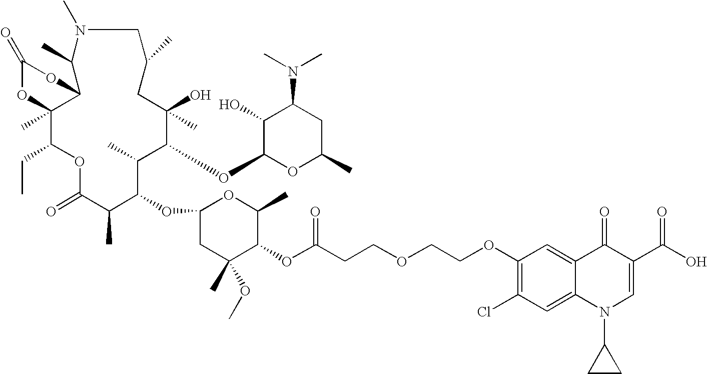

To a solution of Intermediate 20 (0.18 g) in DMC (10 mL), TEA (0.06 mL) and 2′-O-acetyl-azithromycin11,12-carbonate (0.372 g) were added and the mixture was cooled to 0° C. EDC.HCl (0.175 g) and DMAP (0.056 g) were added to this mixture and the reaction mixture was stirred at room temperature for 48 h. Additional EDC.HCl (0.175 g) in DMF (10 mL) was then added and the mixture stirred at 40° C. for a further 48 hours. Water and EtOAc were added to the reaction solution and the layers were separated. The water layer was extracted twice with EtOAc. The combined organic layers were dried over K₂CO₃ and evaporated yielding crude 2′-protected product, which was dissolved in MeOH and the solution was stirred for 24 hours at room temperature. Purification by column chromatography (DCM:MeOH:NH₃=90:9:1.5) yielded 0.05 g of the title product; MS m/z 1151.18 (MH+).

### Example 26

### 4″-O-{3-[2-(3-Carboxy-1-cyclopropyl-4-oxo-1,4-dihydro-quinolin-6-yloxy)-ethoxy]-propionyl}-azithromycin11,12-carbonate

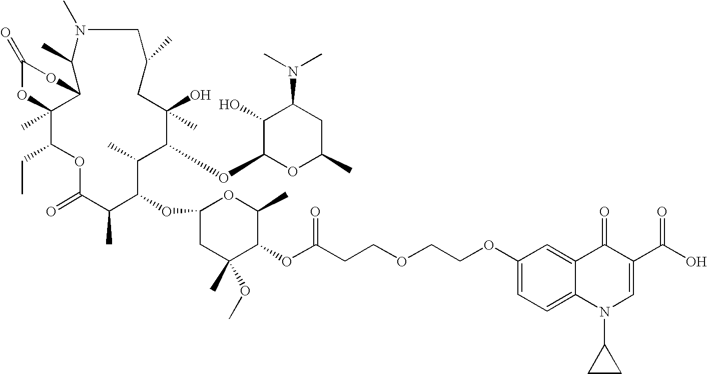

To Example 25 (25 mg), methanol (10 mL) and 10% Pd/C (20 mg) were added. Hydrogenolysis was performed at 4×10⁵ Pa for 4 h. The reaction mixture was filtered and the filtrate evaporated. Purification by column chromatography (DCM:MeOH:NH₃=90:1.5:1.5) yielded 0.01 g of the title product; MS m/z=1118.55 (MH+).

### Example 27

### 4″-O-{3-[2-(3-Carboxy-7-chloro-1-cyclopropyl-4-oxo-1,4-dihydro-quinolin-6-yloxy)ethoxy]-propionyl}-azithromycin

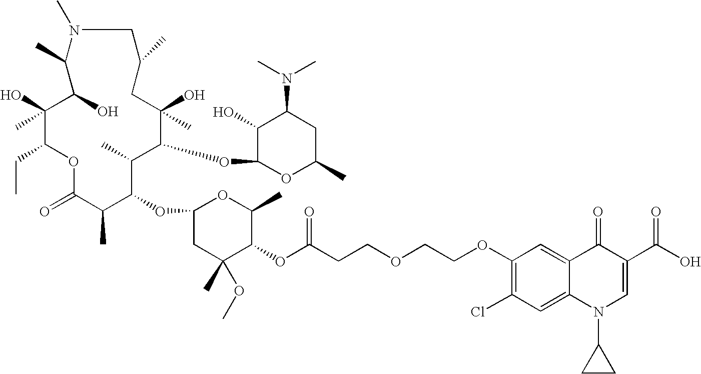

A solution of Intermediate 20 (0.50 g) in DCM (10 mL), was cooled to 0° C. under argon atmosphere. EDC.HCl (0.36 g)) was added to the solution followed by 2′-O-acetyl-azithromycin and DMAP (0.150 g). The reaction mixture was stirred at 0° C. to room temperature for 30 h. Water and EtOAc (50 mL) were added to the reaction solution and the layers were separated. The water layer was extracted twice with EtOAc (30 mL). The combined organic layers were dried over K₂CO₃ and evaporated yielding crude 2′-protected product which was dissolved in MeOH and the solution stirred for 24 hours at 50° C. Purification by column chromatography (DCM:MeOH:NH3=90:9:0.5) yielded title product which was precipitated from EtOAc:n-hexane yielding the title product (0.35 g) as a white solid; ¹³C-NMR (75 MHz, DMSO-d6) δ: 178.7, 177.5, 171.3, 166.8, 153.1, 147.3, 135.6, 131.3, 125.8, 119.2, 108.4, 107.6, 102.0, 94.7, 83.3, 78.9, 77.7, 74.3, 73.6, 72.9, 70.9, 70.0, 69.4, 69.1, 67.6, 67.1, 63.0, 49.4, 45.1, 41.9, 36.3, 35.5, 35.2, 35.0, 27.4, 26.7, 22.0, 21.7, 21.3, 21.2, 17.7, 16.2, 14.7, 11.3, 9.2, 8.3, 7.5; MS; m/z (ES): [MH]⁺.

### Example 28

### 4″-O-(3-{2-[7-chloro-1-cyclopropyl-3-(2,2-dimethyl-propionyloxymethoxycarbonyl)-4-oxo-1,4-dihydro-quinolin-6-yloxy]-ethoxy}-propionyl)-azithromycin

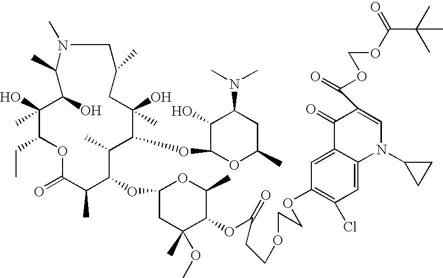

To a solution of Example 27 (0.125 g) in DMF (6 mL) at room temperature, K₂CO₃ (18 mg) was added and the mixture was stirred for 2 h. Chloromethyl pivalate (25 μL) was added to the reaction mixture and the mixture was stirred at 35° C. for 24 h. EtOAc (30 mL) was added to the reaction solution which was then extracted with H₂O (3×10 mL). The organic layer was dried and evaporated. The residue was precipitated from EtOAc:n-hexane affording the title compound (105 mg); MS; m/z (ES): 1240.2 [MH]⁺.

### Example 29

### 4″-O-{3-[2-(3-Carboxy-1-cyclopropyl-4-oxo-1,4-dihydro-quinolin-6-yloxy)-ethoxy]-propionyl}-azithromycin

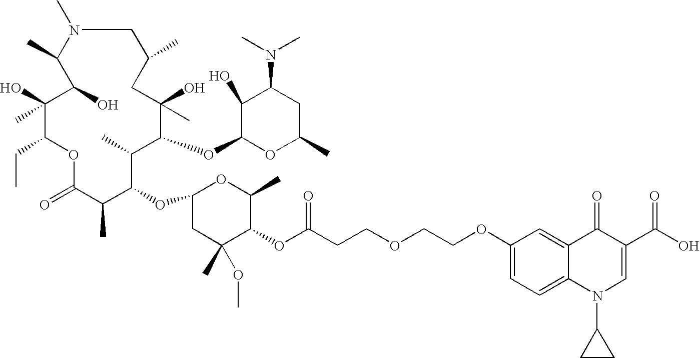

To a solution of Example 27 (1.03 g) in MeOH (30 mL), 10% Pd/C (0.5 g) was added and the reaction mixture was shaken at 4.6 bar overnight. The catalyst was filtered off and the solvent evaporated under reduced pressure. The residue was purified by column chromatography (SPE-column, DCM:MeOH:NH₃=90:9:0.5) yielding title product which was precipitated from Et-Ac/diisopropyl-ether to give 0.1 g of the title product; MS; m/z: 1094.04 [MH]⁺; ¹³C-NMR(75 Hz, CDCl₃) δ: 178.70, 177.96, 171.35, 167.22, 157.27, 146.67, 135.75, 127.54, 124.94, 118.99, 108.02, 106.52, 101.88, 94.72, 84.21, 78.89, 77.69, 74.26, 73.57, 72.99, 70.94, 69.93, 69.34, 68.34, 68.01, 67.55, 66.89, 65.59, 63.01, 62.70, 49.43, 45.15, 42.19, 41.96, 40.81, 36.34, 35.55, 35.20, 34.96, 27.37, 26.71, 22.86, 22.01, 21.66, 21.18, 16.23, 14.65, 11.27, 9.29, 8.27, 7.54.

### Example 30

### 4″-O-{3-[2-(3-Carboxy-7-chloro-1-cyclopropyl-4-oxo-1,4-dihydro-6-quinolinylamino)ethoxy]propionyl}-6-O-methyl-11-desoxy-11-(R)-amino-erythromycin A11,12-carbamate

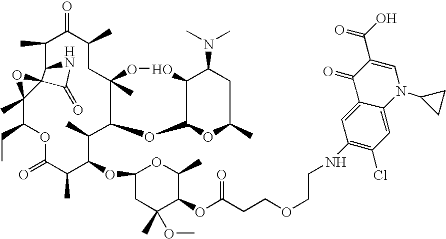

To a DCM (40 mL) solution of Intermediate 17 (0.727 g, 1.84 mmol), TEA (0.512 mL, 3.68 mmol) was added and the mixture was cooled to 0° C. under N₂ atmosphere. Pivaloyl chloride (0.453 mL, 3.68 mmol) was added to this mixture and the mixture was stirred for 1 hour at 0 “C. 2′-O-Acetyl-azithromycin (0.75 g, 0.920 mmol) and DMAP (0.665 g, 5.52 mmol) were then added and the reaction mixture was stirred at 0° C. to room temperature for 24 hours. Water was added to the reaction mixture and the layers were separated. The water layer was extracted with DCM (2×) and the combined organic layers were washed with brine, dried over K₂CO₃, and evaporated under reduced pressure. Methanol (70 mL) was added to the residue and the reaction mixture was stirred for 24 hours at 65° C. Evaporation of methanol yielded 1.052 g of crude product which was precipitated twice from EtOAc/n-hexane yielding 853 mg of pure title product; MS; m/z (ES): 1151.6 [MH]⁺.

### Example 31

### 4″-O-{3-[2-(3-Carboxy-1-cyclopropyl-4-oxo-1,4-dihydro-6-quinolinylamino)ethoxy]propionyl}-6-O-methyl-11-desoxy-11-(R)-amino-erythromycin A11,12-carbamate

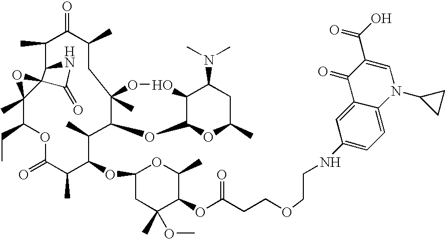

To a MeOH (25 mL) solution of Example 30 (500 mg), 10% Pd/C (50 mg) was added and the reaction mixture was shaken at 5 bar H₂ for 12 hours. The catalyst was filtered off and the solvent evaporated under reduced pressure. DCM and water were added and the pH value was adjusted to 9.5. The layers were separated and the water layer extracted with DCM (2×). The combined organic layers were washed with brine, dried over K₂CO₃ and evaporated yielding 356 g of pure title compound; MS; m/z (ES): 1114.9 [MH]⁺.

### Example 32

### 4″-O-{3-[2-(3-Carboxy-7-chloro-1-cyclopropyl-4-oxo-1,4-dihydro-quinolin-6-yloxy)ethoxy]propionyl}-6-O-methyl-11-desoxy-11-(R)-amino-erythromycin A11,12-carbamate

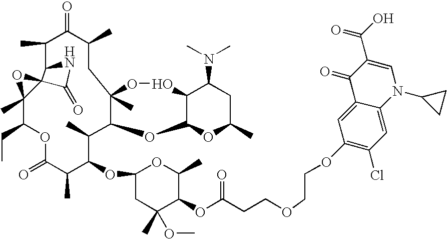

To a DCM (40 mL) solution of Intermediate 20 (0.728 g, 1.84 mmol), TEA (0.512 mL, 3.68 mmol) was added and the mixture was cooled to 0° C. under N₂ atmosphere. Pivaloyl chloride (0.453 mL, 3.68 mmol) was added to this mixture and the mixture was stirred for 1 hour at 0° C. 2′-O-Acetyl-azithromycin (0.75 g, 0.920 mmol) and DMAP (0.665 g, 5.52 mmol) were then added and the reaction mixture was stirred at 0° C. to room temperature for 48 hours. Water was added to the reaction mixture and the layers were separated. The water layer was extracted with DCM (2×) and the combined organic layers were washed with brine, dried over K₂CO₃, evaporated under reduced pressure. Methanol (70 mL) was added to the residue and the reaction mixture was stirred for 24 hours at 65° C. Evaporation of methanol yielded 1.030 g of crude product which was precipitated twice from EtOAc/n-hexane yielding 450 mg of product 85% pure. After purification on column using eluent DCM:MeOH:NH₃=90:3:0.3, 125 mg of pure title compound was obtained; MS; m/z (ES): 1152.7 [MH]⁺.

### Example 33

### 4″-O-{3-[2-(3-Carboxy-1-cyclopropyl-4-oxo-1,4-dihydro-quinolin-6-yloxy)ethoxy]propionyl}-6-O-methyl-11-desoxy-11-(R)-amino-erythromycin A11,12-carbamate

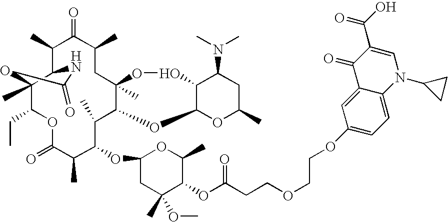

To a MeOH (25 mL) solution of Example 32 (50 mg), 10% Pd/C (10 mg) was added and the reaction mixture was shaken at 5 bar H₂ for 12 hours. The catalyst was filtered off and the solvent evaporated under reduced pressure. DCM and water were added to the residue and the pH value was adjusted to 9.5. The layers were separated and the water layer extracted with DCM (2×). The combined organic layers were washed with brine, dried over K₂CO₃ and evaporated yielding 43 mg of pure title compound; MS; m/z (ES): 1116.2 [MH]⁺.

### Example 34

### 4″-O-{3-[2-(3-Carboxy-7-chloro-1-cyclopropyl-4-oxo-1,4-dihydro-quinolin-6-yloxy)-ethoxy]-propionyl}-11-O-methyl-azithromycin

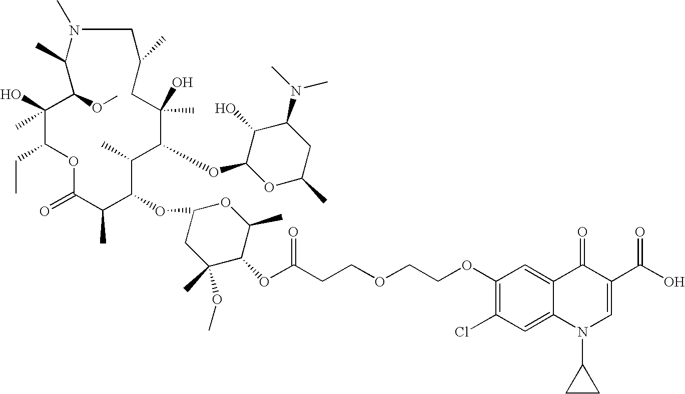

To a DCM (50 mL) solution of Intermediate 20 (1.0 g, 2.53 mmol), TEA (0.710 mL, 5.09 mmol) was added and the mixture was cooled to 0° C. under N₂ atmosphere. Pivaloyl chloride (0.63 mL, 5.11 mmol) was added to this mixture and the mixture was stirred for 1 hour at 0° C. Intermediate 22 (2.06 g, 2.56 mmol) and DMAP (0.94 g, 7.69 mmol) were then added and the reaction mixture was stirred at 0° C. to room temperature for 24 hours. Water was added to the reaction mixture and the layers were separated”. The water layer was extracted with DCM (2×) and the combined organic layers were washed with brine, dried over K₂CO₃, and evaporated under reduced pressure. Methanol (100 mL) was added to the residue and the reaction mixture was stirred for 24 hours at 65° C. Evaporation of methanol yielded 1.3 g of crude product which was precipitated twice from EtOAc/n-hexane yielding 840 mg of pure title product; MS; m/z (ES): 571.0 [MH₂]²⁺.

### Example 35

### 4″-O-{3-[2-(3-Carboxy-1-cyclopropyl-4-oxo-1,4-dihydro-quinolin-6-yloxy)-ethoxy]-propionyl}-11-O-methyl-azithromycin

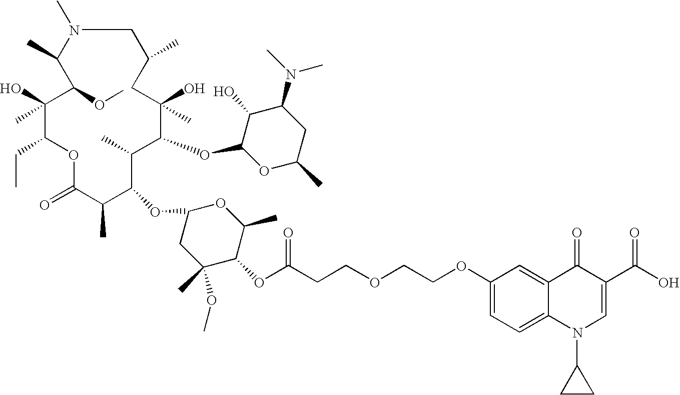

To a MeOH (30 mL) solution of Example 34 (410 mg, 0.359 mmol), 10% Pd/C (200 mg) was added and the reaction mixture was shaken at 5 bar for 5 hours. The catalyst was filtered off and the solvent evaporated under reduced pressure. DCM and water were added to the residue and the pH value was adjusted to 9.5. The layers were separated and the water layer was extracted with DCM (2×). The combined organic layers were washed with brine, dried over K₂CO₃ and evaporated yielding 216 mg of crude title product. The crude product was precipitated from EtOAc/n-hexane yielding 178 mg of pure title product; ¹³C NMR (MHz, CDCl₃) δ: 178.0, 171.3, 167.2, 157.3, 146.7, 135.7, 127.4, 124.9, 118.9, 108.1, 106.6, 102.3, 94.8, 85.1, 83.6, 79.2, 78.1, 77.9, 74.4, 73.2, 73.1, 71.1, 71.0, 69.3, 68.0, 67.8, 66.9, 65.5, 65.1, 63.0, 62.8, 62.7, 62.2, 49.5, 45.5, 42.8, 42.6, 40.5, 35.9, 35.5, 35.2, 35.1, 33.8, 27.7, 26.9 26.7, 22.2, 21.8, 21.7, 21.3, 17.7, 17.1, 14.7, 13.1, 14.7, 11.3, 9.3, 8.3, 7.2; MS; m/z (ES): 554.0 [MH₂]²⁺.

### Example 36

### 4″-O-{3-[2-(3-Carboxy-7-chloro-1-cyclopropyl-4-oxo-1,4-dihydro-quinolin-6-ylamino)ethoxy]propionyl}-6-O-methyl-erythromycin A

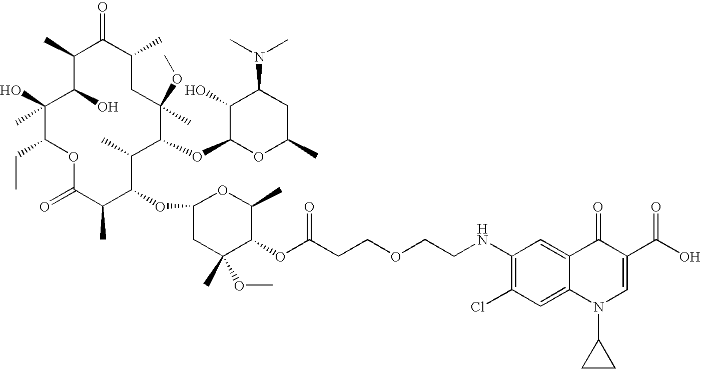

To a DCM (60 mL) solution of Intermediate 17 (1 g, 2.53 mmol), TEA (624 μL, 4.48 mmol) was added and the mixture was cooled to 0° C. under N₂ atmosphere. Pivaloyl chloride (552 μL, 4.48 mmol) was added to this mixture and the mixture was stirred for 1 hour at 0° C. 2′-O-Acetyl-6-O-methyl-erythromycin A (1 g, 1.27 mmol) and DMAP (928 mg, 7.6 mmol) were then added and reaction mixture was stirred at 0° C. to room temperature for 20 hours. Water was added to the reaction mixture and the layers were separated. The water layer was extracted with DCM (2×20 mL) and the combined organic layers were washed with brine, dried over K₂CO₃ and the solvent was evaporated under reduced pressure. Methanol (20 mL) was added to the residue and the reaction mixture was stirred for 20 hours at 65° C. Evaporation of methanol yielded 780 mg of product, which was purified on column yielding 440 mg of pure title product; ¹H-NMR (500 MHz, DMSO-d6) δ 8.73, (s, 1H), 8.05 (s, 1H), 7.54 (s, 1H), 5.06 (d, 3H), 5.00 (m, 3H), 4.99 (q, 3H), 4.71 (d, 1H), 4.57 (d, 1H), 4.35 (m, 1H), 3.82 (m, 7H), 3.81 (m, 7H), 3.75 (m, 7H), 3.64 (d, 1H), 3.57 (m, 1H), 3.48 (m, 3H), 3.30 (m, 3H), 3.21 (m, 2H), 3.02 (t, 4H), 2.99 (m, 1H), 2.91 (m, 1H), 2.64 (m, 4H), 2.57 (m, 4H), 2.55 (m, 4H), 2.44 (m, 7H), 2.30 (m, 6H), 1.95 (m, 3H), 1.91 (m, 3H), 1.83 (m, 3H), 1.71 (m, 3H), 1.66 (m, 3H), 1.63 (m, 3H), 1.48 (m, 6H), 1.40 (m, 6H), 1.36 (s, 4H), 1.21 (q, 6H), 1.18 (m, 28H), 1.19 (m, 28H), 1.14 (m, 28H), 1.13 (m, 28H), 1.12 (m, 28H), 1.10 (m, 28H), 0.84 (t, 3H); ¹³C-NMR (125 MHz, DMSO-d6) δ: 221.03, 177.56, 175.75, 171.11, 167.24, 146.01, 142.91, 132.74, 127.58, 126.33, 118.11, 107.73, 104.60, 102.08, 96.00, 80.43, 78.92, 78.31, 78.05, 76.67, 74.31, 72.70, 71.13, 59.13, 67.81, 65.32, 63.03, 50.69, 49.54, 45.36, 44.89, 43.30, 40.35, 39.23, 38.84, 37.24, 36.03, 36.02, 35.38, 35.24, 29.14, 21.85, 21.09, 21.07, 19.72, 18.37, 18.05, 15.99, 12.38, 10.62, 9.16, 8.15.

### Example 37

### 4″-O-{3-[2-(3-Carboxy-7-chloro-1 cyclopropyl-4-oxo-1,4-dihydro-quinolin-6-yloxy)ethoxy]propionyl}-6-O-methyl-erythromycin A

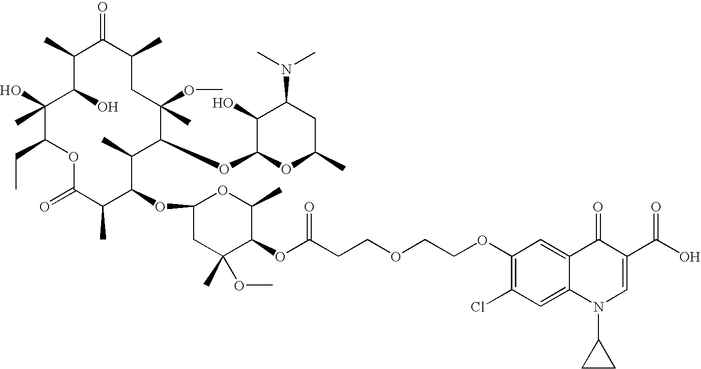

To a DCM (10 mL) solution of the Intermediate 20 (0.50 g), TEA (0.312 mL) was added and the mixture was cooled to 0° C. under N₂ atmosphere. Pivaloyl chloride (0.276 mL) was added to this mixture and the mixture was stirred for 1 hour at 0° C. 2′-O-Acetyl-6-O-methyl-erythromycin A (1.0 g) and DMAP (0.464 g) were then added and the reaction mixture was stirred at 0° C. to room temperature for 24 hours. Water was added to the reaction mixture and the layers were separated. The water layer was extracted with DCM (2×) and the combined organic layers were washed with brine, dried over K₂CO₃, evaporated under reduced pressure. Methanol (70 mL) was added to the residue and the reaction mixture was stirred for 24 hours at 65° C. Evaporation of methanol yielded 580 mg of crude product which was purified on a SPE-column yielding 100 mg of pure title product; MS; m/z (ES): 1126.7 [MH]⁺.

### Example 38

### 4″-O-{3-[2-(3-Carboxy-1-cyclopropyl-4-oxo-1,4-dihydro-quinolin-6-yloxy)ethoxy]propionyl}-6-O-methyl-erythromycin A

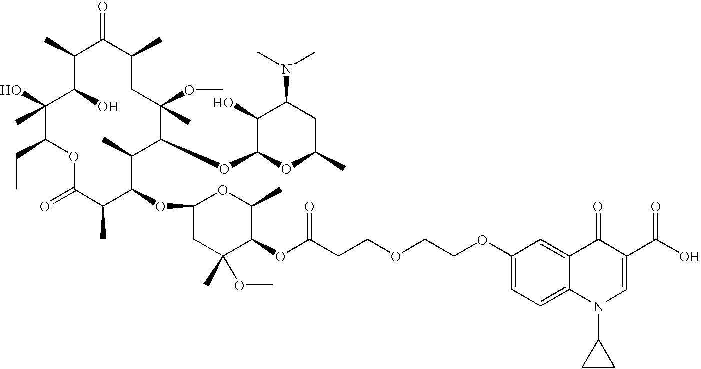

To a MeOH (10 mL) solution of Example 37 (150 mg), 10% Pd/C (200 mg) was added and the reaction mixture was shaken at 5 bar for 24 hours. The catalyst was filtered off and the solvent evaporated under reduced pressure. DCM and water were added to the residue and the pH value was adjusted to 9.5. The layers were separated and the water layer extracted with DCM (2×). The combined organic layers were washed with brine, dried over K₂CO₃ and evaporated yielding 95 mg of crude title product. After purification on a SPE column 50 mg of pure title compound was obtained; MS; m/z (ES): 1091.71 [MH]⁺.

### Example 39

### 4″-O-{3-[2-(3-Carboxy-1-cyclopropyl-4-oxo-1,4-dihydro-quinolin-6-ylamino)ethoxy]propionyl}-6-O-methyl-erythromycin A

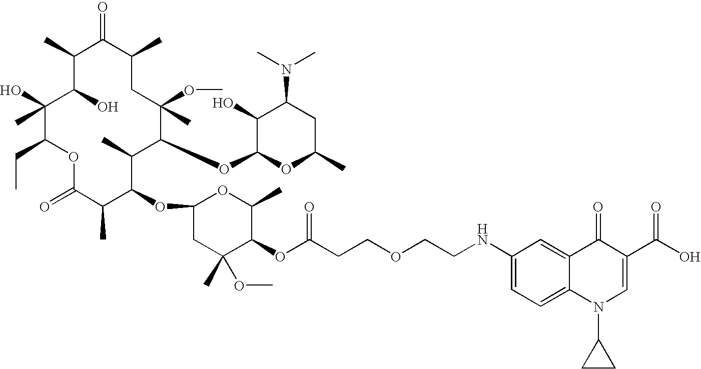

To a MeOH (10 mL) solution of Example 36 (100 mg), 10% Pd/C (100 mg) was added and the reaction mixture was shaken at 5 bar under hydrogen for 24 hours. The catalyst was filtered and the solvent evaporated under reduced pressure. DCM and water were added to the residue and the pH value was adjusted to 9.5. The layers were separated and the water layer extracted with DCM (2×). The combined organic layers were washed with brine, dried over K₂CO₃ and evaporated yielding 75 mg of crude title product. After purification on a SPE column 45 mg of pure title compound was obtained; MS; m/z (ES): 1089.73 [MH]⁺.

### Example 40

### 4″-O-{3-[2-(3-Carboxy-1-cyclopropyl-6-fluoro-4-oxo-1,4-dihydro-quinolin-7-ylamino)ethoxy]propionyl}-azithromycin

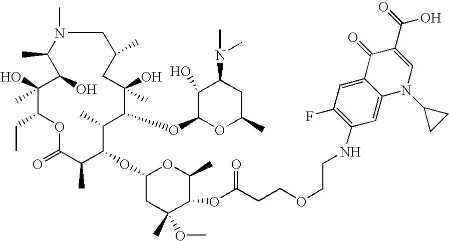

A DMF (5 mL) solution of crude Intermediate-21 (0.420 g, 1.106 mmol) was cooled to 0° C. To this mixture EDC.HCl (1.0 g, 5.216 mmol) was added and the reaction mixture was stirred at 0° C. to room temperature for 1 hour. 2′-O-Acetyl-azithromycin (1.0 g, 1.266 mmol) and DMAP (0.27 g, 2.21 mmol) were then added and the reaction mixture was stirred at 0° C. to room temperature for 44 hours. Water and EtOAc were added and the layers were separated. The water layer was extracted twice with EtOAc. The combined organic layers were dried over K₂CO₃ and evaporated yielding 1.25 g of crude 2′-protected product in a mixture with starting compounds (incomplete conversion). This product was dissolved in MeOH (60 mL) and the solution was stirred for 24 hours at 65° C. and for 3 days at room temperature. The methanol was evaporated under reduced pressure and the residue (1.15 g) was precipitated form EtOAc:n-hexane yielding 0.6 g crude product which was purified by column chromatography (DCM:MeOH:NH₃=90:4:0.5) yielding which was precipitated form EtOAc:n-hexane yielding 9.82 mg of pure title product; MS; m/z (ES): 1109.00 [MH]⁺.

### Example 41

### 4″-O-{3-[2-(3-Carboxy-1-cyclopropyl-6-fluoro-8-methoxy-4-oxo-1,4-dihydro-quinolin-7-ylamino)ethoxy]propionyl}-azithromycin

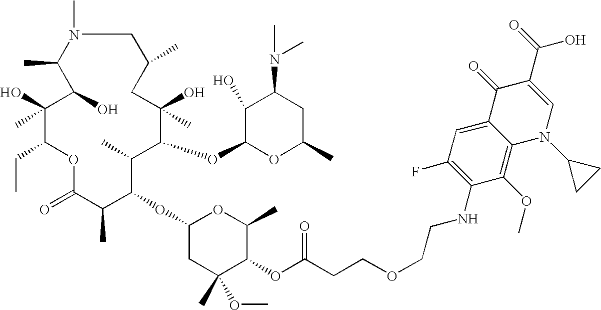

EDC.HCl (0.483 g) was added to a suspension of Intermediate 25 (0.336 g) in DMF (2.4 mL) and the mixture was cooled to 0° C. under a N₂ atmosphere. A solution of 2′-O-acetyl-azithromycin (0.5 g) in DCM (1.5 mL) was dropped into the mixture and DMAP (0.135 g) was added. The resulting mixture was stirred from 0° C. up to room temperature under N₂ overnight. Water was added to the reaction mixture and the layers were separated. The aqueous layer was extracted with EtOAc (2×10 mL) and the combined organic layers were washed with brine, dried over K₂CO₃ and the solvent was evaporated under pressure. The crude product was dissolved in MeOH (50 mL) and stirred at 50° C. for 24 h. Solvent evaporation under reduced pressure gave the title compound (195 mg). Column chromatography in 90:9:1.5 (DCM, MeOH:NH₃) yielded 135 mg of the title compound. MS (ES+)m/z: [MH]⁺=1139.

### Example 42

### 4″-O-{3-[2-(3-Carboxy-6-fluoro-8-methoxy-1-cyclopropyl-4-oxo-1,4-dihydro-quinolin-7-ylamino)-ethoxy]-propionyl}-azithromycin11,12-carbonate

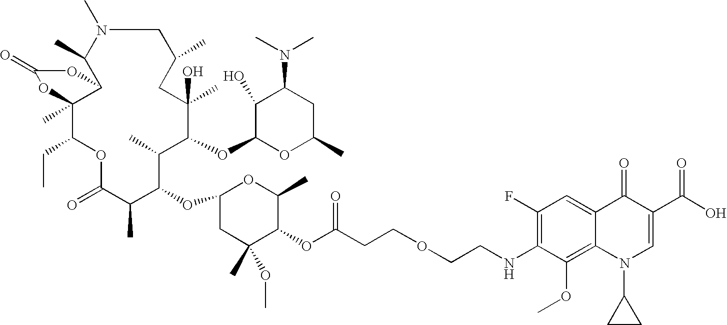

EDC.HCl (0.514 g) was added to a suspension of Intermediate 25 (0.649 g) in DMF (7 mL) and the mixture was cooled to 0° C. under a N₂ atmosphere. A solution of 2′-O-acetyl-azithromycin-11,12-carbonate (1 g) in DCM (4 mL) was dropped into the mixture and DMAP (0.269 g) was added. The resulting mixture was stirred from 0° C. up to room temperature under N₂ overnight. Water was added to the reaction mixture and the layers were separated. The aqueous layer was extracted with EtOAc (2×10 mL) and the combined organic layers were washed with brine, dried over K₂CO₃ and the solvent was evaporated under pressure. The crude product was dissolved in MeOH (50 mL) and stirred at 50° C. for 24 h. Solvent evaporation under reduced pressure gave the title compound. Purification by column chromatography (spe column; eluent: DCM:MeOH:NH₃=90:15:1.5) yielded 0.650 g of pure title compound. MS(ES+)m/z: [MH]⁺=1165.39.

### Example 43

### 4″-O-{3-[2-(3-Carboxy-6-fluoro-8-methoxy-1-cyclopropyl-4-oxo-1,4-dihydro-quinolin-7-ylamino)ethoxy]-propionyl}-11-O-methylazithromycin

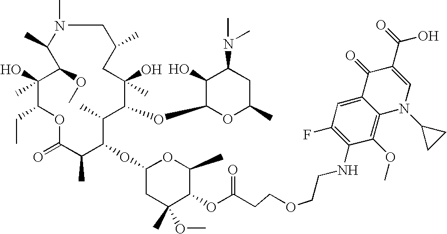

EDC.HCl (0.783 g) was added to a suspension of Intermediate 25 (0.98 g) in DMF (7 mL) and the mixture was cooled to 0° C. under a N₂ atmosphere. A solution of Intermediate 22 (1.5 g) in DCM (4 mL) was dropped into the mixture and DMAP (0.389 g) was added. The resulting mixture was stirred from 0° C. up to room temperature under N₂ overnight. Water was added to the reaction mixture and the layers were separated. The aqueous layer was extracted with EtOAc (2×10 mL) and the combined organic layers were washed with brine, dried over K₂CO₃ and the solvent was evaporated under reduced pressure. The crude product was dissolved in MeOH (50 mL) and stirred at 50° C. for 24 h. Solvent evaporation under reduced pressure gave the title compound. Purification by column chromatography (spe column; eluent: DCM:MeOH:NH₃=90:15:1.5) yielded 0.280 g of pure title compound. MS (ES+)m/z: [MH]⁺=1153.39.

### Example 44

### 4″-O-{3-[2-(3-Carboxy-6-fluoro-8-methoxy-1-cyclopropyl-4-oxo-1,4-dihydro-quinolin-7-ylamino)-ethoxy]-propionyl}-clarithromycin

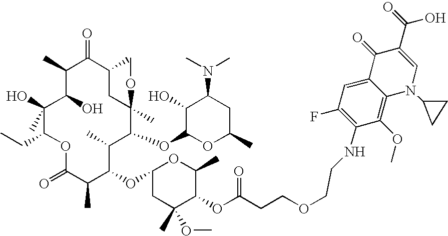

EDC.HCl (0.483 g) was added to a suspension of Intermediate 25 (0.672 g) in DMF (5 mL) and the mixture was cooled to 0° C. under a N₂ atmosphere. A solution of 2′-O-acetyl-clarithromycin (1 g) in DCM (3 mL) was dropped into the mixture and DMAP (0.231 g) was added. The resulting mixture was stirred from 0° C. up to room temperature under a N₂ overnight. Water was added to the reaction mixture and the layers were separated. The water layer was extracted with EtOAc (2×10 mL) and the combined organic layers were washed with brine, dried over K₂CO₃ and the solvent was evaporated under pressure. The crude product was dissolved in MeOH (50 mL) and stirred at 50° C. for 24 h. Solvent evaporation under reduced pressure gave the title compound. Purification by column chromatography (spe column; eluent: DCM:MeOH:NH₃=90:15:1.5) yielded 0.181 g of pure title compound. MS (ES+)m/z: [MH]⁺=1138.6.

### Example 45

### 4″-O-{3-[2-(3-Carboxy-7-chloro-1-cyclopropyl-4-oxo-1,4-dihydro-quinolin-6-ylamino)ethoxy]-propionyl}-2′-O-propionyl-azithromycin

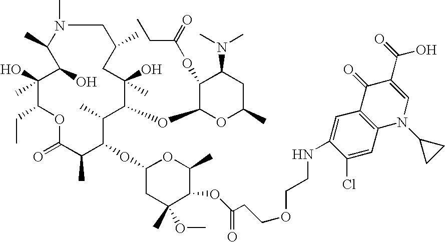

To a solution of Example 18 (200 mg) in DCM (20 mL), sodium hydrogen carbonate (68.7 mg) and propionic acid anhydride (28.4 μL) were added and the mixture was stirred at room temperature overnight. DCM (20 mL) was added to the reaction mixture and the mixture was extracted with water (3×20 mL). The organic layers were washed with brine and the solvent was concentrated in vacuo affording 200 mg of the title compound. MS (ES) m/z:[MH]⁺ 1126.8.

### Example 46

### 4″-O-{3-[2-(3-Carboxy-1-cyclopropyl-4-oxo-1,4-dihydro-quinolin-6-ylamino)-ethoxy]-propionyl}-2′-O-propionyl-azithromycin

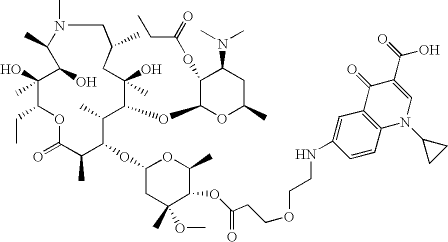

To a solution of Example 19 (200 mg) in DCM (20 mL), sodium hydrogen carbonate (70 mg) and propionic acid anhydride (28.3 μL) were added and the mixture was stirred at room temperature overnight. DCM (20 mL) was added to the reaction mixture and the mixture was extracted with water (3×20 mL). The organic layers were washed with brine and the solvent was concentrated in vacuo affording 190 mg of the title compound. MS (ES) m/z: [MH]⁺ 1148.42.

### Example 47

### 4″-O-[3-[2-[(3-Carboxy-7-chloro-1-cyclopropyl-1,4-dihydro-4-oxo-6-quinolinyl)-amino]ethoxy]propionyl]-6-O-methyl-9(E)-O-ethyloximino erythromycin A

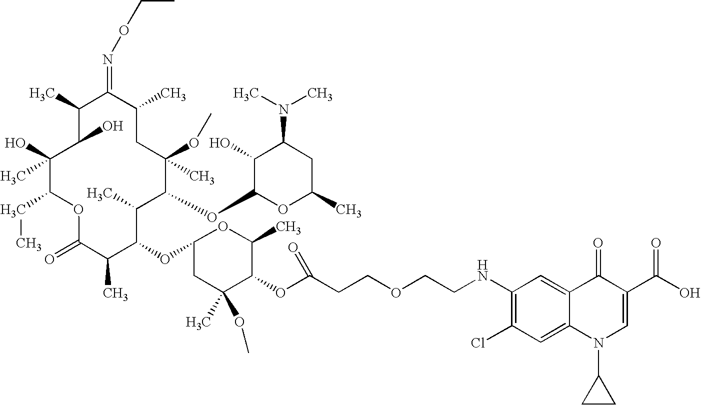

Intermediate 17 (100 mg, 0.25 mmol) was dissolved in dry DMF (5 mL) and the solution was cooled in an ice bath under N₂. EDC.HCl (97 mg, 0.5 mmol) was added and the resulting mixture was stirred for 5 min. A dry DCM (5 mL) solution of Intermediate 31 (422 mg, 0.5 eq) was added followed by addition of DMAP (93 mg, 0.75 mmol). The mixture was then stirred at room temperature for 48 hours. Water (10 mL) and DCM (10 mL) were added, the aqueous layer was extracted with an additional 5 mL of DCM and the combined organic layers were dried over K₂CO₃, filtrated and evaporated. The residue was dissolved in MeOH (15 mL) and the solution was stirred at room temperature for 48 hours. The MeOH was then evaporated, DCM (10 mL) and water (10 mL) were added, and the DCM layer washed one more time with water (5 mL), dried over K₂CO₃ and evaporated. The residue was purified by column chromatography (DCM:MeOH:aq. NH₃=90:9:1.5). The chromatographically homogenous fractions were combined and evaporated. Precipitation by ethyl acetate-n-hexane gave the title compound (12 mg). MS m/z 1168.3(M+H)⁺.

### Example 48

### 4″-O-[3-[2-[(3-Carboxy-7-chloro-1-cyclopropyl-1,4-dihydro-4-oxo-6-quinolinyl)-amino]ethoxy]propionyl]-9(E)-O-methoxyimino erythromycin A

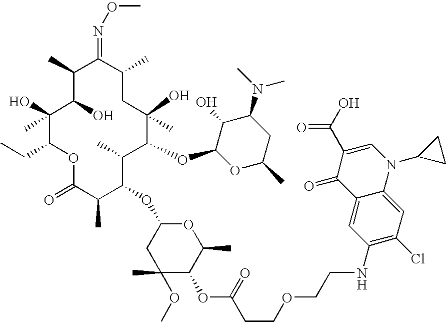

EDC.HCl (500 mg, 2.6 mmol) was added to a DMF/dry (3 mL) solution of Intermediate 17 (690 mg, 1.7 mmol) cooled on ice bath and the reaction mixture was stirred at 0° C. for ˜30 min under the flow of N₂. Intermediate 27 (700 mg, 0.87 mmol) and DMAP (200 mg, 1.6 mmol were then added. The resulting mixture was stirred for 24 h, during which time the reaction mixture was allowed to warm to ambient temperature. Water (10 mL) and DCM (15 mL) were then added to the mixture and the layers were separated. The water layer was extracted twice with DCM. The organic layers were collected, dried on Na₂SO₄, filtered and the organic solvent was evaporated. The residue was dissolved in MeOH (50 mL) and solution was stirred overnight at 60° C. on oil bath. The methanol was evaporated under vacuum and the foamy residue was purified by column chromatography (DCM:MeOH:NH₃=90:5:0.5). The product was precipitated from ethyl acetate-hexane yielding 115 mg of the title compound. ESMS m/z 1140 [MH³⁰ ].

### Example 49

### 4″-O-[3-[2-[(3-Carboxy-7-chloro-1-cyclopropyl-1,4-dihydro-4-oxo-6-quinolinyl)-amino]ethoxy]propionyl]-9(E)-O-(2-propyl)oximino erythromycin A

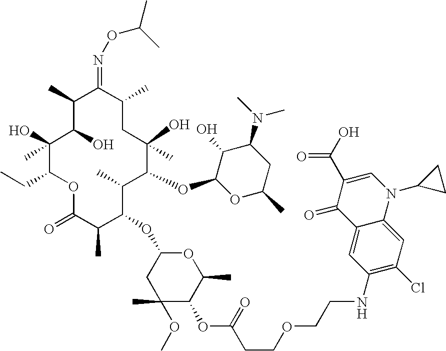

In an analogous procedure to that of Example 48, Intermediate 28 gave the title compound (101 mg) as a yellow solid. ESMS m/z 1168 [MH⁺].

### Example 50

### 4″-O-{3-[2-(1-Cyclopropyl-3-isopropoxycarbonyl-4-oxo-1,4-dihydro-quinolin-6-yloxy)-ethoxy]-propionyl}azithromycin

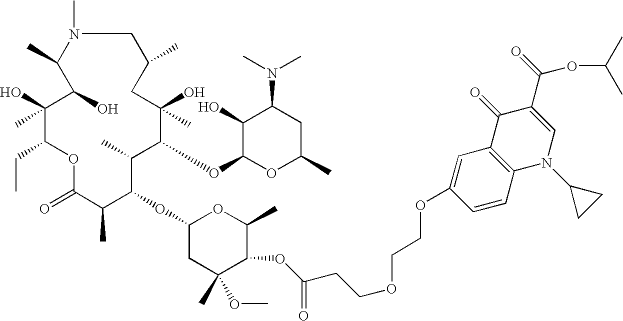

To a solution of Example 29 (0.4 g) in DMF (10 mL), were added K₂CO₃ (1.32 g), BTEAC (0.084 g) and 2-propanol (0.073 mL), and the reaction was stirred at room temperature for 24 hours. Water and EtOAc were added to the reaction mixture and the layers were separated. The aqueous layer was extracted with EtOAc. The combined organic extracts were evaporated yielding 420 mg of crude product. Purification by column chromatography (SPE-column, gradient polarity: 100% DCM to DCM:MeOH:NH₃=90:9:0.5) yielded 270 mg of the title compound which then was precipitated from EtOAc:n-hexane yielding 200 mg of pure title compound, MS; m/z (ES): 1135.81 [MH]⁺.

### Example 51

### 4″-O-{3-[2-(7-Chloro-1-cyclopropyl-3-isopropoxycarbonyl-oxo-1,4-dihydro-quinolin-6-ylamino)-ethoxy]propionyl}-azithromycin

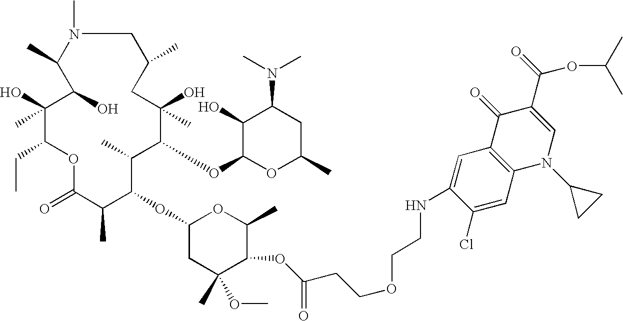

To a solution of Example 18 (0.4 g) in DMF (10 mL), were added K₂CO₃ (1.28 g), BTEAC (0.081 g) and 2-propanol (0.071 mL), and the reaction was stirred at room temperature for 23 hours (conversion was about 50%). Water and EtOAc were added to the reaction mixture and the layers were separated. The aqueous layer was extracted with EtOAc. The combined organic extracts were evaporated yielding 420 mg of crude product. Purification by column chromatography (SPE-Column, gradient polarity: 100% DCM to DCM:MeOH:NH₃=90:9:0.5) yielded 100 mg of product which was precipitated from EtOAc:n-hexane yielding 80 mg of pure title compound. MS; m/z (ES): 1169.08 [MH]⁺.

### Example 52

### 4″-O-{3-[2-(1-Cyclopropyl-3-ethoxycarbonyl-4-oxo-1,4-dihydro-quinolin-6-ylamino)-ethoxy]-propionyl}-azithromycin

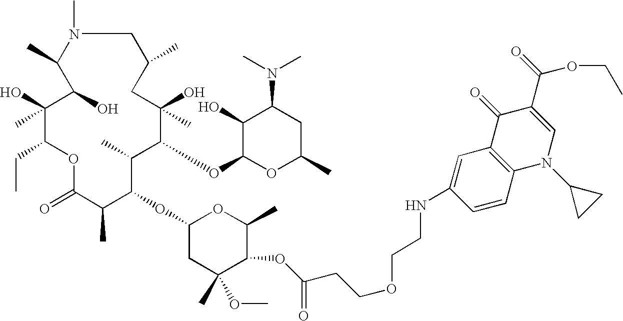

To a solution of Example 19 (0.4 g) in DMF (10 mL), were added K₂CO₃ (1.32 g), BTEAC (0.084 g) and iodoethane (0.060 mL), and the reaction was stirred at room temperature for 20 hours. Water and EtOAc were added to the reaction mixture and the layers were separated. The aqueous layer was extracted with EtOAc. The combined organic extracts were evaporated yielding 390 mg of crude product. Purification by column chromatography (SPE-column, gradient polarity: 100% DCM to DCM:MeOH:NH3=90:9:0.5) yielded 220 mg of product which was precipitated from EtOAc:n-hexane yielding 160 mg of pure title compound. MS; m/z (ES): 1119.80 [MH]⁺.

### Example 53

### 4″-O-{3-[2-(1-Cyclopropyl-3-isopropoxycarbonyl-4-oxo-1,4-dihydro-quinolin-6-ylamino)-ethoxy]-propionyl}-azithromycin

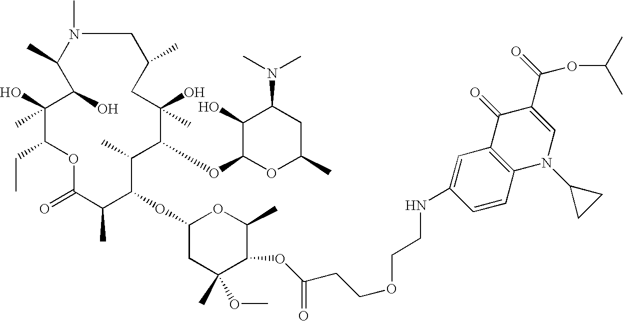

To a solution of Example 19 (0.3 g) in DMF (10 mL), were added K₂CO₃ (0.99 g), BTEAC (0.063 g) and 2-propanol (0.055 mL), and the reaction was stirred at room temperature for 20 hours. Water and EtOAc were added to the reaction mixture and the layers were separated. The aqueous layer was extracted with EtOAc. The combined organic extracts were evaporated yielding 410 mg of crude product. Purification by column chromatography (SPE-column, gradient polarity: 100% DCM to DCM:MeOH:NH₃=90:9:0.5) yielded 180 mg of product which was precipitated from EtOAc:n-hexane yielding 130 mg of pure title compound. MS; m/z (ES): 1133.40 [MH]⁺.

### Example 54

### 4″-O-{3-[2-(1-Cyclopropyl-3-propoxycarbonyl-4-oxo-1,4-dihydro-quinolin-6-ylamino)ethoxy]-propionyl}-azithromycin

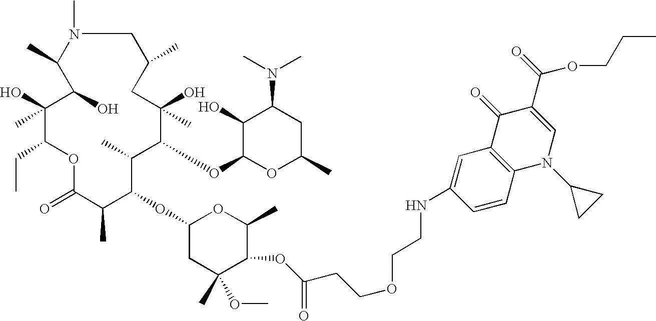

To a solution of Example 19 (0.4 g) In DMF (10 mL), were added K₂CO₃ (1.32 g), BTEAC (0.084 g) and 2-propanol (0.072 mL), and the reaction was stirred at room temperature for 20 hours. Water and EtOAc were added to the reaction mixture and the layers were separated. The aqueous layer was extracted with EtOAc. The combined organic extracts were evaporated yielding 400 mg of crude product. Purification by column chromatography (SPE-column, gradient polarity: 100% DCM to DCM:MeOH:NH₃=90:9:0.5) yielded 190 mg of product which was precipitated from EtOAc:n-hexane yielding 150 mg of pure title compound. MS; m/z (ES): 1133.96 [MH]⁺.

### Example 55

### 4″-O-{3-[2-(7-Chloro-1-Cyclopropyl-3-isopropoxycarbonyl-4-oxo-1,4-dihydro-quinolin-6-yloxy)-ethoxy]-propionyl}azithromycin

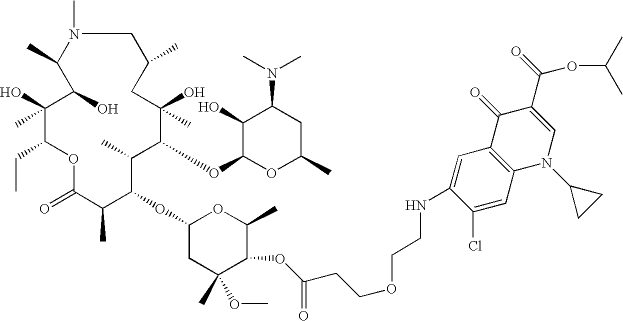

To a solution of Example 27 (3.0 g) in DMF (100 mL), were added K₂CO₃ (9.55 g), BTEAC (0.61 g) and 2-propanol (0.8 mL), and the reaction mixture was stirred at room temperature for 20 hours. Additional 2-propanol (0.2 mL) was then added and the reaction mixture was stirred at room temperature for a further 4 hours. Water and EtOAc were added to the reaction mixture and the layers were separated. The aqueous layer was extracted with EtOAc. The combined organic extracts were evaporated yielding 2.7 g of crude product. Purification by column chromatography (eluent: DCM:MeOH:NH₃=90:5:0.5) yielded 2.0 g of product which was precipitated from EtOAc:n-hexane yielding 1.71 of pure title compound. MS; m/z (ES): 1168.81 [MH]⁺.

### Example 56

### 4″-(3-{2-[3-(3-Benzyloxy-propoxycarbonyl)-1-cyclopropyl-4-oxo-1,4-dihydro-quinolin-6-ylamino]-ethoxy}propionyl)-azithromycin

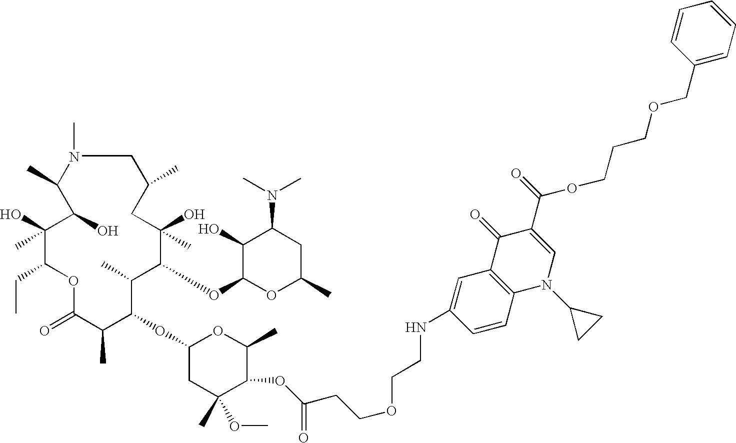

To a solution of Example 19 (0.2 g) in DMF (5 mL), was added K₂CO₃ (0.05 g) and the reaction mixture was stirred for 1.5 hour at room temperature. To this reaction mixture benzyl-3-bromopropyl ether (0.064 mL) was added and the reaction mixture was stirred for a further 8 hours at room temperature. Only 50% conversion was registered, so BTEAC (0.042 g) was added and the reaction was stirred at 40° C. for 24 hours. Water and EtOAc were added to the reaction mixture and the layers were separated. The aqueous layer was extracted with EtOAc. The combined organic extracts were evaporated yielding 0.21 g of crude product. Purification by column chromatography (eluent: DCM:MeOH:NH₂=90:5:0.5) yielded 0.14 g of product which was precipitated from EtOAc:n-hexane yielding 80 mg of pure title compound. MS; m/z (ES): 1240.2 [MH]⁺.

### Example 57

### 4″-O-(3-{2-[1-Cyclopropyl-3-(1-ethoxycarbonyloxy-ethoxycarbonyl)-4-oxo-1,4-dihydro-quinolin-6-ylamino]-ethoxy}-propionyl)-azithromycin

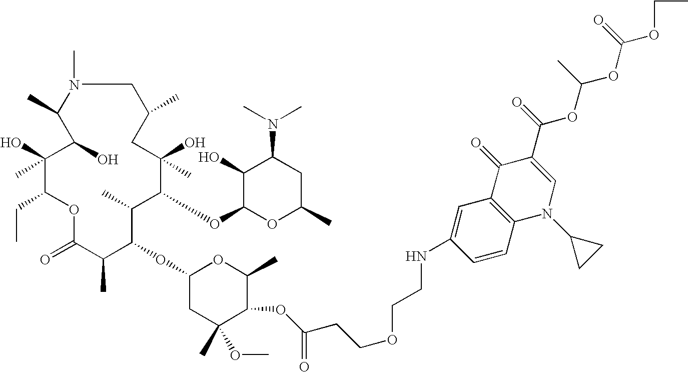

To a solution of Example 19 (0.5 g) in DMF (10 mL), was added K₂CO₃ (0.82 g) and the reaction mixture was stirred for 2 hours at room temperature. To this reaction mixture were added 1-chloro-ethyl-ethylcarbonate (0.62 mL) and BTEAC (0.32 g), and the reaction mixture was stirred for 16 hours at room temperature. Water and EtOAc were added to the reaction mixture and the layers were separated. The aqueous layer was extracted with EtOAc. The combined organic extracts were evaporated and then precipitated from EtOAc:n-hexane yielding 0.33 g of crude product. Purification by column chromatography (eluent DCM:MeOH:NH₃=90:3:0.5) yielded 110 mg of product which was precipitated from EtOAc:n-hexane yielding 100 mg of pure title compound. MS; m/z (ES): 1208.73 [MH]⁺.

### Example 58

### 4″-O-(3-{2-[7-Chloro-1-chloropropyl-4-oxo-3-(2-oxo-propoxycarbonyl)-1,4-dihydro-quinolin-6-yloxy]-ethoxy}-propionyl)-11-O-methylazithromycin

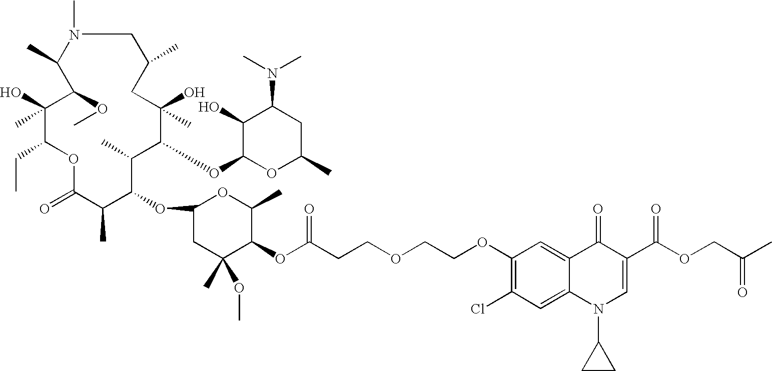

To a solution of Example 34 (200 mg) in DMF (5 mL), were added 1-chloro-propan-2-one (0.02787 mL), K₂CO₃ (0.072 g) and BTEAC (0.039 g). The reaction mixture was stirred at room temperature for 20 hours and then extracted with EtOAc (2×20 mL) and water (2×20 mL). The organic layer was dried over K₂CO₃ and evaporated in vacuo. The product was precipitated from EtOAc:diisopropyl-ether yielding 0.100 g of the title compound. MS (ES+)m/z: [MH]⁺=1196.90.

### Example 59

### 4″-O-(3-{2-[1-Cyclopropyl-3-(3-dimethylamino-propoxycarbonyl)-4-oxo-1,4-dihydro-quinolin-6-ylamino]-ethoxy}-propionyl)-azithromycin

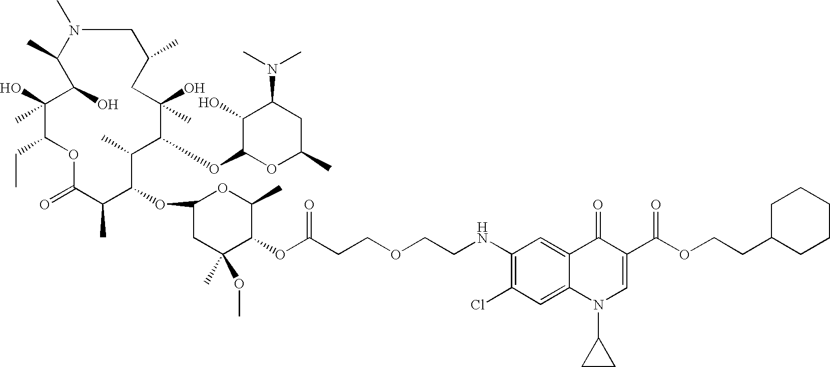

To a solution of Example 18 (400 mg) in DMF (10 mL), was added K₂CO₃ (640 mg). After 2 h 1-bromo-2-cyclohexylethane was added and the reaction mixture was heated at 50° C. and stirred overnight. 60 mL of H₂O was then added and the mixture was extracted with 3×30 ml EtOAc. The organic layer washed with NaCl (50 mL), dried over K₂CO₃ and evaporated in vacuo. The product was purified by SPE-chromatography (eluent: CH₂Cl₂:MeOH:NH₃=90:3:0.5). Precipitation from EtOAc:n-hexane yielded 270.51 mg of the title compound. MH³⁰ =1237.4.

### Example 60

### 4″-O-{3-[2-(1-Cyclopropyl-3-ethoxycarbonylmethoxycarbonyl-4-oxo-1,4-dihydro-quinolin-6-ylamino)-ethoxy]propionyl}-azithromycin

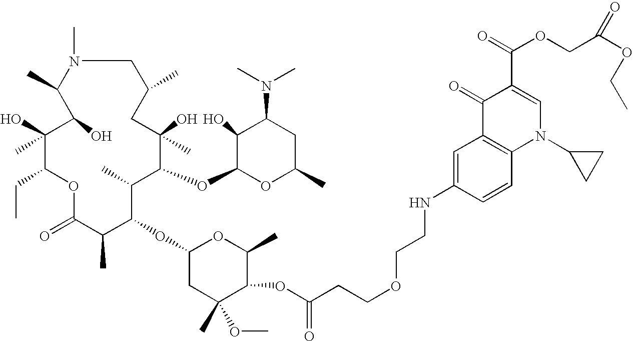

Example 19 (400 mg) was dissolved in DMF/4 Å (10 mL). K₂CO₃ (152 mg) and BTEAC (84 mg) were added to the solution and the resulting mixture was stirred for 1 h. Ethylbromoacetate (123 μL) was then added and the mixture was stirred for 4 h at room temperature. EtOAc (20 mL) was added and the mixture was extracted with H₂O (3×30 mL). The organic layers were washed with brine, dried over Na₂SO₄, filtered and the solvent was concentrated in vacuo affording 200 mg of product. Column chromatography in 90:3:0.5 (DCM:MeOH:NH₃) yielded 100 mg of the title compound. MS (ES) m/z:[MH]⁺ 1178.45.

### Example 61

### 4″-O-(3-{2-[1-Cyclopropyl-3-(3-dimethylamino-propoxycarbonyl)-4-oxo-1,4-dihydro-quinolin-6-ylamino]-ethoxy}-propionyl)-azithromycin

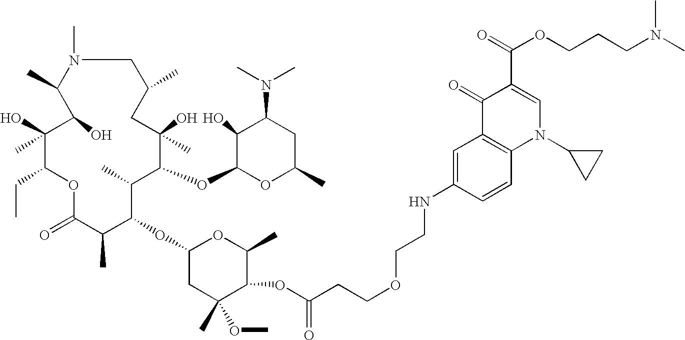

Example 19 (400 mg) was dissolved in DMF/4 Å (10 mL). K₂CO₃ (154 mg) and BTEAC (84 mg) were added to the solution and the resulting mixture was stirred for 1 h. 3-Dimethylaminopropylchloride (117 mg) was then added and the mixture was stirred for 4 h at room temperature. EtOAc (20 mL) was added and the mixture was extracted with H₂O (3×30 mL). The organic layers were washed with brine, dried over Na₂SO₄, filtered and the solvent was concentrated in vacuo affording 250 mg of product Column chromatography in 90:3:0.5 (DCM:MeOH:NH₃) yielded 110 mg of the title compound. MS (ES) m/z: [MH]⁺ 1177.52.

### Example 62

### 4″-O-(3-{2-[1-Cyclopropyl-3-methoxycarbonyl-4-oxo-1,4-dihydro-quinolin-6-ylamino]-ethoxy}-propionyl)-azithromycin

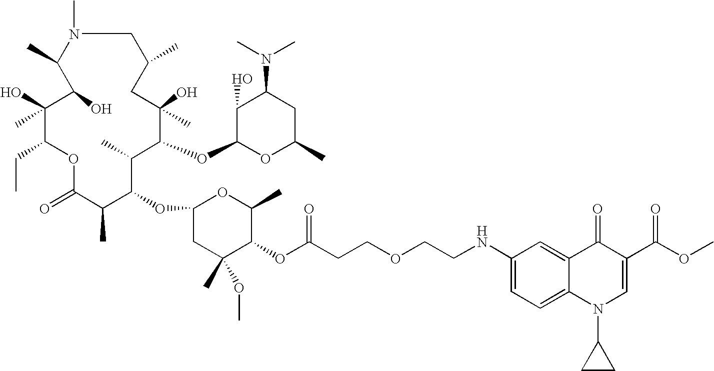

A solution of diazomethane in diethylether (60 mL) was added to a solution of Example 19 (1 g) in methanol (10 mL) and the resulting mixture was stirred overnight at ambient temperature. TLC showed no starting material and 1.03 g of crude product was obtained. HPLC/MS analysis confirmed the mass of the title product (m/z=1106.4 (MH⁺)). The product was purified by column chromatography.

### Example 63

### 4″-O-(3-{2-[1-Cyclopropyl-4-oxo-3-(2-pyrrolidin-1-yl-ethoxycarbonyl)-1,4-dihydro-quinolin-6-ylamino]ethoxy}-propionyl)-azithromycin

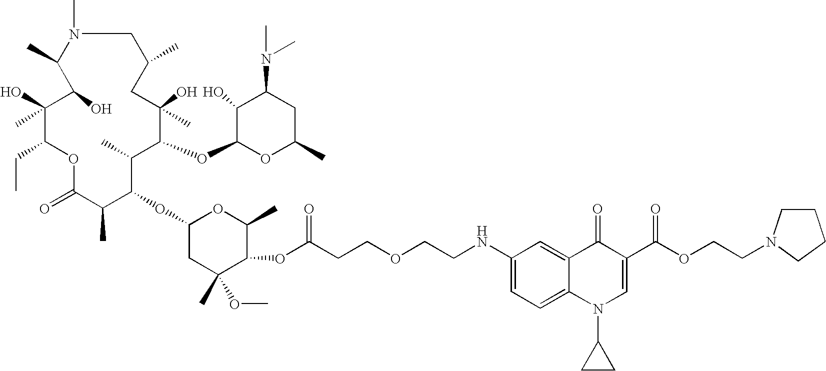

To a solution of Example 19 (2 g, 1.8 mmol) in DMF (50 mL), K₂CO₃ (3.2 g, 23,09 mmol) was added and the reaction mixture was stirred at room temperature for 90 minutes. N-(2-Chloroethyl)-dibenzyl-amine hydrochloride (1.58 g, 5.3 mmol) and BTEAC (0.405 g) were then added and stirring was continued for 24 hours at 40° C. Another portion of K₂CO₃ (5 eq.). N-(2-chloroethyl)-dibenzyl-amine hydrochloride (3 eq.) and BTEAC (1 eq.) were added and the reaction mixture was stiffed for another 24 hours at 40° C., then diluted with water (200 mL) and extracted with EtOAc (3×80 mL). The combined organic layers were washed with brine (2×50 mL), dried and evaporated in vacuo. The product was purified by column chromatography (fraction, eluent: CH₂Cl₂:MeOH:NH₃=90:3:0.5). Precipitation from EtOAc:n-hexane yielded 0.445 g of 100% pure title compound and 0.385 g of title compound. MS: m/z (ES): 1348.5 [MH]⁺.

### Example 64

### 4″-O-(3-{2-[1-Cyclopropyl-3-(2-dibenzylamino-ethoxycarbonyl)-4-oxo-1,4-dihydro-quinolin-6-ylamino]-ethoxy}-propionyl)-azithromycin

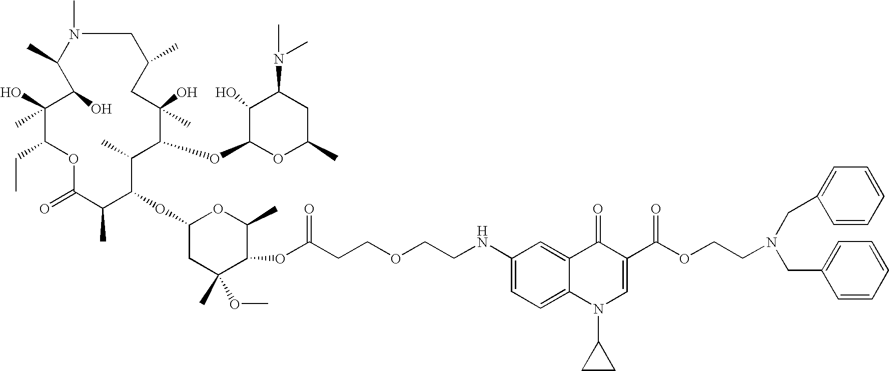

To a solution of Example 19 (0.4 g) in DMF (10 mL). K₂CO₃ (0.66 g, 13 eq) was added. After 90 minutes, 0.084 g BTEAC (1 eq) and N-(2-chloroethyl)dibenzyl-amine hydrochloride (3 eq) were added and the reaction mixture was stirred at 40° C. After 4 hours, 5 eq of K₂CO₃, 1 eq of BTEAC and 3 eq of amine hydrochloride were added and the reaction mixture was stirred at 40° C. for another 18 hours, then diluted with water (100 mL) and extracted with EtOAc (3×25 mL). The combined organic layers were washed with brine (2×20 mL) and aqueous NaHCO₃ (30 mL), then dried and evaporated. Crude product was first precipitated from EtOAc:n-hexane and then purified by column chromatography (eluent CH₂Cl₂:MeOH:NH₃=90:3:0.5) yielding 0.114 g of crude title compound. MS; m/z (ES): 1091.3 [MH]⁺ (1314.6-224.2).

### Example 65

### 4″-O-(3-{2-[1-Cyclopropyl-3-[(ethoxycarbonylmethyl-carbamoyl)-methoxycarbonyl]-4-oxo-1,4-dihydro quinolin-6-ylamino]-ethoxy}-propionyl)-azithromycin

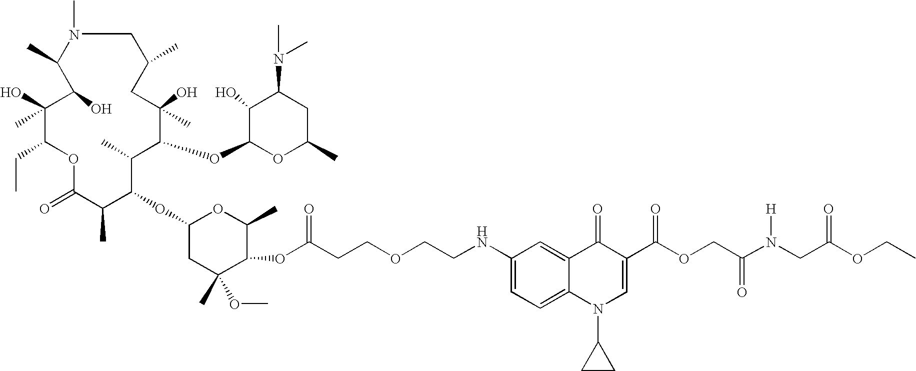

To a solution of Example 19 (0.3 g) in DMF (6 mL), K₂CO₃ (0.5 g, 13 eq) was added. After 90 minutes, 0.063 g (1 eq) BTEAC and 0.15 g (3 eq) of N-(chloroacetyl)glycine ethyl ester were added and the reaction mixture was stirred at 40° C. for 20 hours, then diluted with H₂O (50 mL) and extracted with EtOAc (2×20 mL). The combined organic layers were washed with brine (2×25 mL) and aqueous NaHCO₃ (25 mL) and then evaporated in vacuo. Crude product was first precipitated from EtOAc:n-hexane and then purified by column chromatography (SPE column, eluent CH₂Cl₂:MeOH:NH₃=90:5:0.5) yielding 0.134 g of white title compound. MS; m/z (ES): 1234.5 [MH]⁺.

### Example 66

### 4″-O-(3-{2-[7-Chloro-1-cyclopropyl-3-(2-dibenzylamino-ethoxycarbonyl)-4-oxo-1,4-dihydro-quinolin-6-ylamino]-ethoxy}-propionyl)-azithromycin

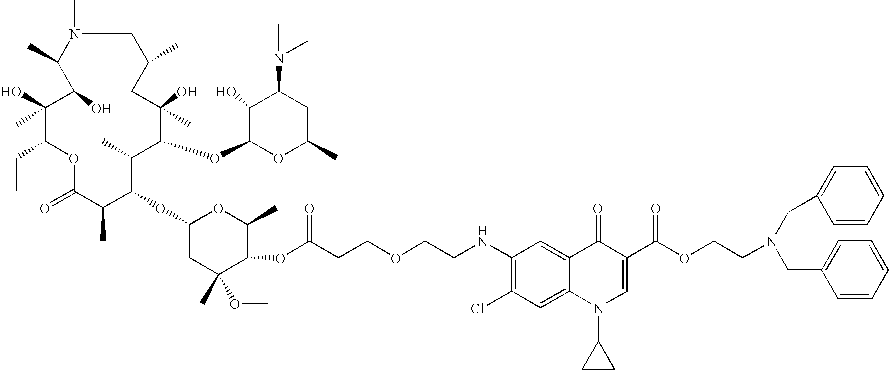

To a solution of Example 18 (2 g, 1.8 mmol) in DMF (50 mL), K₂CO₃ (3.2 g, 23.0 g mmol) was added and the reaction mixture was stirred at room temperature for 90 minutes. N-(2-Chloroethyl)-dibenzyl-amine hydrochloride (1.58 g, 5.3 mmol) and BTEAC (0.405 g) were then added and stirring was continued for 24 hours at 40° C. Another portion of K₂CO₃ (5 eq.). N-(2-chloroethyl)-dibenzyl-amine hydrochloride (3 eq.) and BTEAC (1 eq.) were then added and the reaction mixture was stirred for another 24 hours at 40° C., then diluted with water (200 mL) and extracted with EtOAc (3×80 mL). The combined organic layers were washed with brine (2×50 mL), dried and evaporated in vacuo. The product was purified by column chromatography (fraction, eluent: CH₂Cl₂:MeOH:NH₃=90:3:0.5). Precipitation from EtOAc:n-hexane yielded 0.445 g of 100% pure title compound and 0.385 g of title compound. MS; m/z (ES): 1348.5 [MH]⁺.

### Example 67

### 4″-O-(3-{2-[7-Chloro-1-cyclopropyl-4-oxo-3-(2-pyrrolidin-1-yl-ethoxycarbonyl)-1,4-dihydro-quinolin-6-ylamino]-ethoxy}-propionyl)-azithromycin

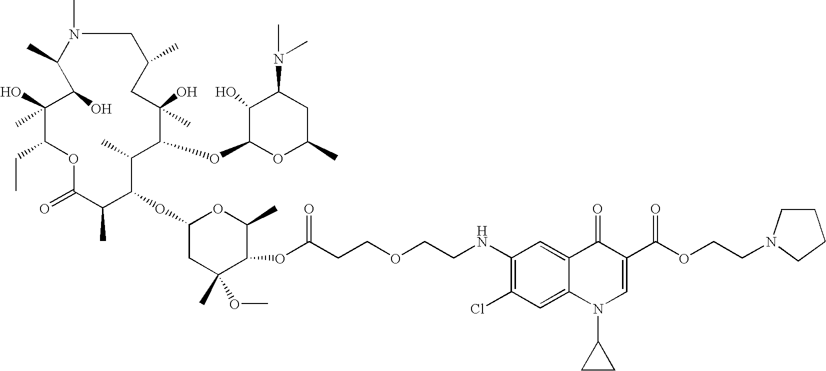

To a solution of Example 18 (0.4 g, 0.36 mmol) in DMF (10 mL), K₂CO₃ (0.64 g, 4.6 mmol) was added. After 90 minutes of stirring at room temperature 1-(2-chloroethyl)pyrrolidone hydrochloride (0.184 g, 1.08 mmol) and BTEAC (0.082 g, 0.36 mmol) were added and stirring was continued at 40° C. for 24 hours. After 4 hours, 5 eq. of K₂CO₃, 3 eq. of 1-(2-chloroethyl)pyrrolidone hydrochloride and 1 eq. of BTEAC were added. After 24 hours reaction still wasn't complete so another portion of 5 eq. of K₂CO₃, 3 eq. of 1-(2-chloroethyl)pyrrolidone hydrochloride and 1 eq. of BTEAC was added and after 24 hours one more portion and the reaction mixture was stirred for another 24 hours at 40° C. (a total of 72 hours). The reaction mixture was then diluted with water (50 mL) and extracted with EtOAc (3×20 mL). The combined organic layers were washed with brine (2×50 mL), dried and evaporated. The product was purified by column chromatography (sp (10 g), eluent: CH₂Cl₂:MeOH:NH₃=90:3:0.5) yielding 0.096 g of the title compound. MS; m/z (ES): 1222.6 [MH]⁺.

### Example 68

### 4″-O-(3-{2-[7-Chloro-1-cyclopropyl-3(2,2-dimethyl-propionyloxymethoxycarbonyl)-4-oxo-1,4-dihydro-quinolin-6-ylamino]-ethoxy}-propionyl)-azithromycin

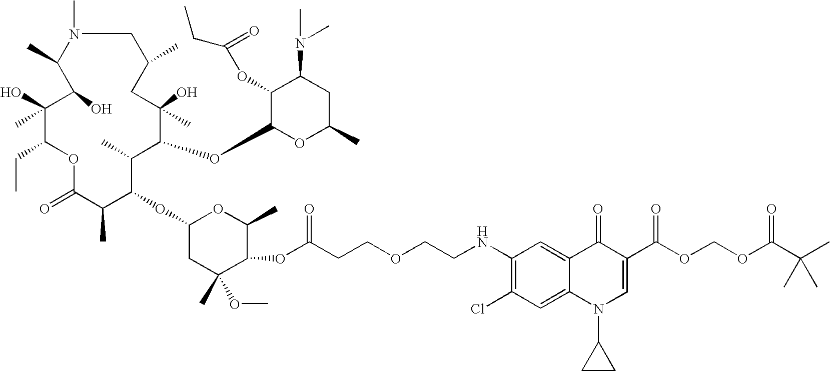

To a mixture of Example 45 (0.25 g, 0.21 mmol) in DMF (5 mL), K₂CO₃ (0.146 g, 1.06 mmol) was added. After 90 minutes, chloromethyl pivalate (0.045 g, 0.32 mmol) was added and the reaction mixture was stirred at 35° C. for 24 hours then diluted with water (50 mL) and extracted with EtOAc (2×30 mL). The organic layer was evaporated in vacuo. Precipitation from EtOAc:n-hexane yielded 0.197 g of crude product which was purified by column chromatography (sp (10 g), eluent: CH₂Cl₂/MeOH/NH₃=90/3/0.5). Another precipitation from EtOAc:n-hexane yielded 0.075 g of the title compound. MS; m/z (ES): 1295.6 [MH]⁺.

### Example 69

### 4″-O-(3-{2-[3-(4-Acetoxy butoxycarbonyl)-7-chloro-1-cyclopropyl-4-oxo-1,4-dihydro-quinolin-6-ylamino]-ethoxy}-propionyl)-azithromycin

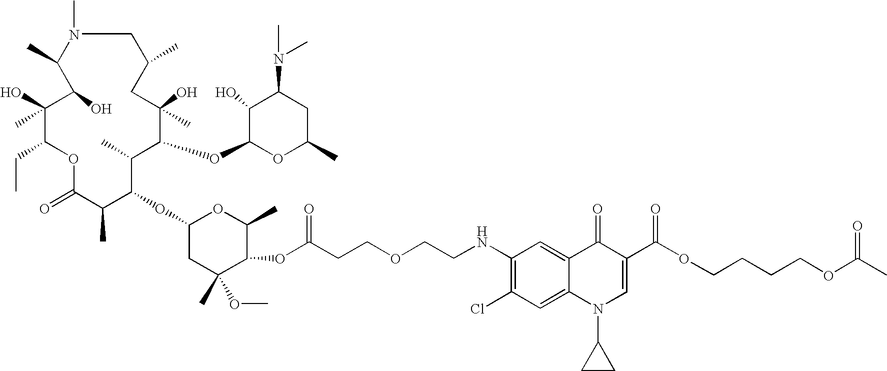

To a solution of Example 18 (0.4 g) in DMF (10 mL), K₂CO₃ (0.64 g) was added. After 2 hours, 4-bromobutylacetate (0.156 mL) was added and the reaction mixture was stirred at 50° C. for 24 hours, then diluted with water (60 mL) and extracted with EtOAc (3×25 mL). The combined organic layers were washed with 40 mL of brine, dried and evaporated. The product was purified by column chromatography (sp (10 g), eluent CH₂Cl₂/MeOH/NH₃:90:5:0.5). Precipitation from EtOAc:n-hexane yielded 0.176 g of the title compound. MS; m/z (ES): 1238.3 [MH]⁺.

### Example 70

### 4″-O-(3-{2-[7-Chloro-1-cyclopropyl-3-(1-ethoxycarbonyloxy-ethoxycarbonyl)-4-oxo-1,4-dihydro-quinolin-6-ylamino]-ethoxy}-propionyl)-2′-propionylazithromycin

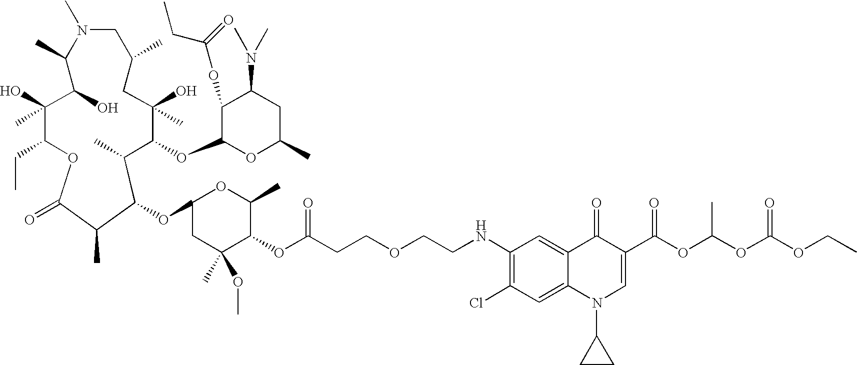

To a solution of Example 45 (0.360 g) in DMF (7 mL), were added 1-chloroethylethylcarbonate (0.140 mL), K₂CO₃ (0.211 g) and BTEAC (0.070 g). The mixture was stirred at 35° C. for 48 hours. The reaction mixture was then extracted with EtOAc (2×20 mL) and water (2×20 mL). The organic layer was extracted with NaHCO₃ (2×20 mL), dried over K₂CO₃ and evaporated in vacuo. The crude product (0.310 g) was purified by column chromatography (DCM:MeOH:NH₃=90:5:0.5) to yield crude product (0.142 g). This crude product (0.142 g) was purified by column chromatography (DCM:MeOH:NH₃=90:3:0.5) yielding the title compound (0.110 g). MS (ES+) m/z: [MH₂]²⁺=649.5.

### Example 71

### 4″-O-(3-{2-[1-Cyclopropyl-3-(1-ethoxycarbonyloxy-ethoxycarbonyl)-4-oxo-1,4-dihydro-quinolin-6-yloxy]-ethoxy}-propionyl)-azithromycin

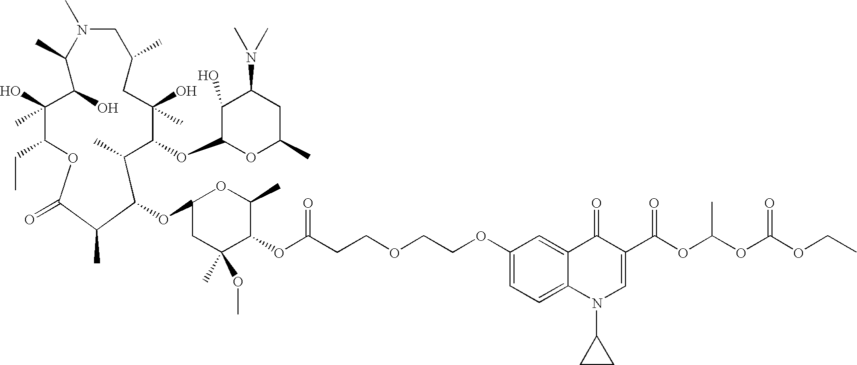

To a solution of Example 29 (0.297 g) in DMF (5 mL), was added K₂CO₃ (0.188 g). The mixture was stirred at room temperature for 1.5 hours, then chloroethylethyl carbonate (0.0475 mL) was added and the mixture was stirred at 35° C. for 24 hours. Another portion of chloroethylethyl carbonate (0.0475 mL) was then added and the reaction mixture was stirred at 35° C. for another 24 h. Water was added to the solution and the precipitate was filtered and dried in vacuo. The product was purified by column chromatography (SPE-column gradient polarity: 100% DCM to DCM:MeOH:NH₃=90:9:0.5) yielding 44 mg of the title compound. MS (ES+)m/z: [MH₂]²⁺=605.31.

### Example 72

### 4″-O-(3-{2-[1-Cyclopropyl-3-(2-methoxy-ethoxymethoxycarbonyl)-4-oxo-1,4-dihydro-quinolin-6-yloxy]-ethoxy}propionyl)-azithromycin

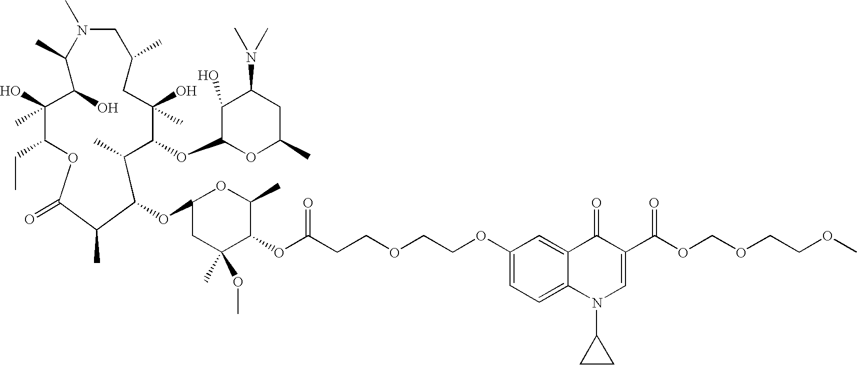

To a solution of Example 29 (0.3 g) in DMF (10 mL), were added MEM-chloride (0.0628 mL), K₂CO₃ (0.114 g) and BTEAC (0.063 g). The mixture was stirred at room temperature for 2 hours. The reaction mixture was then extracted with EtOAc (2×20 mL) and water (2×20 mL). The organic layer was dried over K₂CO₃ and evaporated in vacuo. The product was precipitated from EtOAc:diisopropyl-ether yielding 0.048 g of the title compound. MS (ES+)m/z: [MH]⁺=1180.47.

### Example 73

### 4″-O-(3-{2-[1-Cyclopropyl-4-oxo-3-(2-oxo-propoxycarbonyl)-1,4-dihydro-quinolin-6-yloxy]-ethoxy}-propionyl)-azithromycin

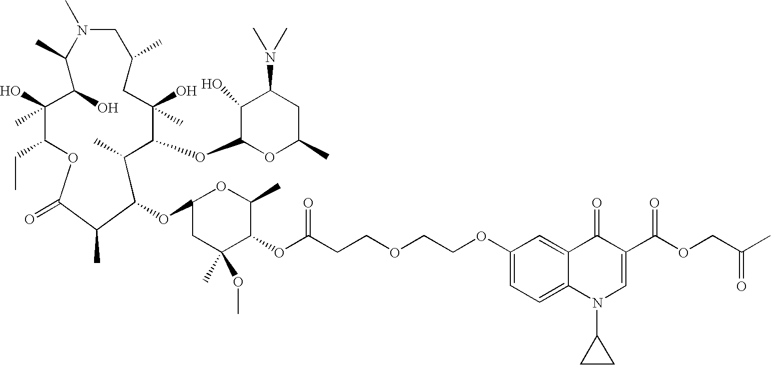

To a solution of Example 29 (0.188 g) in DMF (6.3 mL), were added 1-chloro-propan-2-one (0.02739 mL), K₂CO₃ (0.071 g) and BTEAC (0.039 g). The mixture was stirred at room temperature for 2 hours. The reaction mixture was then extracted with EtOAc (2×20 mL) and water (2×20 mL). The organic layer was dried over K₂CO₃ and evaporated in vacuo. The product was precipitated from EtOAc:diisopropylether yielding 0.115 g of the title compound. MS(ES+)m/z: [MH]⁺=1148.42.

### Example 74

### 4″-O-(3-{2-[1-Cyclopropyl-4-oxo-3-(2-piperidin-1-yl-ethoxycarbonyl)-1,4-dihydro-quinolin-6-ylamino]-ethoxy}-propionyl)-azithromycin

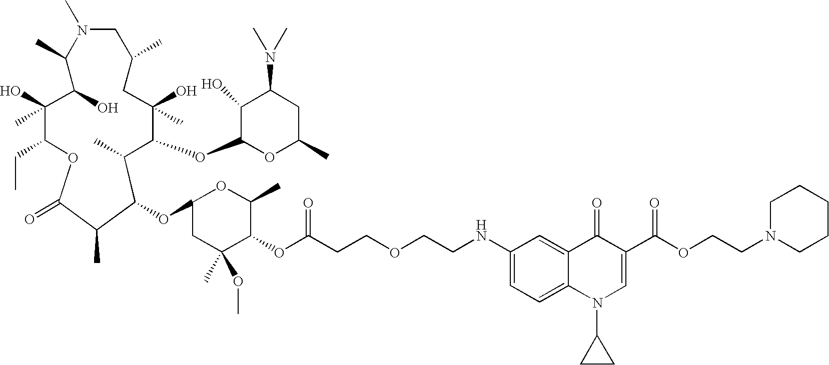

To a solution of Example 19 (0.2 g) in DMF (5 mL), was added K₂CO₃ (0.050 g). The mixture was stirred at room temperature for 1.5 hours and then 1-(2-chloroethyl)piperidine hydrochloride (0.067 g) was added. The reaction mixture was stirred at room temperature overnight. 1-(2-Chloroethyl)piperidine hydrochloride (0.067 g), BTEAC (0.042 g) and K₂CO₃ (0.253 g) were added and the reaction mixture was stirred at room temperature overnight. The reaction mixture was then extracted with EtOAc and water (2×10 mL). The organic layer washed with NaHCO₃ (2×20 mL) and NaCl (2×20 mL), dried over K₂CO₃ and evaporated in vacuo. The product was precipitated from EtOAc diisopropyl-ether yielding 0.090 g of the title compound. MS (ES+)m/z: [MH₂]²⁺=547.83.

### Example 75

### 4″-O-(3-{2-[1-Cyclopropyl-4-oxo-3-(2-oxo-propoxycarbonyl)-1,4-dihydro-quinolin-6-ylamino]-ethoxy}-propionyl)-azithromycin

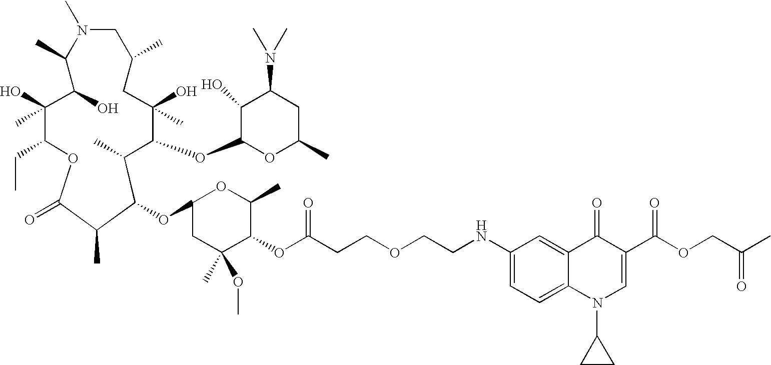

To a solution of Example 19 (0.30 g) in DMF (10 mL), were added 1-chloro-propan-2-one (0.04379 mL), K₂CO₃ (0.076 g) and BTEAC (0.063 g). The mixture was stirred at room temperature overnight. The reaction mixture was then extracted with EtOAc (2×20 mL) and water (2×20 mL). The organic layer was dried over K₂CO₃ and evaporated in vacuo. The product was precipitated from EtOAc:diisopropyl-ether yielding crude product (0.30 g). A portion of the crude product (0.20 g) was purified by column chromatography (DCM:MeOH:NH₃=90:3:0.5) yielding 0.120 g of the title compound. MS(ES+)m/z: [MH₂]²⁺=574.99.

### Example 76

### 4″-O-(3-{2-[1-Cyclopropyl-3-(2-methoxy-ethoxymethoxycarbonyl)-4-oxo-1,4-dihydro-quinolin-6-ylamino]-ethoxy}-propionyl)-2′-O-propionylazithromycin

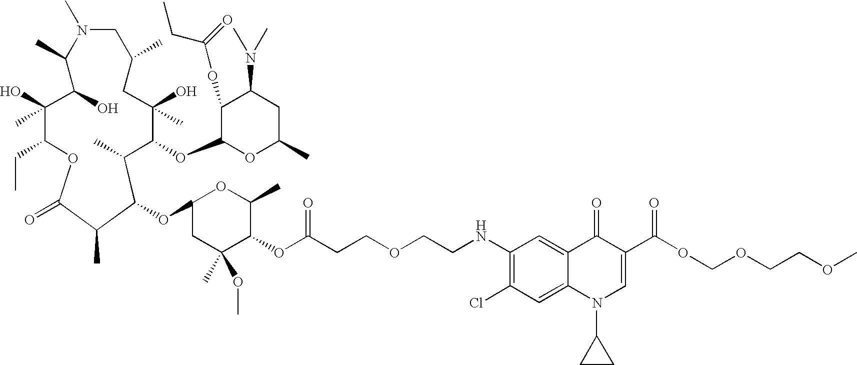

To a solution of Example 45 (0.150 g) in DMF (5 mL), were added MEM-chloride (0.029 mL), K₂CO₃ (0.053 g) and BTEAC (0.029 g). The mixture was stirred at room temperature for 4 hours. The reaction mixture was then extracted with EtOAc (2×20 mL) and water (2×20 mL). The organic layer was extracted with NaHCO₃ (2×20 mL), dried over K₂CO₃ and evaporated in vacuo. The product (0.123 g) was precipitated from EtOAc:diisopropyl-ether yielding crude title compound (0.066 g). MS (ES+) m/z: [MH₂]²⁺=634.05.

### Example 77

### 4″-O-{3-[2-(7-Chloro-1-cyclopropyl-3-isopropoxycarbonyl-4-oxo-1,4-dihydro-quinolin-6-ylamino)-ethoxy]-propionyl}-2′-O-propionylazithromycin

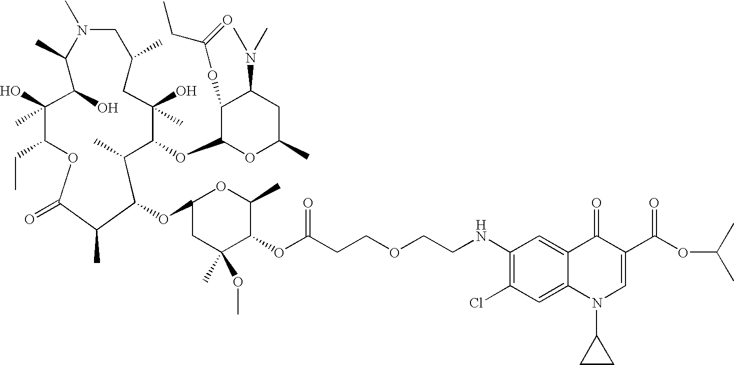

To a solution of Example 45 (0.4 g) in DMF (12.5 mL), were added 2-iodo-propane (0.10122 mL), K₂CO₃ (1.216 g) and BTEAC (0.1544 g). The mixture was stirred at room temperature for 24 hours. The reaction mixture was then extracted with EtOAc (2×20 mL) and water (2×20 mL). The organic layer was extracted with NaHCO₃ (2×20 mL), dried over K₂CO₃ and evaporated in vacuo. The crude product (0.401 g) was purified by column chromatography (DCM:MeOH:NH₃=90:3:0.5) yielding the title compound (0.132 g). MS (ES+) m/z: [MH₂]²⁺=612.4.

### Example 78

### 4″-O-(3-{2-[7-Chloro-1-cyclopropyl-4-oxo-3-(2-piperidin-1-yl-ethoxycarbonyl)-1,4-dihydro-quinolin-6-ylamino]-ethoxy}-propionyl)-azithromycin

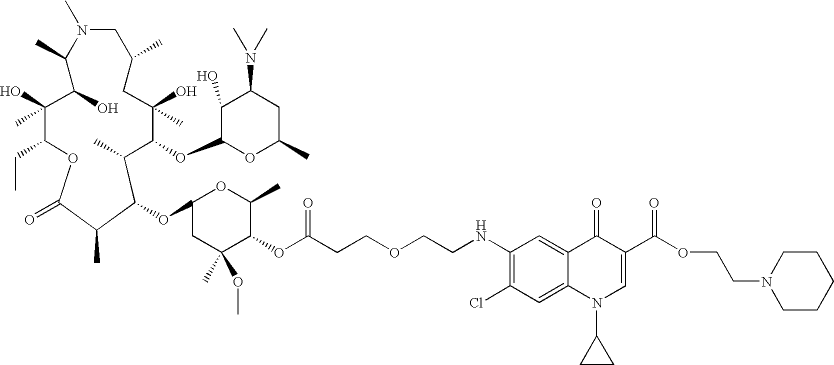

To a solution of Example 18 (0.4 g) in DMF (10 mL), was added K₂CO₃ (0.590 g). The mixture was stirred at room temperature for 1.5 hours and then 1-(2-chloroethyl)piperidine hydrochloride (0.262 g) was added. The reaction mixture was stirred at room temperature overnight. The reaction mixture was then extracted with EtOAc and water (2×20 mL). The organic layer washed with NaHCO₃ (2×20 mL) and NaCl (2×20 mL), dried over K₂CO₃ and evaporated in vacuo. The product was precipitated from EtOAc:diisopropyl-ether yielding the title compound (0.352 g). MS (ES+) m/z: [MH₂]²⁺=619.0.

### Example 79

### 4″-O-{3-[2-(7-Chloro-3-cyclobutylmethoxycarbonyl-1 cyclopropyl-4-oxo-1,4-dihydro-quinolin-6-ylamino)ethoxy]-propionyl}-azithromycin

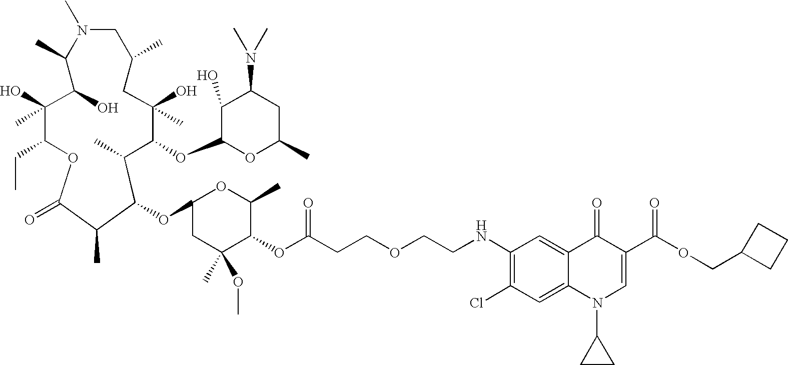

To a solution of Example 18 (0.4 g) in DMF (10 mL), was added K₂CO₃ (0.638 g). The mixture was stirred at 50° C. for 24 hours, then was heated to 80° C. for 6 hours, and then at 50° C. for 48 hours. The reaction mixture then extracted with EtOAc and water (2×20 mL). The organic layer washed with NaCl (2×20 mL), dried over K₂CO₃ and evaporated in vacuo. The crude product (0.273 g) was purified by column chromatography (DCM:MeOH:NH₃=90:5:0.5) yielding the title compound (0.07 g). MS (ES+) m/z: [MH₂]⁺=1194.8.

### General Procedure for the Preparation of Examples 80 to 87

Examples 80 to 87 were prepared by parallel synthesis using the following procedure:

PS-TBD resin (4.5 equiv., 88 mg, 1.31 mmol/g) was incubated with a solution of Example 18 (1.1 eq) in 1.7 mL THF for 1 h. The halide (15.0 eq) was added to the reaction vessel and the reaction was carried out at 60° C. for 24 h. The filtrate was purified on a SPE column (SiO₂, DCM/MeOH—NH₃(10:1); 100-84.5) to yield the desired product.

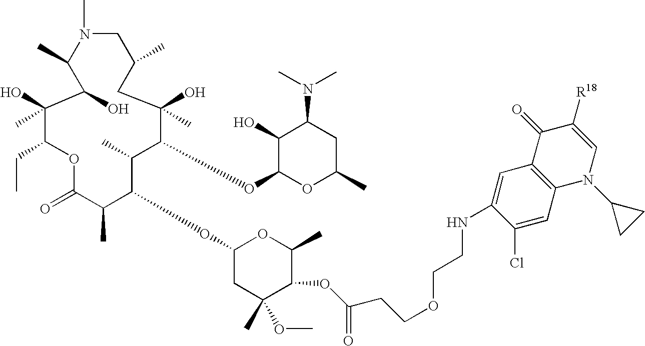

| Example No. | Compound Name | R¹⁸ | Yield | MS MH⁺) | Purity |
|---|---|---|---|---|---|
| Example 80 | 4″-O-{3-[2-(7- Chloro-3-(3- cyanopropoxy carbonyl)-1- cyclopropyl-4-oxo- 1,4-dihydro- quinolin-6- ylamino)-ethoxy]- propionyl}- azithromycin | 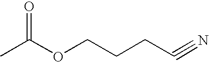 | 15.6 mg | 1193.2 | 94.6% |
| Example 81 | 4″-O-{3-[2-(7- Chloro-3- allyloxycarbonyl-1- cyclopropyl-4-oxo 1,4-dihydro- quinolin-6- ylamino)-ethoxy]- propionyl}- azithromycin | 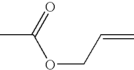 | 7.3 mg | 1166.2 | 8% |
| Example 82 | 4″-O-{3-[2-(7- Chloro-3-(4- nitrophenylmethoxy carbonyl)-1- cyclopropyl-4-oxo- 1,4-dihydro- quinolin-6- ylamino)-ethoxy]- propionyl}- azithromycin | 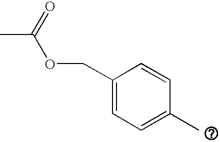 | 8.2mg | 1261.2 | 80% |
| Example 83 | 4″-O-{3-[2-(7- Chloro-3-(4- acetoxymethylphenyl methoxycarbonyl)-1- cyclopropyl-4-oxo- 1,4-dihydro- quinolin-6- ylamino)-ethoxy]- propionyl}- azithromycin | 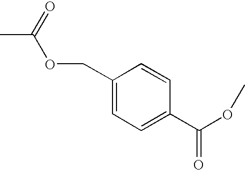 | 15.2 mg | 1275.3 | 80% |
| Example 84 | 4″-O-{3-[2-(7- Chloro-3- phenylethoxycarbonyl-1- cyclopropyl-4-oxo- 1,4-dihydro- quinolin-6- ylamino)-ethoxy]- propionyl}- azithromycin | 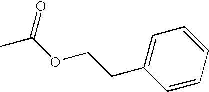 | 7.1 mg | 1230.4 | 80% |
| Example 85 | 4″-O-{3-[2-(7- Chloro-3- phenylmethoxycabonyl- 1-cyclopropyl-4-oxo- 1,4-dihydro- quinolin-6- ylamino)-ethoxy]- propionyl}- azithromycin | 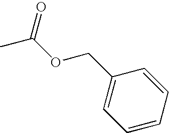 | 10.3 mg | 1216.3 | 85% |
| Example 86 | 4″-O-{3-[2-(7- Chloro-3-(4- acetoxy- butoxycarbonyl)-1- cyclopropyl-4-oxo- 1,4-dihydro- quinolin-6- ylamino)-ethoxy]- propionyl}- azithromycin | 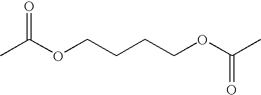 | 12.9 mg | 1239.6 | 82% |
| Example 87 | 4″-O-{3-[2-(7- Chloro-3-(4- acetoxy- pentoxycarbonyl)- 1-cyclopropyl-4 -oxo-1,4-dihydro- quinolin-6- ylamino)-ethoxy]- propionyl}- azithromycin | 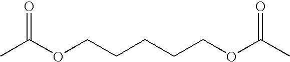 | 12.8 mg | 1253.6 | 90% |

Biological Data

Using a standard broth dilution method in microtitre, compounds were tested for antibacterial activity. The compounds in the above examples gave minimum inhibitory concentrations (MICs) less than 1 microgram per millilitre against erythromycin-sensitive and erythromycin-resistant strains of Streptococcus pneumoniae and Streptococcus pyogenes.

In addition, the MIC (μg/mL) of test compounds against various organisms was determined including:

S. aureus Smith ATCC 13709, S. pneumoniae SP030, S. pyogenes 3565, E. faecalis ATCC 29212, H. influenzae ATCC 49247, M. catarrhalis ATCC 23246.

Examples 1, 2, 4-7, 9-14, 17-19, 22, 23, 25-29, 31-37 and 40 have an MIC≦1 μg/mL against S. aureus Smith ATCC 13709, S. pneumoniae SP030, S. pyogenes 3565 and E. faecalis ATCC 29212.

Examples 1-4, 6, 9, 11, 13, 17-19, 22, 23, 26, 27, 29, 31, 33-35 and 40 have an MIC≦2 μg/mL against H. influenzae ATCC 49247 and M. catarrhalis ATCC 23246.

Examples 5-7, 9, 11, 13, 15, 17-19, 23, 27-29 and 32 have an MIC≦0.25 μg/mL against erythromycin resistant strains of Streptococcus pneumoniae and Streptococcus pyogenes.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 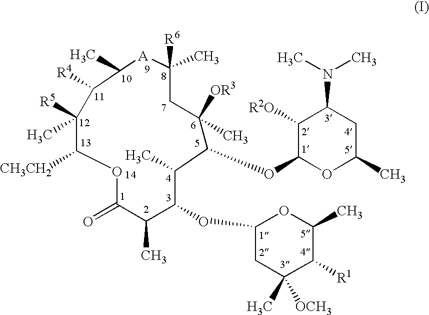
wherein
A is a bivalent radical selected from —C(O)—, —C(O)NH—, —NHC(O)—, —N(R⁷)—CH₂—, —CH₂—N(R⁷)—, —CH(NR⁸R⁹)— and —C(═NR¹⁰)—;
R¹ is —OC(O)(CH₂)_{d}XR¹¹;
R² is hydrogen or a hydroxyl protecting group;
R³ is hydrogen, C₁₋₄alkyl, or C₃₋₆alkenyl optionally substituted by 9 to 10 membered fused bicyclic heteroaryl;
R⁴ is hydroxy, C₃₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R¹²,
R⁵ is hydroxy, or
R⁴ and R⁵ taken together with the intervening atoms form a cyclic group having the following structure: 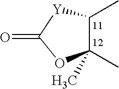
wherein Y is a bivalent radical selected from —CH₂—, —CH(CN)—, —O—, —N(R¹³)— and —CH(SR¹³)—;
R⁶ is hydrogen or fluorine;
R⁷ is hydrogen or C₁₋₆alkyl;
R⁸ and R⁹ are each independently hydrogen, C₁₋₆alkyl, —C(═NR¹⁰)NR¹⁴R¹⁵ or —C(O)R¹⁴, or
R⁸ and R⁹ together form ═CH(CR¹⁴R¹⁵)_{f}aryl, ═CH(CR¹⁴R¹⁵)_{f}heterocyclyl, ═CR¹⁴R¹⁵ or ═C(R¹⁴)C(O)OR¹⁴, wherein the alkyl, aryl and heterocyclyl groups are optionally substituted by up to three groups independently selected from R¹⁶;
R¹⁰ is —OR¹⁷, C₁₋₆alkyl, —(CH₂)_{g}aryl, —(CH₂)_{g}heterocyclyl or —(CH₂)ₕ—O(CH₂)ᵢOR⁷, wherein each R¹⁰ group is optionally substituted by up to three groups independently selected from R¹⁶;
R¹¹ is a heterocyclic group having the following structure: 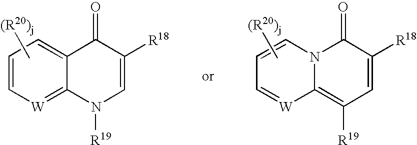
R¹² is hydrogen or C₁₋₆alkyl;
R¹³ is hydrogen or C₁₋₄alkyl optionally substituted by a group selected from optionally substituted phenyl, optionally substituted 5 or 6 membered heteroaryl and optionally substituted 9 to 10 membered fused bicyclic heteroaryl;
R¹⁴ and R¹⁵ are each independently hydrogen or C₁₋₆alkyl;
R¹⁶ is halogen, cyano, nitro, trifluoromethyl, azido, —C(O)R²¹, —C(O)OR²¹, —OC(O)R²¹, —OC(O)OR²¹, —NR²²C(O)R²³, —C(O)NR²²R²³, —NR²²R²³, hydroxy, C₁₋₆alkyl, —S(O)ₖC₁₋₆alkyl, C₁₋₆alkoxy, —(CH₂)ₘaryl or —(CH₂)ₘheteroaryl, wherein the alkoxy group is optionally substituted by up to three groups independently selected from —NR¹⁴R¹⁵, halogen and —OR¹⁴, and the aryl and heteroaryl groups are optionally substituted by up to five groups independently selected from halogen, cyano, nitro, trifluoromethyl, azido, —C(O)R²⁴, —C(O)OR²⁴, —OC(O)OR²⁴, —NR²⁵C(O)R²⁶, —C(O)NR²⁵R²⁶, —NR²⁵R²⁶, hydroxy, C₁₋₆alkyl and C₁₋₆alkoxy;
R¹⁷ is hydrogen, C₁₋₆alkyl, C₃₋₇cycloalkyl, C₃₋₆alkenyl or a 5 or 6 membered heterocyclic group, wherein the alkyl, cycloalkyl, alkenyl and heterocyclic groups are optionally substituted by up to three substituents independently selected from optionally substituted 5 or 6 membered heterocyclic group, optionally substituted 5 or 6 membered heteroaryl, —OR²⁷, —S(O)ₙR²⁷, —NR²⁷R²⁸, —CONR²⁷R²⁸, halogen and cyano;
R¹⁸ is hydrogen, —C(O)OR²⁹, —C(O)NHR²⁹, —C(O)CH₂NO₂ or —C(O)CH₂SO₂R⁷;
R¹⁹ is hydrogen, C₁₋₄alkyl optionally substituted by hydroxy or C₁₋₄alkoxy, C₃₋₇cycloalkyl, or optionally substituted phenyl or benzyl;
R²⁰ is halogen, C₁₋₄alkyl, C₁₋₄thioalkyl, C₁₋₄alkoxy, —NH₂, —NH(C₁₋₄alkyl) or —N(C₁₋₄alkyl)₂;
R²¹ is hydrogen, C₁₋₁₀alkyl, —(CH₂)ₚaryl or —(CH₂)ₚheteroaryl;
R²² and R²³ are each independently hydrogen, —OR¹⁴, C₁₋₆alkyl, —(CH₂)_{q}aryl or —(CH₂)_{q}heterocyclyl;
R²⁴ is hydrogen, C₁₋₁₀alkyl, —(CH₂)ᵣaryl or —(CH₂)ᵣheteroaryl;
R²⁵ and R²⁶ are each independently hydrogen, —OR¹⁴, C₁₋₆alkyl, —(CH₂)ₛaryl or —(CH₂)ₛheterocyclyl;
R²⁷ and R²⁸ are each independently hydrogen, C₁₋₄alkyl or C₁₋₄alkoxyC₁₋₄alkyl;
R²⁹ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR³²R³³, —NR³²R³³ and phenyl optionally substituted by nitro or —C(O)OC₁₋₆alkyl,
—(CH₂)_{w}C₃₋₇cycloalkyl,
—(CH₂)_{w}heterocyclyl,
—(CH₂)_{w}heteroaryl,
—(CH₂)_{w}aryl,
C₃₋₆alkenyl, or
C₃₋₆alkynyl;
R³⁰ is hydrogen, C₁₋₄alkyl, C₃₋₇cycloalkyl, optionally substituted phenyl or benzyl, acetyl or benzoyl;
R³¹ is hydrogen or R²⁰, or R³¹ and R¹⁹ are linked to form the bivalent radical —O(CH₂)₂— or —(CH₂)ₜ—;
R³² and R³³ are each independently hydrogen or C₁₋₆alkyl optionally substituted by phenyl or —C(O)OC₁₋₆alkyl, or
R³² and R³³, together with the nitrogen atom to which they are bound, form a 5 or 6 membered heterocyclic group optionally containing one additional heteroatom selected from oxygen, nitrogen and sulfur;
X is —U(CH₂)ᵥB—;
U is —N(R³⁰)— and B is —O— or —S(O)_{z}, or
U is —O— and B is —N(R³⁰)— or —O—;
W is —C(R³¹)— or a nitrogen atom;
d is 0 or an integer from 1 to 5;
e is an integer from 2 to 4;
f, g, h, m, p, q, r and s are each independently integers from 0 to 4;
i is an integer from 1 to 6;
j, k, n and z are each independently integers from 0 to 2;
t is 2 or 3;
v is an integer from 1 to 8;
or a pharmaceutically acceptable derivative thereof.

2. A compound according to claim 1 wherein A is —C(O)— or —N(R⁷)—CH₂—.

3. A compound according to claim 1 wherein d is 2.

4. A compound according to claim 1 wherein v is 2.

5. A compound according to claim 1 wherein R¹¹ is a heterocyclic group of the following formula: 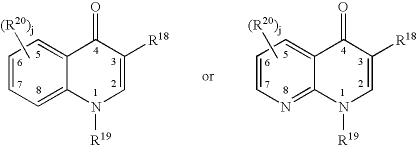
wherein the heterocyclic is linked in the 6 or 7 position and j, R¹⁸, R¹⁹ and R²⁰ are as defined in claim 1, or a heterocyclic group of the following formula: 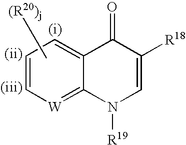
wherein the heterocylic is linked in the (ii) or (iii) position, W is —C(R³¹)— and R³¹ and R¹⁹ are linked to form the bivalent radical —(CH₂)ₜ— as defined in claim 1, and j, R¹⁸, R¹⁹ and R²⁰ are as defined in claim 1.

6. A compound according to claim 1 as defined in any one of Examples 1 to 87, or a pharmaceutically acceptable derivative thereof.

7. A compound selected from:
4″-O-{13-[2-(3-carboxy-1-ethyl-4-oxo-1,4-dihydro-6-quinolinylsulfanyl)ethylamino]propionyl}-6-O-methyl-erythromycin A;
4″-O-{3-[2-(3-carboxy-1-ethyl-4-oxo-1,4-dihydro-6-quinolinylsulfanyl)ethylamino]propionyl}-6-O-methyl-11-desoxy-11-(R)-amino-erythromycin A11,12-carbamate;
4″-O-{3-[2-(3-carboxy-1-ethyl-4-oxo-1,4-dihydro-6-quinolinysulfanyl)ethylamino]propionyl}-azithromycin11,12-carbonate;
4″-O-{3-[2-(6-carboxy-7-oxo-2,3-dihydro-1H,7H-pyrido[3,2,1-ij]quinolin-9-yloxy)ethylamino]propionyl}-6-O-methyl-erythromycin A;
4″-O-{3-[2-(3-carboxy-1-ethyl-4-oxo-1,4-dihydro-7-quinolinyloxy)ethylamino]propionyl}-6-O-methyl-erythromycin A;
4″-O-{3-[2-(3-carboxy-7-chloro-1-cyclopropyl-4-oxo-1,4-dihydro-6-quinolinylamino)ethoxy]propionyl}-azithromycin;
4″-O-{3-[2-(3-carboxy-1-cyclopropyl-4-oxo-1,4-dihydro-6-quinolinylamino)ethoxy]propionyl}-azithromycin;
4″-O-{3-[2-(3-carboxy-7-chloro-1-cyclopropyl-4-oxo-1,4-dihydro-6-quinolinylamino)ethoxy]propionyl}-11-O-methyl-azithromycin;
4″-O-{3-[2-(3-carboxy-7-chloro-1-cyclopropyl-4-oxo-1,4-dihydro-quinolin-6-yloxy)-ethoxy]-propionyl}-azithromycin; and
4″-O-{3-[2-(3-carboxy-1-cyclopropyl-4-oxo-1,4-dihydro-quinolin-6-yloxy)-ethoxy]-propionyl}-azithromycin;
4″-O-{3-[2-(3-carboxy-7-chloro-1-cyclopropyl-4-oxo-1,4-dihydro-6-quinolinylamino)ethoxy]propionyl}-azithromycin11,12-cyclic carbonate;
4″-O-{3-[2-(3-carboxy-1-cyclopropyl-4-oxo-1,4-dihydro-6-quinolinylamino)ethoxy]propionyl}-11-O-methyl-azithromycin;
4″-O-{3-[2-(3-carboxy-7-chloro-1-cyclopropyl-4-oxo-1,4-dihydro-quinolin-6-yloxy)-ethoxy]-propionyl}-azithromycin11,12-carbonate;
4″-O-{3-[2-(3-carboxy-7-chloro-1-cyclopropyl-4-oxo-1,4-dihydro-6-quinolinylamino)ethoxy]propionyl}-6-O-methyl-1-desoxy-11-(R)-amino-erythromycin A11,12-carbamate;
4″-O-{3-[2-(3-carboxy-1-cyclopropyl-4-oxo-1,4-dihydro-quinolin-6-yloxy)-ethoxy]-propionyl}-11-O-methyl-azithromycin;
4″-O-{3-[2-(3-carboxy-7-chloro-1-cyclopropyl-4-oxo-1,4-dihydro-quinolin-6-yloxy)ethoxy]propionyl}-6-O-methyl-erythromycin A;
4″-O-{3-[2-(3-carboxy-1-cyclopropyl-6-fluoro-8-methoxy-4-oxo-1,4-dihydro-quinolin-7-ylamino)ethoxy]propionyl}-azithromycin;
or a pharmaceutically acceptable derivative thereof.

8. A process for the preparation of a compound as claimed in claim 1 which comprises:
a) reacting a compound of formula (II) 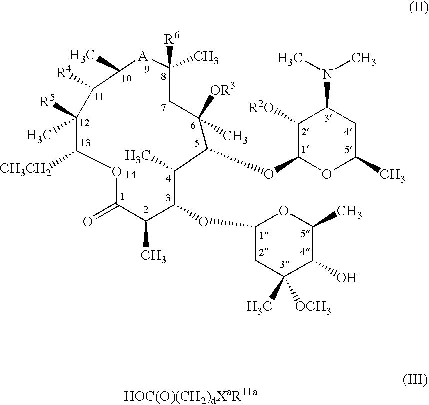
with a suitable activated derivative of the acid (III), wherein X^{a} and R^{11a} are X and R¹¹ as defined in claim 1 or groups convertible to X and R¹¹, to produce a compound of formula (I) wherein d is an integer from 1 to 5;
b) reacting a compound of formula (II), in which the 4″ hydroxy is suitably activated, with a compound of formula X^{a}R^{11a} (IV), wherein R^{11a} is R¹¹ as defined in claim 1 or a group convertible to R¹¹ and X^{a} is —U(CH₂)ᵥB— or a group convertible to —U(CH₂)ᵥB—, in which U is a group selected from selected from —N(R³⁰)— and —O—, to produce a compound of formula (I) wherein d is 0 and U is a group selected from —N(R³⁰)— and —O—;
c) reacting a compound of formula (V) 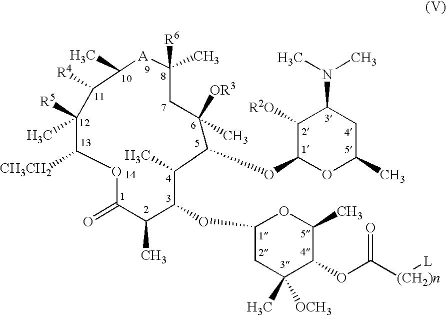
with a compound of formula X^{a}R^{11a} (IV), wherein R^{11a} is R¹¹ as defined in claim 1 or a group convertible to R¹¹ and X^{a} is —U(CH₂)ᵥB— or a group convertible to —U(CH₂)ᵥB— in which U is —N(R³⁰)—, and L is suitable leaving group, to produce a compound of formula (I) wherein U is —N(R³⁰)—;
d) reacting a compound of formula (VII), with a compound of formula X^{a}R^{11a} (IV), 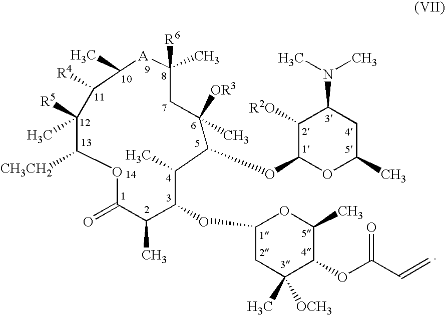
wherein R^{11a} is R¹¹ as defined in claim 1 or a group convertible to R¹¹, and X^{a} is —U(CH₂)ᵥB— or a group convertible to —U(CH₂)ᵥB— in which U is N(R³⁰)—, to produce a compound of formula (I) wherein d is 2 and U is —N(R³⁰)—; or
e) converting one compound of formula (I) into another compound of formula (I);
and thereafter, if required, subjecting the resulting compound to one or more of the following operations:
i) removal of the protecting group R²,
ii) conversion of X^{a}R^{11a} to XR¹¹,
iii) conversion of B^{a}R^{11a} to BR¹¹, and
iv) conversion of the resultant compound of formula (I) into a pharmaceutically acceptable derivative thereof.

9. A compound as claimed in claim 1 for use in therapy.

10-11. (canceled)

12. A method for the treatment of the human or non-human animal body to combat microbial infection comprising administration to a body in need of such treatment of an effective amount of a compound as claimed in claim 1.

13. A pharmaceutical composition comprising at least one compound as claimed in claim 1 in association with a pharmaceutically acceptable excipient, diluent and/or carrier.

14. A compound of formula (IA) 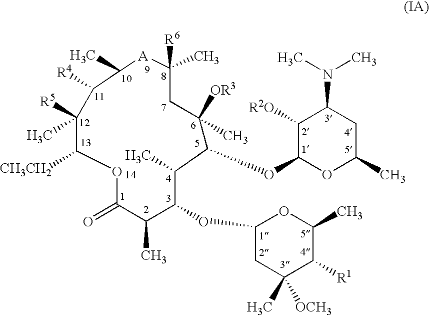
wherein
A is a bivalent radical selected from —C(O)—, —C(O)NH—, —NHC(O)—, —N(R⁷)—CH₂—, —CH₂—N(R⁷)—, —CH(NR⁸R⁹)— and —C(═NR¹⁰)—;
R¹ is —OC(O)(CH₂)_{d}XR¹¹;
R² is hydrogen or a hydroxyl protecting group;
R³ is hydrogen, C₁₋₄alkyl, or C₃₋₆alkenyl optionally substituted by 9 to 10 membered fused bicyclic heteroaryl;
R⁴ is hydroxy, C₃₋₆alkenyloxy optionally substituted by 9 to 10 membered fused bicyclic heteroaryl, or C₁₋₆alkoxy optionally substituted by C₁₋₆alkoxy or —O(CH₂)ₑNR⁷R¹²,
R⁵ is hydroxy, or
R⁴ and R⁵ taken together with the intervening atoms form a cyclic group having the following structure: 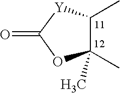
wherein Y is a bivalent radical selected from —CH₂—, —CH(CN)—, —O—, —N(R¹³)— and —CH(SR¹³)—;
R⁶ is hydrogen or fluorine;
R⁷ is hydrogen or C₁₋₆alkyl;
R⁸ and R⁹ are each independently hydrogen, C₁₋₆alkyl, —C(═NR¹⁰)NR¹⁴R¹⁵ or —C(O)R¹⁴, or
R⁸ and R⁹ together form ═CH(CR¹⁴R¹⁵)_{f}aryl, ═CH(CR¹⁴R¹⁵)_{f}heterocyclyl, ═CR¹⁴R¹⁵ or ═C(R¹⁴)C(O)OR¹⁴, wherein the alkyl, aryl and heterocyclyl groups are optionally substituted by up to three groups independently selected from R¹⁶;
R¹⁰ is —OR¹⁷, C₁₋₆alkyl, —(CH₂)_{g}aryl, —(CH₂)_{g}heterocyclyl or —(CH₂)ₕO(CH₂)ᵢOR⁷, wherein each R¹⁰ group is optionally substituted by up to three groups independently selected from R¹⁶;
R¹¹ is a heterocyclic group having the following structure: 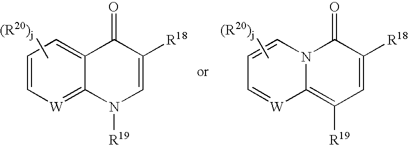
R¹² is hydrogen or C₁₋₆alkyl;
R¹³ is hydrogen or C₁₋₄alkyl substituted by a group selected from optionally substituted phenyl, optionally substituted 5 or 6 membered heteroaryl and optionally substituted 9 to 10 membered fused bicyclic heteroaryl;
R¹⁴ and R¹⁵ are each independently hydrogen or C₁₋₆alkyl;
R¹⁶ is halogen, cyano, nitro, trifluoromethyl, azido, —C(O)R²¹, —C(O)OR²¹, —OC(O)R²¹, —OC(O)OR²¹, —NR²²C(O)R²³, —C(O)NR²²R²³, —NR²²R²³, hydroxy, C₁₋₆alkyl, —S(O)ₖC₁₋₆alkyl, C₁₋₆alkoxy, —(CH₂)ₘaryl or —(CH₂)ₘheteroaryl, wherein the alkoxy group is optionally substituted by up to three groups independently selected from —NR¹⁴R¹⁵, halogen and —OR¹⁴, and the aryl and heteroaryl groups are optionally substituted by up to five groups independently selected from halogen, cyano, nitro, trifluoromethyl, azido, —C(O)R²⁴, —C(O)OR²⁴, —OC(O)OR²⁴, —NR²⁵C(O)R²⁶, —C(O)NR²⁵R²⁶, —NR²⁵R²⁶, hydroxy, C₁₋₆alkyl and C₁₋₆alkoxy;
R¹⁷ is hydrogen, C₁₋₆alkyl, C₃₋₇cycloalkyl, C₃₋₆alkenyl or a 5 or 6 membered heterocyclic group, wherein the alkyl, cycloalkyl, alkenyl and heterocyclic groups are optionally substituted by up to three substituents independently selected from optionally substituted 5 or 6 membered heterocyclic group, optionally substituted 5 or 6 membered heteroaryl, —OR²⁷, —S(O)ₙR²⁷, —NR²⁷R²⁸, —CONR²⁷R²⁸, halogen and cyano;
R¹⁸ is hydrogen, —C(O)OR²⁹, —C(O)NHR²⁹ or —C(O)CH₂NO₂;
R¹⁹ is hydrogen, C₁₋₄alkyl optionally substituted by hydroxy or C₁₋₄alkoxy, C₃₋₇cycloalkyl, or optionally substituted phenyl or benzyl;
R²⁰ is halogen, C₁₋₄alkyl, C₁₋₄thioalkyl, C₁₋₄alkoxy, —NH₂, —NH(C₁₋₄alkyl) or —N(C₁₋₄alkyl)₂;
R²¹ is hydrogen, C₁₋₁₀alkyl, —(CH₂)ₚaryl or —(CH₂)ₚheteroaryl;
R²² and R²³ are each independently hydrogen, —OR¹⁴, C₁₋₆alkyl, —(CH₂)_{q}aryl or —(CH₂)_{q}heterocyclyl;
R²⁴ is hydrogen, C₁₋₁₀alkyl, —(CH₂)ᵣaryl or —(CH₂)ᵣheteroaryl;
R²⁵ and R²⁶ are each independently hydrogen, —OR¹⁴, C₁₋₆alkyl, —(CH₂)ₛaryl or —(CH₂)ₛheterocyclyl;
R²⁷ and R²⁸ are each independently hydrogen, C₁₋₄alkyl or C₁₋₄alkoxyC₁₋₄alkyl;
R²⁹ is hydrogen or C₁₋₆alkyl optionally substituted by up to three groups independently selected from halogen, C₁₋₄alkoxy, —OC(O)C₁₋₆alkyl and —OC(O)OC₁₋₆alkyl;
R³⁰ is hydrogen, C₁₋₄alkyl, C₃₋₇cycloalkyl, optionally substituted phenyl or benzyl, acetyl or benzoyl;
R³¹ is hydrogen or R²⁰, or R³¹ and R¹⁹ are linked to form the bivalent radical —O(CH₂)₂— or —(CH₂)ₜ—;
X is —U(CH₂)ᵥB—;
U is —N(R³⁰)— and B is —O— or —S(O)_{z}, or
U is —O— and B is —N(R³⁰)— or —O—;
W is —C(R³¹)— or a nitrogen atom;
d is 0 or an integer from 1 to 5;
e is an integer from 2 to 4;
f, g, h, m, p, q, r and s are each independently integers from 0 to 4;
i is an integer from 1 to 6;
j, k, n and z are each independently integers from 0 to 2;
t is 2 or 3;
v is an integer from 2 to 8;
or a pharmaceutically acceptable derivative thereof.
